(19) United States
(12) Patent Application Publication
(10) Pub. No.: US 2024/0329202 A1
(43) Pub. Date: Oct. 3, 2024

(54) SYSTEMS AND METHODS FOR CONTROLLING LASER TREATMENTS USING REFLECTED INTENSITY SIGNALS

(71) Applicant: IPG PHOTONICS CORPORATION, Marlborough, MA (US)

(72) Inventors: Gregory Altshuler, Lincoln, MA (US); Dmitriy Nikitin, Fryazino (RU); Daniil Myasnikov, Fryazino (RU); Anastasiya Kovalenko, Fryazino (RU); Viacheslav Brunov, Fryazino (RU); Dmitriy Protasenya, Fryazino (RU); Andrey Mashkin, Burbach (DE); ILYA Yaroslavsky, N. Andover, MA (US); Dmitri Boutoussov, Southborough, MA (US); Dilip Paithankar, Wayland, MA (US)

(73) Assignee: IPG PHOTONICS CORPORATION, Marlborough, MA (US)

(21) Appl. No.: 18/400,153

(22) Filed: Dec. 29, 2023

Related U.S. Application Data
(63) Continuation-in-part of application No. 18/019,356, filed on Feb. 2, 2023.
(60) Provisional application No. 63/062,118, filed on Aug. 6, 2020.
(60) Provisional application No. 63/184,970, filed on May 6, 2021.

(51) Int. Cl. G01S 7/48; G01S 7/481; G01S 7/4861; G01S 7/497; G01S 7/51; G01S 17/88

(52) U.S. Cl. CPC G01S 7/4802; G01S 7/4818; G01S 7/4861; G01S 7/497; G01S 7/51; G01S 17/88

(57) ABSTRACT

A method for controlling a surgical laser system that includes providing a surgical fiber configured to receive light reflected from a target in a surgical treatment area, and providing a computing device configured to couple with at least two photodetectors, each photodetector configured to detect an intensity of reflected light from the target in a different selected wavelength band, the computing device further configured to: receive the reflected light intensity in at least two selected wavelength bands, generate optical data corresponding to the reflected light intensity, and identify the target as a treatment target or a non-treatment target based at least in part on the optical data and a predetermined calibration based on at least two known targets.

### CROSS-REFERENCE TO RELATED APPLICATIONS

This application is a continuation in part of U.S. patent application Ser. No. 18/019,356, filed Aug. 6, 2021, which claims the benefit of U.S. Provisional Application No. 63/062,118, titled “Artificial Intelligent Assisted Laser Urology Platform,” filed on Aug. 6, 2020, and U.S. Provisional Application No. 63/184,970 titled “Intelligent System to Facilitate Treatment of Tissues and Calculi with Directed Energy,” filed on May 6, 2021, each of which is hereby incorporated by reference in its entirety.

### BACKGROUND

Directed energy (e.g., electromagnetic including optical, mechanical, acoustic including ultrasound, etc.) is increasingly a method of choice for treating various pathological conditions of the human body. One type of pathological condition that uses directed energy for treatment is urinary stone disease, including kidney and bladder stone, which is estimated to affect 12% of the world population. While most patients with kidney stone disease can pass stones naturally, severe cases of kidney stone disease (e.g., in which the patient cannot pass the kidney stone) requires medical intervention including use of directed energy. If severe cases of kidney stone disease are left untreated, extreme pain, nausea, vomiting, infection, blockage of urine flow, and loss of kidney function can soon follow.

Laser lithotripsy is one method of using directed energy to treat urinary stones, which uses directed laser energy, delivered via a fiber, to target the stone. Laser lithotripsy can be advantageous to other forms of directed energy (e.g., ultrasound) because laser light, during laser lithotripsy, can be delivered by a fiber, which is flexible enough to curve and traverse different hard to reach structures. In addition, small outer diameter of the fiber allows its insertion into working channels of most surgical instruments, including practically all scopes (rigid, semi-rigid, and flexible) used in urology. During laser lithotripsy, directed light energy from the laser is delivered to the stone, which breaks the stone into either finer particles that can be passed naturally, or bigger fragments that can be removed using auxiliary tools (e.g., baskets). Alternatively, the bigger fragments can be aspirated through a working channel of a scope (e.g., an endoscope).

Typically, during a laser lithotripsy procedure a practitioner (e.g., a doctor or surgeon) identifies a stone target by using a built-in-endoscope camera to receive images of the internal area of the patient. However, because the camera is the only device providing feedback to the practitioner, issues with the camera or during the image acquisition process (e.g., temporary obstructed camera's surgical field of view, camera's electronics malfunction, and the like) and variations in surgeon's reaction time can lead to inaccuracies, inefficiencies, errors, treatment time protraction/prolongation etc., in the lithotripsy procedure. Thus, it would be desirable to have improved systems and methods for controlling medical treatment processes.

### SUMMARY OF THE DISCLOSURE

Aspects and non-limiting examples are directed to methods and systems for performing laser surgical treatments.

In accordance with one embodiment, there is provided a method for controlling a surgical laser system that includes providing a surgical fiber configured to receive light reflected from a target in a surgical treatment area, and providing a computing device configured to couple with at least two photodetectors, each photodetector configured to detect an intensity of reflected light from the target in a different selected wavelength band, the computing device further configured to: receive the reflected light intensity in at least two selected wavelength bands, generate optical data corresponding to the reflected light intensity, and identify the target as a treatment target or a non-treatment target based at least in part on the optical data and a predetermined calibration based on at least two known targets.

In one example, the method further includes performing the predetermined calibration.

In a further example, at least one of generating the optical data and performing the calibration includes determining at least one ratio of a reflected light intensity of one selected wavelength band to a reflected light intensity of a different selected wavelength band.

In a further example, performing the predetermined calibration further includes: obtaining multiple reflected light intensity values from each known target of the at least two known targets, and establishing a threshold ratio value based at least in part on the multiple reflected light intensity values from each known target.

In another example, the method further includes determining a ratio value associated with a predetermined percentile for each known target based on the multiple reflected light intensity values from each known target, determining a difference value between a first ratio value associated with the predetermined percentile for a first known target and a second ratio value associated with the predetermined percentile for a second known target, comparing the difference value to a threshold difference value, and in response to a determination that the difference value meets or exceeds the threshold difference value, establishing the threshold ratio value based on the first ratio value and the second ratio value.

In a further example, the method further includes generating at least one histogram representation of values for each ratio of the at least one ratio, and determining the ratio value associated with the predetermined percentile for each known target based on the histogram.

In a further example, the method further includes determining the threshold difference value, wherein determining the threshold difference value comprises: comparing a first difference value associated with a histogram generated using a first ratio of the at least one ratio to a second difference value associated with a histogram generated using a second ratio of the at least one ratio. And determining whether the first difference value or the second difference value is larger, and in response to a determination that the first difference value is larger than the second difference value, selecting the first difference value as the threshold difference value, or in response to a determination that the second difference value is larger than the first difference value, selecting the second difference value as the threshold difference value.

In a further example, the method further includes assigning a weighting factor to the ratio associated with the largest difference value.

In one example, the threshold ratio value is based on an average of the first and second ratio values.

In one example, the predetermined percentile is the 80^{th} percentile.

In one example, generating the optical data includes determining the at least one ratio for the target in the surgical treatment area, and identifying the target comprises: comparing a ratio value of the at least one ratio for the target in the surgical treatment area to the threshold ratio value, and associating the target in the surgical treatment area with a known target of the at least two known targets based on the comparison.

In one example, the method further includes determining whether the known target is a treatment target, and in response to a determination that the known target is a treatment target, identifying the target as a treatment target, or in response to a determination that the known target is not a treatment target, identifying the target as a non-treatment target.

In a further example, determining whether the known target is a treatment target is performed in between every N laser pulses emitted by a treatment laser.

In one example determining whether the known target is a treatment target is performed after modifying a laser operating parameter of a treatment laser.

In one example, establishing the threshold ratio value further comprises defining a multidimensional decision space having n decision options based on at least two ratios of the at least one ratio, where n is the number of known targets.

In one example, the method further includes defining multidimensional threshold separation lines between the n decision options in the multidimensional decision space for discrimination between each target of the known targets.

In one example, the computing device is further configured to generate a control signal for controlling operation of a treatment laser based on the identification of the target. In a further example, the control signal includes activation, de-activation or an operating parameter setting for the treatment laser.

In one example the computing device is further configured to generate an audio, visual, or tactile signal to an operator based on the identification of the target.

In one example, the treatment target is a stone and the non-treatment target is tissue or a surgical component or a surgical treatment area medium.

In one example, the computing device is configured to couple with three photodetectors and the three different selected wavelength bands are selected from a group consisting of: about 400-410 nm, about 440-480 nm, about 460-480 nm, about 510-530 nm, about 540-560 nm, about 550-570 nm, about 570-580 nm, about 580-600 nm, about 600-620 nm, about 690-710 nm, about 740-760 nm, about 790-810 nm, about 920-940 nm, about 970-990 nm, and about 1150-1350 nm.

In one example, the computing device is further configured to identify the target as a treatment target or a non-treatment target based at least in part on a comparison against stored data from previously recorded reflected intensity values.

In one example, the computing device is further configured to identify the target as a treatment target or a non-treatment target based at least in part on a machine learning model.

In accordance with another exemplary embodiment, there is provided a surgical laser system that includes a surgical fiber configured to receive light reflected by a target in a surgical treatment area, and a computing device configured to couple with at least two photodetectors, each photodetector configured to detect an intensity of reflected light from the target in a difference selected wavelength band, and configured to: receive the reflected light intensity in at least two selected wavelength bands, generate optical data corresponding to the reflected light intensity, and identify the target as a treatment target or a non-treatment target based at least in part on the optical data and a predetermined calibration based on at least two known targets.

In one example, the computing device is further configured to perform the calibration.

In a further example, at least one of generating the optical data and performing the calibration includes determining at least one ratio of a reflected light intensity of one selected wavelength band to a reflected light intensity of a different selected wavelength band.

In one example, performing the predetermined calibration further comprises: obtaining multiple reflected light intensity values from each known target of the at least two known targets, and establishing a threshold ratio value based at least in part on the multiple reflected light intensity values from each known target.

In one example, performing the predetermined calibration further includes determining a ratio value associated with a predetermined percentile for each known target based on the multiple reflected light intensity values from each known target, determining a difference value between a first ratio value associated with the predetermined percentile for a first known target and a second ratio value associated with the predetermined percentile for a second known target, comparing the difference value to a threshold difference value, and in response to a determination that the difference value meets or exceeds the threshold difference value, establishing the threshold ratio value based on the first ratio value and the second ratio value.

In one example, performing the predetermined calibration further includes generating at least one histogram representation of values for each ratio of the at least one ratio, and determining the ratio value associated with the predetermined percentile for each known target based on the histogram.

In none example, the computing device is further configured to determine the threshold difference value, and determining the threshold difference value comprises: comparing a first difference value associated with a histogram generated using a first ratio of the at least one ratio to a second difference value associated with a histogram generated using a second ratio of the at least one ratio, and determining whether the first difference value or the second difference value is larger, and in response to a determination that the first difference value is larger than the second difference value, selecting the first difference value as the threshold difference value, or in response to a determination that the second difference value is larger than the first difference value, selecting the second difference value as the threshold difference value.

In one example, generating the optical data includes determining the at least one ratio for the target in the surgical treatment area, and identifying the target comprises: comparing a ratio value of the at least one ratio for the target in the surgical treatment area to the threshold ratio value, and associating the target in the surgical treatment area with a known target of the at least two known targets based on the comparison.

In one example, the computing device is further configured to determine whether the known target is a treatment target, and in response to a determination that the known target is a treatment target, identifying the target as a treatment target, or in response to a determination that the known target is not a treatment target, identifying the target as a non-treatment target.

In one example, the light reflected from the target is broadband light and the different selected wavelength bands include wavelength bands selected from the list consisting of: about 400-410 nm, about 440-480 nm, about 460-480 nm, about 510-530 nm, about 540-560 nm, about 550-570 nm, about 570-580 nm, about 580-600 nm, about 600-620 nm, about 690-710 nm, about 740-760 nm, about 790-810 nm, about 920-940 nm, about 970-990 nm, and about 1150-1350 nm.

In one example, the surgical system further includes a treatment laser, and the computing device is further configured to generate a control signal for controlling operation of the treatment laser based on the identification of the target.

In one example, the treatment target is a stone and the non-treatment target is tissue or a surgical component or a surgical treatment area medium.

Still other aspects, non-limiting examples, and advantages of these example aspects and non-limiting examples, are discussed in detail below. Moreover, it is to be understood that both the foregoing information and the following detailed description are merely illustrative examples of various aspects and non-limiting examples, and are intended to provide an overview or framework for understanding the nature and character of the claimed aspects and non-limiting examples. Non-limiting examples disclosed herein may be combined with other non-limiting examples, and references to “an non-limiting example,” “an example,” “some non-limiting examples,” “some examples,” “an alternate non-limiting example,” “various non-limiting examples,” “one non-limiting example,” “at least one non-limiting example,” “this and other non-limiting examples,” “certain non-limiting examples,” or the like are not necessarily mutually exclusive and are intended to indicate that a particular feature, structure, or characteristic described may be included in at least one non-limiting example. The appearances of such terms herein are not necessarily all referring to the same non-limiting example.

The foregoing and other aspects and advantages of the present disclosure will appear from the following description. In the description, reference is made to the accompanying drawings that form a part hereof, and in which there is shown by way of illustration one or more exemplary versions. These versions do not necessarily represent the full scope of the disclosure.

### BRIEF DESCRIPTION OF THE DRAWINGS

The following drawings are provided to help illustrate various features of non-limiting examples of the disclosure, and are not intended to limit the scope of the disclosure or exclude alternative implementations.

FIG. 1 is a schematic illustration of a non-limiting example of a smart laser system in accordance with aspects of the present disclosure.

FIG. 2 is a schematic illustration of an integrated laser-surgical system with the smart laser system of FIG. 1 being implemented in a surgical environment.

FIG. 3 is a schematic illustration of the optical adapter of FIG. 2.

FIG. 4 is a schematic illustration of the non-limiting example of the smart laser system of FIG. 1, further illustrating additional or optional components of the system.

FIG. 5 is a schematic illustration of another laser system. FIG. 5 also shows schematic representation of an optical adapter in accordance with aspects of the disclosure.

FIG. 6 is a schematic illustration of another laser system. FIG. 6 also shows a schematic representation of another example of an optical adapter in accordance with aspects of the disclosure.

FIG. 7 is a schematic illustration of another laser system. FIG. 7 is a schematic representation of yet another example of an optical adapter in accordance with aspects of the disclosure.

FIG. 8 is a cross-sectional view of the multicore fiber, and light(s) sources, light detector(s), that interact therewith.

FIG. 9 is a schematic representation of a smart laser system that utilizes fluorescent emission in accordance with aspects of the disclosure.

FIG. 10 is fluorescence responses of a soft human tissue (mucosa) and a stone (calcium oxalate monophosphate) in accordance with one or more aspects of the disclosure.

FIG. 11 is graphs of the autofluorescence response (e.g., the fluorescent light spectrum) for three different types of target materials (e.g., calcium oxylate monohydrate, struvite and xanthine).

FIG. 12 is a schematic of another example of a laser system.

FIG. 13 is a table outlining certain functionalities of an optical adapter.

FIG. 14 is a table of different examples of the types of features and functions provided by a laser system in accordance with aspects of the disclosure.

FIG. 15A is a flowchart of a process for determining that treatment target is a target material or is tissue.

FIG. 15B is a graph showing one example of an optical data profile for use in a laser treatment in accordance with the present disclosure.

FIG. 16 is a graph showing examples of reflection spectrum of LED for different treatment targets normalized to maximal level in accordance with aspects of the disclosure.

FIG. 17 is a graph showing examples of spectrums LED light reflected spectrum of LED non-normalized to spectra of LED for different targets in accordance with aspects of the disclosure.

FIG. 18 is a table of total (integrated) value of reflected light of LED in specific spectral ranges in signal for different targets in accordance with aspects of the disclosure.

FIG. 19 is a graph showing examples of spectrum of ratio of stone/tissue for different treatment targets in accordance with aspects of the disclosure.

FIG. 20 is a flowchart of a process for determining that treatment target is a target material or is tissue.

FIG. 21 is a flowchart of a process for determining a distance between a distal end of a fiber and a treatment target.

FIG. 22 is a further flowchart to FIG. 21 of a process for determining a distance between a distal end of a fiber and a treatment target.

FIG. 23 is a schematic of a calibration routine in accordance with aspects of the disclosure. The upper portion of FIG. 23 is a schematic of a calibration routine with pulsed probing or pilot beam source in accordance with aspects of the disclosure. The lower portion of FIG. 23 is a schematic of a calibration routine with continuous wave probing source or pilot beam in accordance with aspects of the disclosure.

FIG. 24 is a graph showing a relationship between a contact coefficient value and a contact distance between the tip of the fiber and a target in accordance with aspects of the disclosure.

FIG. 25 is a graph showing a relationship between the derivative of a contact coefficient and a contact distance between the tip of the fiber and a target in accordance with aspects of the disclosure.

FIG. 26 is a graph showing the relationship between a contact coefficient and different types of stone and soft tissue material in accordance with aspects of the disclosure.

FIG. 27 is a flowchart of a process for determining a presence of plasma within a treatment region.

FIG. 28 is a graph that exemplifies a spectral response from two different physiological materials in accordance with aspects of the disclosure.

FIG. 29 is another graph that exemplifies a spectral response from two different physiological materials in accordance with aspects of the disclosure.

FIG. 30 is a flowchart of a process for determining a temperature for a component of a laser system, or a treatment region.

FIG. 31 is a flowchart of a process for determining a presence of a bubble within a treatment region.

FIG. 32 is a graph showing pulse energy characteristics for different portions of pulsed laser radiation in ablation and coagulation applications, respectively, in accordance with aspects of the disclosure.

FIG. 33 is another graph showing pulse energy characteristics for different portions of pulsed laser radiation in ablation and coagulation applications, respectively, in accordance with aspects of the disclosure.

FIG. 34 is a flowchart of a process for detecting a problem with light transmission through the fiber.

FIG. 35 is another example of a functional schematic of another laser system.

FIG. 36 is a flowchart of a process for determining a distance between a distal end of a fiber and a distal end of a medical scope.

FIG. 37 is a schematic diagram showing different surgical fiber tip positions relatively to endoscope's distal end in accordance with aspects of the disclosure.

FIG. 38 is a graph showing the relationship between an LED signal measured by the laser system and the position of the distal end of the fiber in an endoscope in accordance with aspects of the disclosure.

FIG. 39 is a schematic illustration of a non-limiting example of a smart laser system in accordance with aspects of the present disclosure.

FIG. 40a is a flowchart of one example for a process of an in-patient calibration sequence in accordance with aspects of the disclosure.

FIG. 40b is a flowchart of one example of a lithotripsy procedure in accordance with aspects of the disclosure.

FIGS. 41a and 41b are histograms of two different examples of stone and soft tissue distinction in accordance with aspects of the disclosure.

FIG. 42 is a schematic of a time diagram of the sensor operation in accordance with aspects of the disclosure.

FIG. 43 is a schematic of one example of a two-dimensional decision space using two pairs of selected wavelengths in accordance with aspects of the disclosure.

FIG. 44 is a schematic of two histograms collected during calibration for two ratios in accordance with aspects of the disclosure.

FIG. 45 is a schematic explaining calculations used to determine variables using two ratios in accordance with aspects of the disclosure.

FIG. 46 is a table showing six example clinical cases performed on patients in accordance with aspects of the disclosure.

### DETAILED DESCRIPTION OF THE PRESENT DISCLOSURE

As described above, there are a variety of medical procedures or treatments that utilize lasers or photomedicine. One non-limiting example of a medical treatment is the optical or laser treatment of a target material using an optical fiber that is a surgical optical fiber. A target material may, in one non-limiting example, include a stone or calculus, or other material. The stone or calculus may be located in a bladder (e.g., often referred to as a bladder stone), located in the kidney (e.g., often be referred to as a kidney stone), located elsewhere in the renal or urinary system (e.g., referred to a urinary stone, a renal stone, or other stone), or may be located elsewhere. Additionally or alternatively, the target material may not be a stone or calculus or may not be associated with the renal or urinary system, but may be a material located elsewhere in the body or in other systems in the body. Examples of other relevant laser procedures include treatments of various urinary system pathologies such as benign prostate hyperplasia (BPH), bladder/prostate cancers, ureter strictures etc. In these procedures, laser energy is used for tissue ablation/vaporization, incision, coagulation and hemostasis. Furthermore, essentially equivalent procedures are used in other areas of surgery, such as, e.g., gastroenterology and laryngology.

Regardless of the particular clinical application or target material, it can be difficult for a medical practitioner to perform a medical procedure on a target material within a patient. For example, the field of view (“FOV”) of the camera may only be clear right before laser pulses are used on the target material, and thus the laser pulses may obscure the FOV of the camera (e.g., the laser pulses undesirably interacting with the imaging sensor of the camera thereby obstructing the clear representation of features in the image including the laser beam relative to the target material). Thus, only utilizing imaging data from the camera of the scope can provide non-ideal images of the surgical field, which can have spatial and temporal resolution limitations. In addition, even assuming that the imaging data is completely clear, extensive practitioner skill, capability, and experience is required for conducting a safe and efficient treatment (e.g., the practitioner reacting to changing conditions during the treatment).

As another example, the interaction between the light from the laser and the target material can make it difficult to ensure that the laser light is actually being directed at the target material (and not healthy tissue) during all processes of the procedure. An ablation procedure via laser lithotripsy can be characterized by multiple processes including (1) the formation of water vapor bubbles (and a vapor channel) in front of the distal end of the fiber, (2) the overheating of water in the operational area due to absorption of laser energy, (3) the formation of craters on the target material (along with retropulsion impact) with eventually fragmentation and dusting of the target material (e.g., the stone).

During each of these processes stone fragments and bubbles (including water vapor) can track in all directions, which scatters the illumination and obstructs or otherwise obscures the view of the treatment area. In fact, as the procedure naturally progresses, the camera's FOV can become more and more clogged (e.g., polluted) and eventually it becomes very difficult to discern the target within the images from the camera. In these moments, some practitioners see two options. First, the practitioner can take a more liberal approach to treatment and direct the laser at targets believed to be the target material (or a particle of the target material). However, because the practitioner's “view” is obscured (e.g., the FOV of the camera is significantly obscured), the risk of collateral damage to soft tissue (e.g., inadvertently directing laser light at non-treatment targets including healthy tissue) can increase significantly, and the risk of breaking the fiber can also increase significantly (e.g., due to mechanical pressure to a stone). Second, the practitioner can take a more conservative approach to treatment, which can involve the practitioner stopping firing, decreasing the laser power, temporarily increasing the irrigation fluid flow (e.g., for purposes of clearing the camera's FOV), and the like. However, unwanted results of this conservative approach can include prolonging the treatment time, prolonging the anesthesia time (which can be crucial in some cases), and limiting the practitioner's ability to finish treatment in one session thereby significantly increasing the cost of treatment (e.g., requiring multiple treatment sessions). Regardless of the treatment approach, major complications with soft tissue damage (even though being quite rare and arising in just less than 1% of the interventions) still occur and can be quite serious. In fact, the most severe cases can lead to perforation of the kidney or ureter wall, eventually requiring unwanted urgent kidney or ureter surgery.

While ensuring appropriate targeting of the laser light to the treatment target is one critical facet of laser lithotripsy procedures (and other laser-based procedures to address the target material), there are other critical facets including those with respect to safety and efficiency. For example, during lithotripsy or surgical soft tissue procedures, including benign prostatic hyperplasia (BPH) treatment, bladder, ureter, and kidney tumor incision or vaporization there are multiple conditions within the operational area that affect the efficiency of tissue interaction and overall quality of the clinical outcome. Some of these conditions include (1) employing the use of proper laser operating parameters for a given target tissue or stone type, (2) correctly determining the distance to the tissue surface from the distal end of the surgical optical fiber (e.g., to ensure that the laser light is being appropriately directed at the treatment target), (3) ensuring that the quality of the distal end of the surgical optical fiber is maintained throughout the procedure (e.g., to ensure that the distal end of the surgical optical fiber is transparent enough to transmit the laser light to the treatment target), (4) ensuring that the laser light does not undesirably obscure the FOV and compromising the visibility for the practitioner (e.g., by detecting the presence of a plasma flame that blocks laser radiation), (5) ensuring that the temperature of the treatment region (or in other words the operational area) does not undesirably increase (e.g., which could damage healthy tissues within the treatment area), (6) detect the presence of a vapor bubble and control the growth of the vapor bubble for efficient treatment of the target material, (7) ensuring that the fiber is intact (e.g., undamaged) and is properly positioned in the scope, (8) ensuring that stone movement during laser ablation is minimal to maintain contact or quasi contact with target, (9) ensuring that accidental breakage of the fiber inside the scope does not lead to scope damage etc. While the ability to properly recognize and react to these conditions would greatly increase the outcomes of the treatment (e.g., efficiency, safety, etc.), it is currently difficult if not impossible with typical laser lithotripsy to even detect these conditions-let alone appropriately act on them. In other words, typical laser lithotripsy systems cannot even detect these conditions.

Some non-limiting examples of the disclosure provide advantages to these issues (and others) by providing improved systems and methods for treating particular target materials, including medical calculi or stones as well as soft-tissue pathologies. For example, some non-limiting examples of the disclosure provide systems and methods for detecting the conditions above (and others) and appropriately acting on them. In some cases, just detecting the conditions can greatly assist the practitioner in achieving safe and effective laser treatments by the practitioner (or a computing device) analyzing the conditions, and appropriately acting on them (e.g., by interpreting the diagnostic information obtained from the prospective treatment sites and surrounding areas).

In some non-limiting examples, a laser system (e.g., a computing device of the laser system) can determine a distance between a treatment target or target material (e.g., a kidney stone), and act accordingly based on the determined distance (e.g., changing the treatment laser parameters such as CW power, pulse peak power, pulse shape and pulsewidth, interval between pulses, pulse frequency and average power, stopping the laser light altogether, informing the operator about condition detected and the like). In this way, the targeting of the laser light to the target material can be made more efficient. For example, when the surgical optical fiber is not quite in contact with the target material and while the laser fires pulses, the ablation efficiency is lowered because more laser energy is spent on heating the water and this decreases the laser fluency (power density) on the treatment target. In other words, the laser light is focused less on a specific location on the target material already having the liquid water vaporized off, and is more distributed around areas surrounding the specific location, which includes liquid water thereby using the laser light to heat the water (rather than being directed at and fracturing the target material).

As another example, the formation of bubbles during the treatment process can push the stone further away from the fiber tip (e.g., termed retropulsion), which can inadvertently (and undesirably) increase the distance between the distal end of the fiber and the target material thereby causing inefficiencies in treatment. Regardless of the cause of undesirable changes in distances (or non-ideal distances), determining a distance (including ensuring a predetermined distance) between tissue and the distal end of the fiber can increase treatment efficiencies (and allow the laser system or the practitioner to act accordingly).

In some non-limiting examples, a laser system can distinguish between different types of stone, and can distinguish between stone and tissue (e.g., soft tissue). In this way, the laser system can determine if the treatment target is actually being targeted by the laser light, and can determine particular laser operation parameters that are tailored to the treatment target (e.g., different treatment targets can necessitate different laser operation parameters). For example, if the treatment target is stone, and the laser system determines that the laser light is tissue rather than stone, then the laser system (or a practitioner controlling the laser system) can act accordingly (e.g., turn of the laser, decrease the power of the laser, etc.). Alternatively, if the treatment target is stone, and the laser system determines that the laser light is targeting stone (rather than tissue), then the laser system (or a practitioner) can increase the power (frequency, pulse width, etc.) of the laser light with proper confidence that the laser light is being directed at the stone. As another example, the laser system can determine that the treatment target is target material or tissue, and if the treatment target is target material can determine a type of target material. In this way, the laser system (or a practitioner) can adjust the laser operation parameters (e.g., CW power, pulse peak power, pulse shape and pulsewidth, interval between pulses, pulse frequency and average power of the laser light), based on the determination of the target material (or tissue) and type thereof. For example, different types of stone have different material properties (e.g., having a different hardness), which can benefit from different laser operation parameters. In particular, harder stones can require a higher energy laser pulse, while softer stones can require a lower energy laser pulse. Regardless of the treatment target, the ability to discern between stone (and types thereof) and tissue can shorten the procedure time, and increase the treatment outcome.

In some non-limiting examples, the laser system can provide information about whether or not the fiber position is in contact with tissue and can optimize the laser light and tissue interaction mechanism for the desired effect (e.g., using the mechanical energy of the bubble (for example, for separation of prostate capsule and adenoma tissue), using thermo-mechanical energy released by the absorption of the laser light, using thermal energy released by absorption of laser energy, etc.). For example, the laser system can be used to discern different tissue types (e.g., a prostate capsule, an adenomas tissue, etc.), each of which can have different desired predetermined distances from the distal tip of the fiber to the treatment target (in this case tissue), and can have different desired predetermined distances than calculi. For example, tissues can have a predetermined distance between the distal tip of the fiber and the treatment target that is tissue can be within a range between 1 millimeter and 10 millimeters (e.g., which can facilitate a mechanical effect in the tissue). As another example, calculi (or tissues that are desired to be ablated or coagulated) can have a predetermined distance between the distal tip of the fiber and the treatment target that is within a range between 0 millimeter and 5 millimeters (e.g., which can facilitate thermal ablation, thermal-mechanical ablation, etc., which can be used for tissue vaporization, incision, coagulation, etc.).

In some non-limiting examples, a laser system can determine a position of the fiber tip to ensure proper fiber positioning within the scope (e.g., endoscope). For example, if the fiber is situated within the scope (e.g., behind position of the distal end of the scope) and the laser emits laser light, the laser light can damage the components of the scope. In addition, if the distal end of the fiber is situated too far away from a distal end of the scope (e.g., greater than 4 mm), then the fiber can be undesirably curved by mechanical pressure from the scope and damaged.

As noted, the scope may be any of a variety of surgical or other medical scopes, including specialized or special-purpose scopes. The scope may include an imaging system and/or camera to receive images of the internal area of the patient. Image data from the scope may be used by the integrated systems described herein for analysis, control, and/or user feedback.

In some non-limiting examples, a laser system can determine the integrity of the fiber. For example, a laser system can determine that the fiber is cracked, fractured, tarnished, broken, etc. As another example, the laser system can determine that the fiber is curved past a vector of curvature (e.g., that can indicate that the fiber cannot appropriately guide the laser light to be emitted out of the distal end of the fiber).

In some non-limiting examples, including when contact with tissue is desired, the laser system can create a bubble in a controlled manner. For example, the laser system can generate laser light that is a pre-pulse (e.g., not treatment laser light) to form a controlled bubble (and a vapor channel, or in other words a “Moses” channel). Correspondingly, the laser system can determine if (and when) a stone or tissue material is reached within this bubble (e.g., the vapor channel contacting the stone or other target). In this way, treatment laser light can be avoided until after the vapor channel contacts the stone thereby better controlling laser light delivery and reducing stone retropulsion displacement (e.g., improving visibility, improving targeting of the treatment target, improving treatment efficiency etc.). In some cases, this controlled bubble can also be made continuously at the distal end of the fiber (e.g., the bubble being present continually while treatment laser light is delivered), which can avoid uncontrolled vaporizing of other water within the treatment region and increase tissue ablation efficiency. In some configurations, such as with the controlled bubble made continuously at the distal end of the fiber, the laser system can control the laser such that treatment laser light is allowed only at times when the target tissue is detected (e.g., using the processes described herein), for example in a non-contact (popcorn) mode of stone fragment ablation.

In some non-limiting examples, utilizing light from sources other than the treatment laser can be advantageous in that the light from the other sources does not undesirably interact with tissues, stones, etc. For example, the light can advantageously have a lower power than the power of the laser treatment light from the treatment laser.

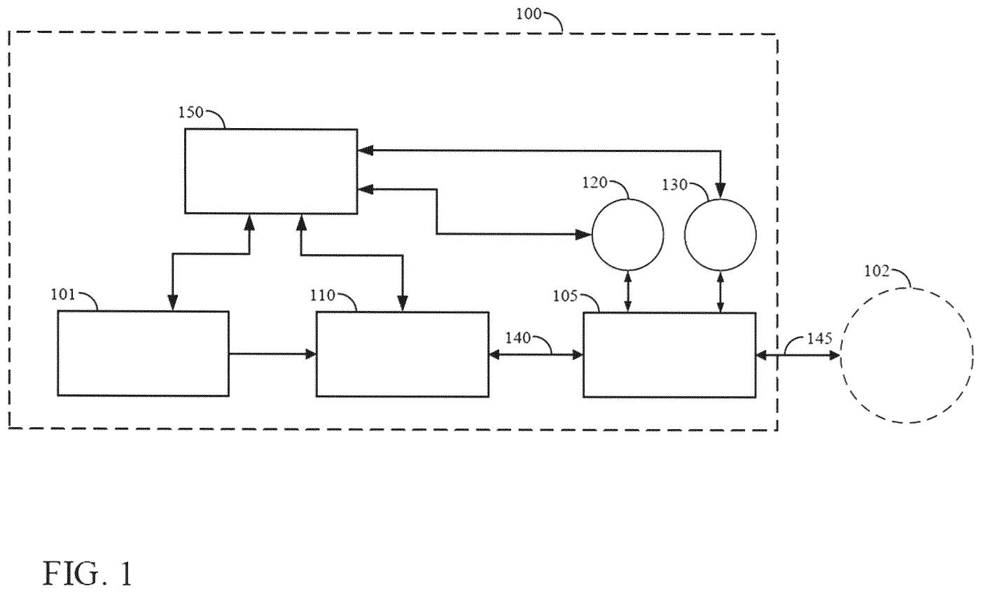

FIG. 1 is a schematic representation of a non-limiting example of a smart laser system 100 in accordance with aspects of the invention. As used herein, the term “smart” refers to the ability of one or more components of the laser system 100 to engage in two-way communication (i.e., transmit and/or receive a signal) with one or more other components of the system, such as a controller or control system of the laser system 150. For example, a control system (described in more detail below) can control other components of the system such as the laser driver 101 or laser source 110 (e.g., modify the laser operating parameters) in response to signals corresponding to the patient and/or treatment area that are transmitted to the control system via one or more sensors.

The system 100 comprises a multi-functional optical adapter 105, a laser source 110 to generate treatment radiation, a laser driver 101, a control system 150 that includes a processor for performing smart functionality, and surgical optical fiber 145 which can be part of the laser system or as a separate device. Laser driver 101 is a source of laser pumping current and voltage. For example, it can be the driver of a diode laser or a flash lamp. Diode lasers can be used for direct tissue treatment or for pumping solid-state or fiber lasers. Flash lamps can be used for pumping a solid state laser. The laser source 110 generates laser radiation, which is delivered to the optical adapter 105 via optical fiber or free beam 140. The laser radiation is partially reflected in the optical adapter 105 for laser power monitoring and then is coupled into the surgical optical fiber 145. The laser radiation from a distal end of the surgical optical fiber 145 interacts with a treatment target (i.e., treats tissue or stone) in the surgical treatment environment 102. The optical adapter 105 is also connected to sources of probe signals 130, e.g., a probe light source (also can be an excitation light source), and one or more sensors 120 of electromagnetic radiation. Probe signals from the source of probe signals 130 are also coupled into the surgical optical fiber 145 (via the optical adapter 105) and the returning probe signal (also referred to herein as probe signal data) as well as other electromagnetic radiation generated in the surgical treatment environment that may be partially deflected into the sensors 120 (via the optical adapter 105) for further reference and analysis.

During interaction of the surgical laser radiation with liquid in the treatment zone, biological tissue, stone, and/or surgical components, like a basket, certain electromagnetic signals are reflected or generated in response to excitation and can propagate through the surgical optical fiber 145 into the optical adapter 105, where they are further directed to particular sensors 120. These electromagnetic signals can include probe signal data based off the source of probe signals, which can be directed into the sensors 120 (via the optical adapter 105). In some instances, these electromagnetic signals are reflected from structural non-uniformities of the surgical optical fiber 145 or special structure of fiber distal end (e.g., Brag grating, fluorescence doped end or special form of the tip, such as conical, side firing and etc.). A control system 150 receives the signals from the sensors 120 and performs an analysis which is then used by the control system 150 to control other components of the system, such as the laser driver 101 and laser source 110. Laser sources 110 can be any laser with parameter(s) optimized for desired therapeutic effect and transmitive through surgical fiber 145. For example, for a urological procedure such as lithotripsy and using silica fiber it can be laser sources having a wavelength in the range of 1.85-2.2 μm, pulse energy 0.001 to 10 J, peak power 0.1 to 100 kW, and average power 2-200 W. It can be Ho:YAG, Tm:YAG, Tm:YLF and other solid state laser with such parameters with flash lamp or diode pumped. Another example is Tm fiber laser with diode pump in free-running or Q-switch modes of operation. This laser can also be used for soft tissue procedures. In addition, lasers with wavelengths of 400-600 nm can be used. A diode laser, for example, with a wavelength of 400-460 nm, or 780-1100 nm, or 1300-2100 nm, or second harmonic of Nd:YAG laser with a wavelength of 530 nm can be used. Such a laser can work with in continuous wave (CW) mode with power 10-300 W. A diode or diode-pumped laser, such as a fiber laser source, can also be preferable for some configurations.

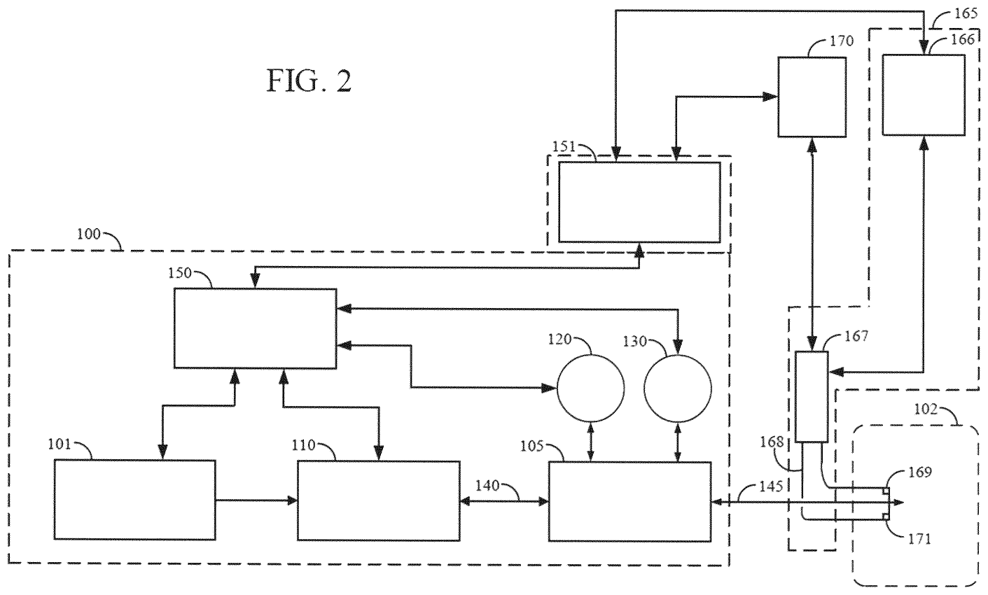

In another aspect of the invention, the smart laser may be a part of an integrated treatment system (FIG. 2), which, in addition to the laser system 100 and surgical fiber 145, may include a scope (flexible, semi-flexible, or rigid) 167, an aspiration, irrigation or aspiration/irrigation sub-system 170, and an Artificial Intelligence (AI)-run control center 151. The AI control center performs the initial processing of signals from imaging system 166 of the scope 167, the aspiration/irrigation sub-system 170 and implements synchronization with laser system 110 and laser driver 101. Scope 167 includes a handle, rigid, semi-rigid, or flexible shaft 168 with imaging sensor 171 and illumination light sources like LED or lamp 169 (lamp with fiber delivery) with illumination emission from the distal end of the shaft. AI-run control center 151 can be integrated with laser system controller 150, or to be a separate unit or integrated with scope imaging system 166 with initial processing and control of video sensor 171. Such integrated system will greatly extend benefits of the smart laser for both patient and operator.

For example, in accordance with one embodiment, the smart laser may receive and process visual information received by the video camera of the scope 169. This information can be used stand-alone or in combination with other informational channels available to the system from sensors 120 (elastic scattering, fluorescence etc.) and information about laser parameters from controller 150. This information can be used for detection and recognition of various treatment conditions such as: 1) detecting/distinguishing between soft tissue and stone, 2) recognition of stone type and stone substructure, 3) recognition of soft tissue type (e.g., capsule/adenomas tissue boundary, detecting/distinguishing a tumor and normal tissue), 4) the distance between tissue or stone and the fiber distal end, 5) tissue bleeding, 6) surgical field visualization quality which can be compromised by scattering of light on products of ablation, 7) stone retropulsion displacement, 8) popcorning performance, 9) distal tip damage or contamination, 10) flashing in treatment area, 11) treatment organ recognition.

The camera image can be further processed by the image processor 166 or the AI-run control center. The image processing algorithm is developed, optimized and validated for each clinical embodiment using analysis of clinical endoscopic video imaging and machine-learning methodology.

Use of information from imaging system of endoscope 166 will further increase accuracy of measuring a distance to the target, differentiating between stones and soft tissues, and identifying stone or tissue type. When the LED of the scope is used for target illumination, the LED spectrum will become available to the control center in real time, to ensure accurate dynamic normalization of the elastic/fluorescence spectra.

In another embodiment, the aspiration/irrigation sub-system can be equipped with a suite of pressure/flow sensors and thermal sensors that will transmit their readings to the AI run control center 151. This information can be used for: 1) measuring temperature of aspiration and irrigation fluid to prevent overheating and damage to tissue epithelium and deeper layers; 2) measuring liquid flow rate to calculate temperature inside treatment organ, which is directly proportional to the laser power and inversely proportional to the liquid flow rate, 3) measuring irrigation or aspiration pressure and its difference to prevent treatment organ damage due to excessively high positive or low negative pressure. Firing the laser can then be synchronized with irrigation and/or aspiration pump flow and pressure rate through signaling from AI-run control center to achieve maximal clinical outcome and safety. For example irrigation and aspiration pump can work in pulse mode and synchronized with laser pulsing to achieve maximal clean view of surgical target by pulsing irrigation and improve aspiration rate with keeping safe margin.

Real time information from the imaging system 166 and from the sensor 120 can be combined and processed in the laser control system or AI-run control system in real time to increase accuracy and for redundancy purposes. For example, the distance between stone or tissue and the distal fiber end can be measured by the back refection signal of endoscopic LED (see “Using integrated signal” Section) or by processing the image of the endoscopic video system. A command for enabling or disabling laser emission or changing of laser parameters in predefined ranges can be issued if both signals are in acceptable ranges.

Smart laser system (FIG. 1) or smart laser system integrated with endoscope and other devices (FIG. 2) in the proposed invention is designed to operate in the following steps. 1^{st} step is obtaining signal for surgical treatment environment 102 using surgical fiber 145 and sensors 120 or/and signals from endoscopic imaging system 166 and other devices such as irrigation/aspiration system 170. 2^{nd} step is processing this signal to provide information about surgical treatment environment or surgical fiber and instruments in surgical treatment environment. 3^{rd} step is to send signal from laser system control unit to the laser driver to automatically change pumping current and/or voltage to adjust laser pulse power, temporal profile and peak power, laser energy, interval between laser pulses and repetition rate and average laser power to achieve necessary clinical outcome. 4^{th} step is to evaluate desired clinical outcome. 3^{rd} step can be, in a particular case, full interruption of laser energy delivery. In some embodiments, 3^{rd} step can result in generating audio or visual signal to operator with optional coding of visual signal color and/or intensity of audio signal with different tone and intensity to request laser parameter changing by operator. Step 4 is the assessment of the achieved clinical outcome and decision whether to stop or to continue treatment.

Table 1 below is a list of non-limiting examples of the types of features and functions provided by the disclosed systems and methods.

TABLE 1
Examples of Functionality

| System Feature | Function | Benefit |
|---|---|---|
| Fiber breakage | Detects fiber breakage | Prevents laser emission if the distal end |
| sensor | | has broken at fiber connector, in space |
| | | between laser and instrument (scope), |
| | | inside scope, and/or in patient's body |
| Fiber position in | Detects position of fiber tip | Prevents laser emission inside of the |
| scope | (inside/outside) of scope | scope |
| Detection of | Allows laser emission only | Prevents water overheating and |
| contact with stone, | when the fiber tip is close | unintended firing on soft tissue, reduces |
| and hard or soft | to a predefined distance | retropulsion and more efficient |
| tissue | stone or tissue | popcoming, and distinguishes between |
| | | contact with stone or soft tissue to |
| | | protect soft tissue from unwanted |
| | | damage (perforation, ablation, or |
| | | coagulation) |
| Detection of stone | Recognize stone type | Automatic control of laser setting to |
| type | | increase the speed of ablation |
| Detection of soft | Recognize tissue type and | Improves safety and precision of BPH, |
| tissue type | status (including prostate | bladder cancer, and other soft tissue |
| | capsule, and benign and | treatments |
| | malignant lesions) | |
| “Plasma” emission | Measure high temperature | Minimizes fiber damage, prevents |
| sensor | (45-2500° C.) heat | tissue carbonization, basket damage, |
| | emission from | and image distortion |
| | contaminated fiber tip, | |
| | carbonized tissue, | |
| | overheated stone, or basket | |
| Maintain ambient | Measure low temperature | Increases the safety of the treatment |
| medium temperature | heat emission (40-95° C.) | |
| Bubble formation | Optical detection of | Increases the speed of the treatment due |
| detector | air/water interface | to adaptive control of vaporization |
| | | channel (Moses effect) and delivering |
| | | ablation or coagulation energy when a |
| | | vapor channel is created between the |
| | | end of the fiber and the stone or tissue |

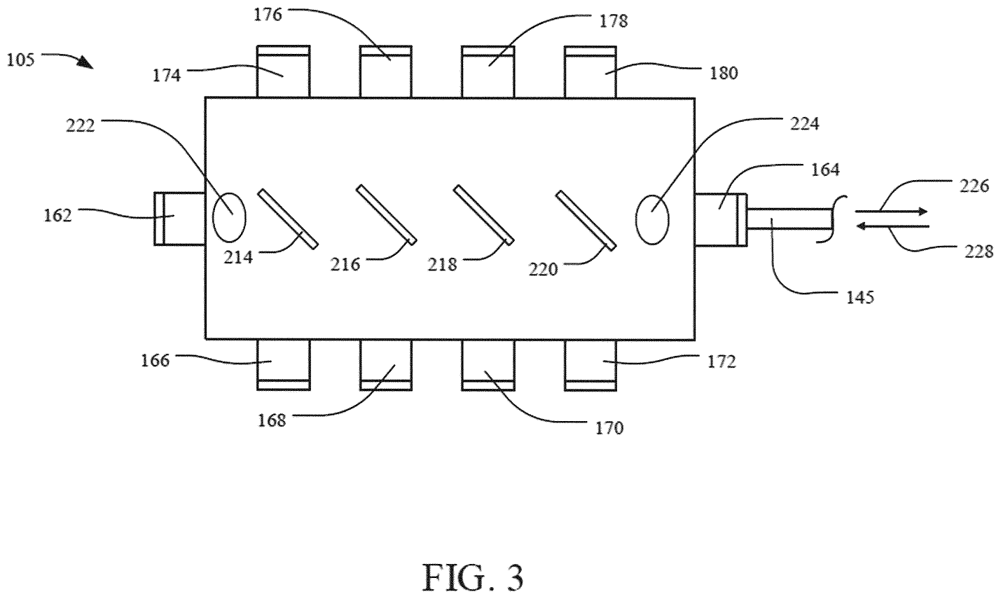

FIG. 3 shows a schematic illustration of the optical adapter 105, which is one example of the optical components of the optical adapter 105 that can facilitate directing light to and from different ports. For example, the optical adapter 105 can include beamsplitters 214, 216, 218, 220, and lenses 222, 224. Each of the beamsplitters 214, 216, 218, 220 can be positioned within the optical adapter 105 (e.g., the housing of the optical adapter 105) and each can be oriented in the same way (e.g., angled as illustrated in FIG. 3). The angle between the axis of laser beam and normal to the beamsplitter surface can be in the range 10 to 70 degrees, preferably for some applications, 30 to 50 degrees. However, while there are four beamsplitters illustrated, other numbers of beamsplitters can be used, especially, for example, when there are a different number of pairs of ports. Thus, in some cases, the number of beamsplitters can match the number of aligned ports of the optical adapter 105 (e.g., with the exception of the ports 162, 164). In addition, while all the beamsplitters 214, 216, 218, 220 are illustrated as being oriented in the same way, it should be appreciated that the beamsplitters 214, 216, 218, 220 can be oriented in different ways, with the directing of light between ports being changed by the orientation of the beamsplitter. Each beamsplitter can have dielectric coatings with maximal transmission of the laser beam and optimal reflection in spectral ranges of the probing beam and back reflection signals associated with ports connected to that beamsplitter. In addition, some ports can include a lens or sets of lenses to re-image the proximal end of the surgical fiber 159 onto the detector.

As shown in FIG. 3, a beamsplitter 214 can be positioned between (and aligned with) ports 166, 174, another beamsplitter 216 can be positioned between (and aligned with) ports 168, 176, another beamsplitter 218 can be positioned between (and aligned with) ports 170, 178, further beamsplitter 220 can be positioned between (and aligned with) ports 172, 180, and each of the beamsplitters 214, 216, 218, 220 can be positioned between (and aligned with) the ports 162, 164. Each of the beamsplitters 214, 216, 218, 220 can direct light into (and out of) respective ports 174, 176, 178, 180, and each is able to transmit laser light therethrough to the optical port 164 (and to the surgical fiber 145). For example, light can be emitted into the port 174, directed by the beamsplitter 214 through the optical port 164 and into the proximal end of the fiber 145, which can follow in the direction 226 (e.g., which can extend from the proximal end to the distal end of the fiber 145). As another example, light that is directed into the distal end of the fiber 145 along the direction 228 (e.g., which can extend from the distal end to the proximal end of the surgical fiber 145) can be emitted through the port 164, can pass through the lens 224, and can be directed by the beamsplitter 220 through the port 180.

In some non-limiting examples, the lens 222 can be in optical communication with the treatment laser 152, and can be positioned in front of the port 162 within the optical adapter 105 behind each of the beamsplitters 214, 216, 218, 220. In some cases, the lens 222 can be a collimating lens. In this way, laser light can be collimated after passing through the lens 222. In some non-limiting examples, the lens 224 can be a focusing lens, which can focus light that passes through the focusing lens in the direction 226, and can diverge light that passes through the focusing lens in the direction 228. In some cases, the lens 224 can be positioned behind the port 164 and in front of each of the beamsplitters 214, 216, 218, 220 within the optical adapter 105.

In some non-limiting examples, the lens 222 can be in optical communication with the treatment laser described above. In some cases, the lens 222 can be a collimating lens. In this way, laser treatment light can be collimated after passing through the lens 222. In some non-limiting examples, the lens 224 can be a focusing lens, which can focus light that passes through the focusing lens in the direction 226, and can diverge light that passes through the focusing lens in the direction 228. In some cases, the lens 224 can be positioned behind the port 164 and in front of each of the beamsplitters 214, 216, 218, 220 within the optical adapter 105.

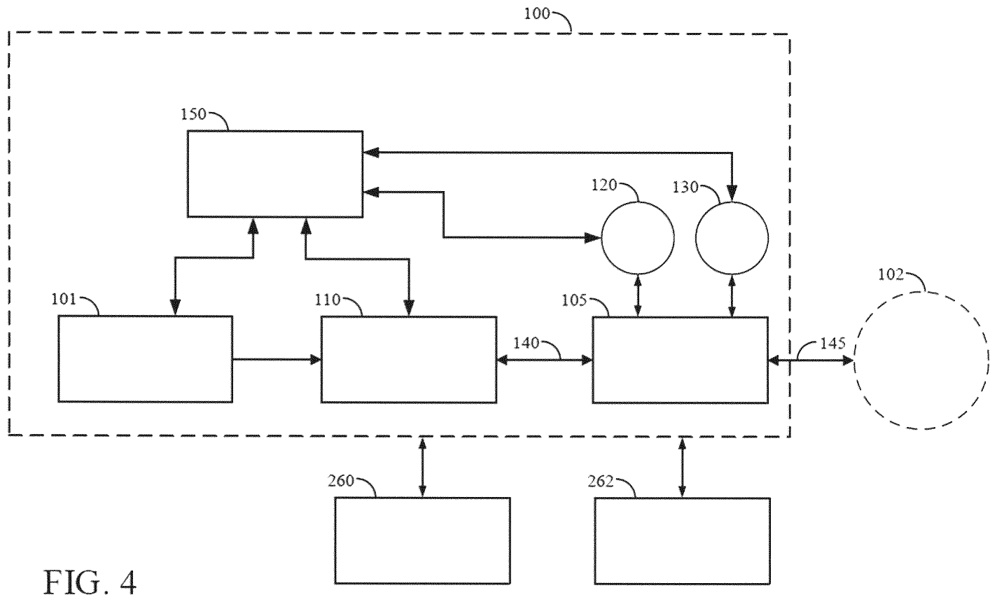

FIG. 4 shows the schematic illustration of FIG. 1, but further includes an input device 262 and an output device 260. That is, the system described above with respect to FIG. 1 may be adapted to include a variety of user interfaces, such as a display that may form the output device 260 and a variety of user controls or input devices that can form the user input 262.

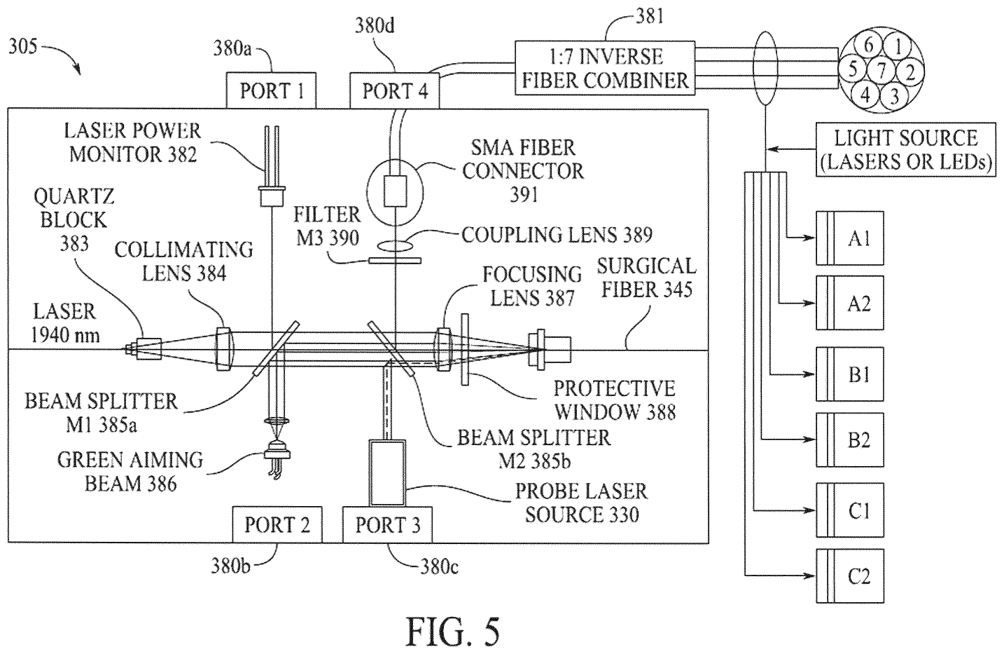

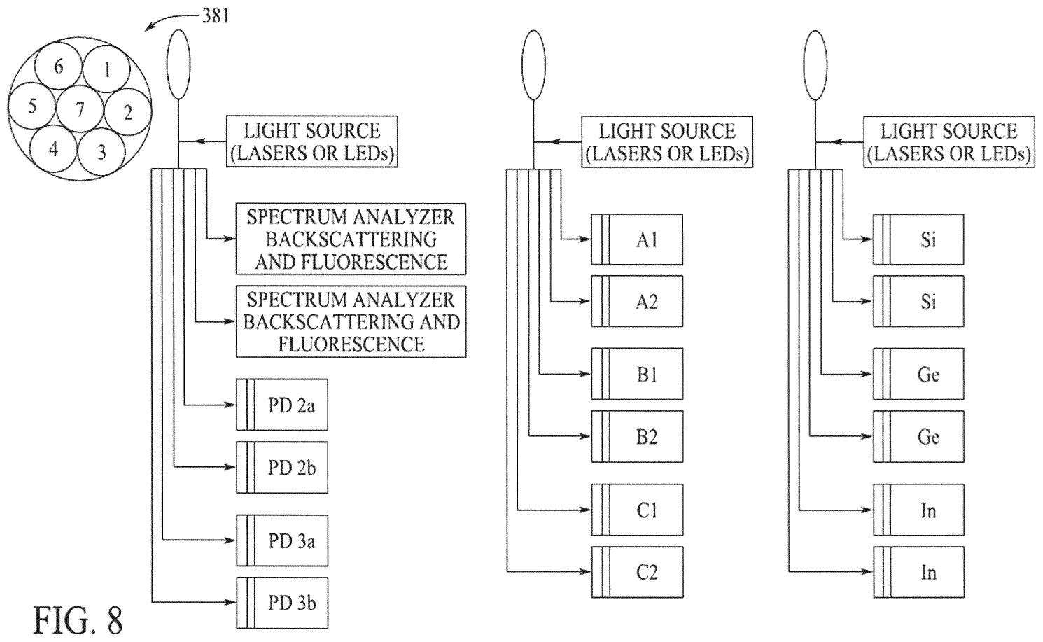

FIG. 5 shows a schematic illustration of a laser system 300, which can be a specific implementation of the laser system described above, or others described herein. The laser system 300 can include an optical adapter 305 (which can also be referred to as an optical coupler, an optical module, etc.) as shown in FIG. 3. A non-limiting list of components or features shown in FIG. 3 that can be included in the optical adapter 305 include at least one port, an inverse fiber combiner 381 (also, illustrated in FIG. 8), a laser power monitor 382, a quartz block 383, a collimating lens 384, beamsplitters 385a, 385b, an aiming beam source 386, a focusing lens 387, a protective window 388, a coupling lens 389, a filter 390, and a fiber connector 391.

The optical adapter 305 can be a multi-functional component, where the optical adapter 305 can direct light along different optical paths. For example, the optical adapter 305 can direct the laser radiation from the laser source 310 into the surgical fiber 345 using lenses 384 and 387. In addition, the optical adapter 305 can direct light to one or more detectors to monitor a temperature of the distal end of the surgical fiber or liquid, back reflected from the target or non-target light, fluorescence light, other light, etc., each of which can be an indicator of proper working condition in treatment area. In some cases, a light source can emit a visible laser beam (e.g., green light as in FIG. 5) as an aiming beam into the surgical fiber 345 using the beamsplitter 385a. For example, the visible laser beam can be emitted towards the beamsplitter 385a, which can be directed by the beamsplitter 385a so that the visible laser beam is directed into the proximal end of the surgical fiber 345.

In some non-limiting examples, probe light from the source of probe light 330 can be directed into the proximal end of the surgical fiber 345 using a reflective prism or mirror. Light that is transmitted back through the surgical fiber 345 from the distal end and to (and out) the proximal end can be separated using a beamsplitter and additional beamsplitters (not shown), can be directed into a single or multicore fiber (e.g., using a coupling lens to direct the light into the fiber), and can deliver this light to one or more light detectors. In addition, in some non-limiting examples every light detector can be configured with a spectral filter to select a desired wavelength or wavelengths. In some non-limiting examples, one or more of the light detectors can be configured as a spectrometer to measure a spectral distribution of this light (e.g., back reflected light of the scope broad spectrum illumination light sources such as LED or fluorescent light heat radiation light).

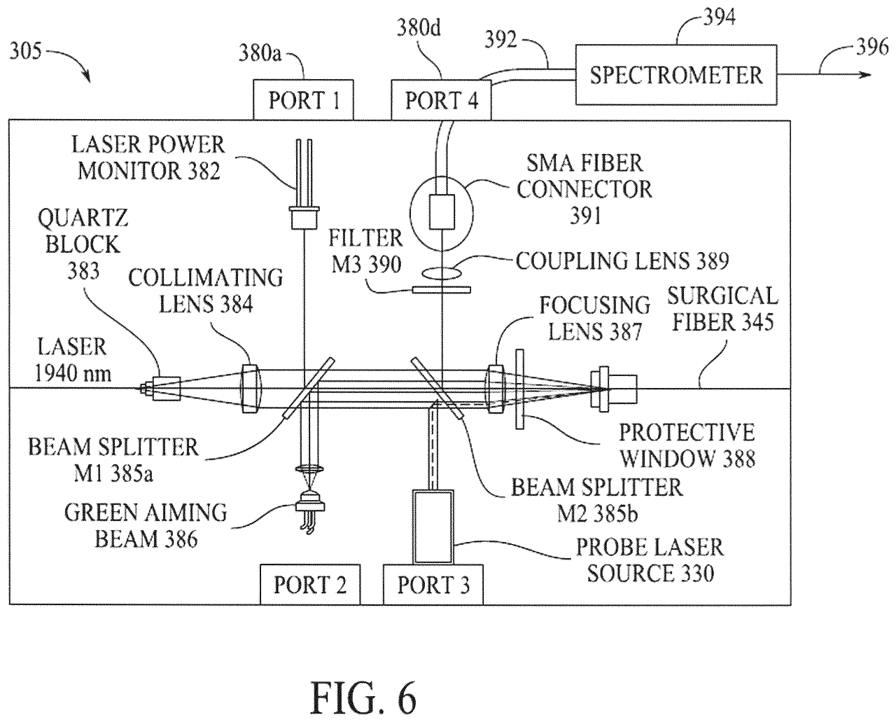

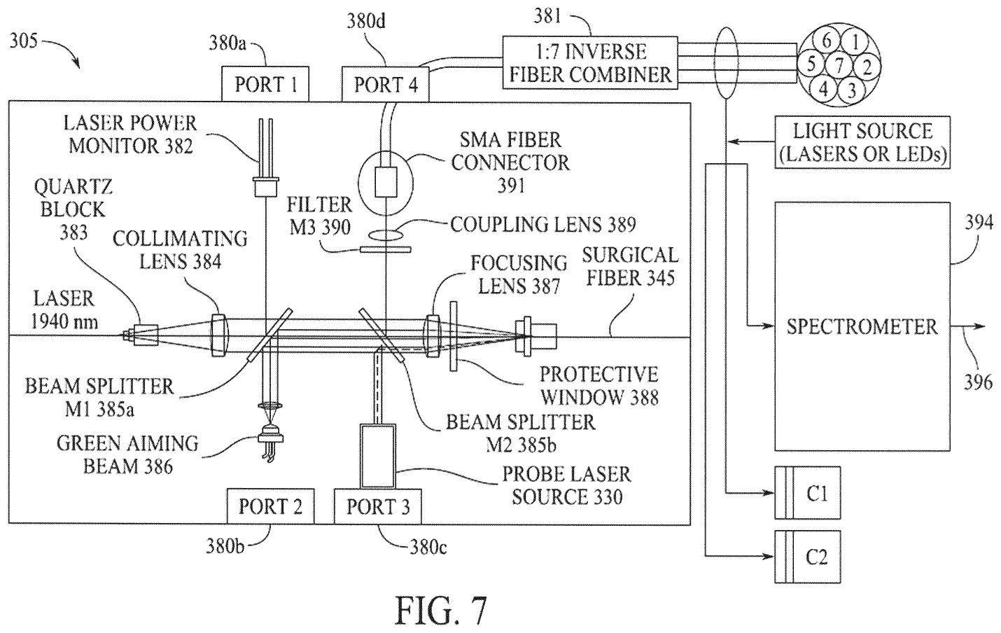

FIG. 6 shows a shows a schematic illustration of a laser system 301 while FIG. 7 shows a schematic illustration of a laser system 303, each of which can be a specific implementation of the laser system 100, 300 or others described herein. Each of the laser systems 301, 303 include the optical adapter 105 configured to direct light to a spectrometer. For example, light directed into the distal end of the surgical fiber 345 can be back reflected from the target or illumination light of scope reflected from the target or heat radiation for distal fiber end or heated treatment area using beam splitter 185b directed into a fiber 392 (see FIG. 6) and coupling into detector such as photodiode or spectrometer. Optical data from spectrometer are transferred into a computing devise through cable 396. In some cases, the light from treatment area reflected from beam splitter 385b coupling with lens 389 to fiber 380b, which input fiber of inverse fiber combiner 381. Some of 7 fibers of inverse fiber combiner 381 can be connected to spectrometer 394 and others into photodiodes. In some cases, this light that is directed into the distal end of surgical fiber 345 can be broad spectrum light (e.g., including multiple wavelengths within a range of 400 nm to 800 nm), which can originate from an endoscope illumination light (e.g., this light being reflected, scattered, etc., from tissue, stone, particles, surgical components in a liquid environment in the treatment area). In some cases, this light (e.g., that is delivered to the treatment area through the surgical fiber and reflected back from tissue, stone, particles in a liquid environment in the treatment area, surgical components, etc.) can be broad spectrum light originating from a probing light source that is configured to emit light having a wavelength within a wavelength range of 300-2700 nm. In some cases, this light can be broad spectrum light heat radiation light that is generated in the treatment region in response to laser light (e.g., high power laser radiation) interacting with tissue, stone, surgical components, a product of ablation created by the laser light interaction, or distal end of surgical fiber. In some configurations, this light can be fluorescent light induced by a probing laser source, a broad spectrum light source (e.g., an LED), an aiming beam, etc. In some cases, this light can be fluorescent light generated from additional molecules or particles (e.g., fluorescent markers) delivered to the patient (e.g., during treatment) by, for example, irrigation fluid or via injection into the blood stream. In some cases, this fluorescent light can be induced by a probing light source (e.g., a probing laser source, an LED, the aiming beam, etc.).

Regardless of the configuration, this light (e.g., broad spectrum light) can be directed to the spectrometer 394 thereby generating an optical spectrum, which can be received by a computing device. In some cases, this optical spectrum can be analyzed using a computing device or the spectrometer 394 itself (e.g., using signal processing electronics of the spectrometer 394). In some cases, the optical database on the spectrum can be received by a computing device via a cable 396, and can be used for purposes of generating audio, visual, or other signals (e.g., control commands) to the laser system, the user, or to determine (or modify) laser operation parameters, based on the information provided by the analysis of the optical spectrum.

In some non-limiting examples, the light (e.g., broad spectrum light) can be delivered to the spectrometer 394 by a fiber 192 (see FIG. 6), or via one or more cores of a multicore fiber 381 (see FIG. 7). In this non-limiting example, other cores of the multicore fiber 381 can be used for other signal detection (e.g., light detection). For example, other signal detection can include a probing light reflected from tissue, stone, surgical components, etc., and directed into the distal end of the surgical fiber 345, a fluorescent light (without spectral resolution) also directed into the distal end of the surgical fiber 345. In some cases, a central core of the multicore fiber 381 can be used to deliver light (e.g., monochromatic or broad spectrum light) into the treatment region. For example, a light source coupled in central fiber can travel through the coupling lens 189, can be directed by the beamsplitter 385b and lens 189 into the proximal end of the surgical fiber 345. In this case, a portion of this light can be back reflected light and can be used in a similar manner as broad spectrum endoscopic illumination light for detecting, distinguishing, etc., the type of tissue, stone, surgical components, product of ablation, and other treatment characteristics based on an analysis of the optical spectrum of back reflected (scattered) light and comparing it with the optical spectrum of the light originating from the light source coupled into central fiber.

In regard to the optical adapter 305, the fiber 381 can be split into multiple optical channels, and each channel being configured with a respective light detector (or light source). In some cases, functions responsible for critical operation can be split for redundancy to reduce the risk of malfunction (e.g., multiple optical channels sensing the same type of light, such as the same wavelength of light). This optical split can be further used to direct other probing light into the optical path defined by the surgical fiber 345. In some cases, a computing device can receive signals (e.g., data) from each optical detector, and each spectrometer, which can be analyzed for determining system parameters to improve the efficiency and safety of the treatment.

As shown in FIG. 7, the spectrometer 394 can be in optical communication with one or more of the optical channels of the multicore fiber 381, so that light from each optical channel can be directed to the spectrometer 394. In some cases, the multicore fiber 381 can include an inverse combiner (e.g., a tree coupler), which can combine light from each optical channel to be directed through the port 380d (and into the distal end of the surgical fiber 345). Correspondingly, light from the proximal end of the surgical fiber 345 can be directed into the inverse combiner and can be split (e.g., equally) among each of the optical channels of the multicore fiber 381.

FIG. 8 shows a cross-sectional view of the multicore fiber 381. As shown in FIG. 8, the multicore fiber 381 includes multiple optical channels (e.g., seven as illustrated), with each optical channel being associated with a light detector, or a light source. For example, in a first configuration, a first optical channel can be in optical communication with a spectrometer, a second optical channel can be in optical communication with the spectrometer (or a different spectrometer), and a third, fourth, fifth, and sixth optical channel can be in optical communication with a respective light detector. In some cases, the seventh optical channel can be in optical communication with a light source.

In a second configuration, each of the first, second, third, fourth, and sixth optical channels can be in optical communication with a respective light detector (or a respective light source). In some cases, the seventh optical channel can be in optical communication with a light source. In a third configuration, the first and second optical channels can be in optical communication with a first light detector, the third and fourth optical channels can be in optical communication with a second light detector, and the fifth and sixth optical channels can be in optical communication with a third light detector.

In some configurations, each optical channel of the multicore fiber 381 can be in optical communication with a respective light source, and a respective light detector. For example, each respective light source can emit light into the respective optical channel of the multicore fiber 318, while each light detector can receive light from the respective optical channel. In some configurations, each optical channel of the multicore fiber 318 can include a beamsplitter, each of which can facilitate directing light from a respective light source to a respective optical channel and receiving light from the respective optical channel and to the respective light detector. In some cases, this configuration having a light source and a light detector for each optical channel can reduce the number of ports needed for the optical adapter 305. In some cases, while the multicore fiber 381 is illustrated, the multicore fiber 381 can be substituted with multiple fibers. In this case, each of the multiple fibers corresponds to an optical channel of the multicore fiber 381.

In accordance with another non-limiting example, fluorescence emission from tissue and calculi can be used to identify the target type and select optimal treatment parameters. Either fluorescence of endogenous chromophores (autofluorescence) or that of exogenous chromophores (induced fluorescence) can be utilized for identification.

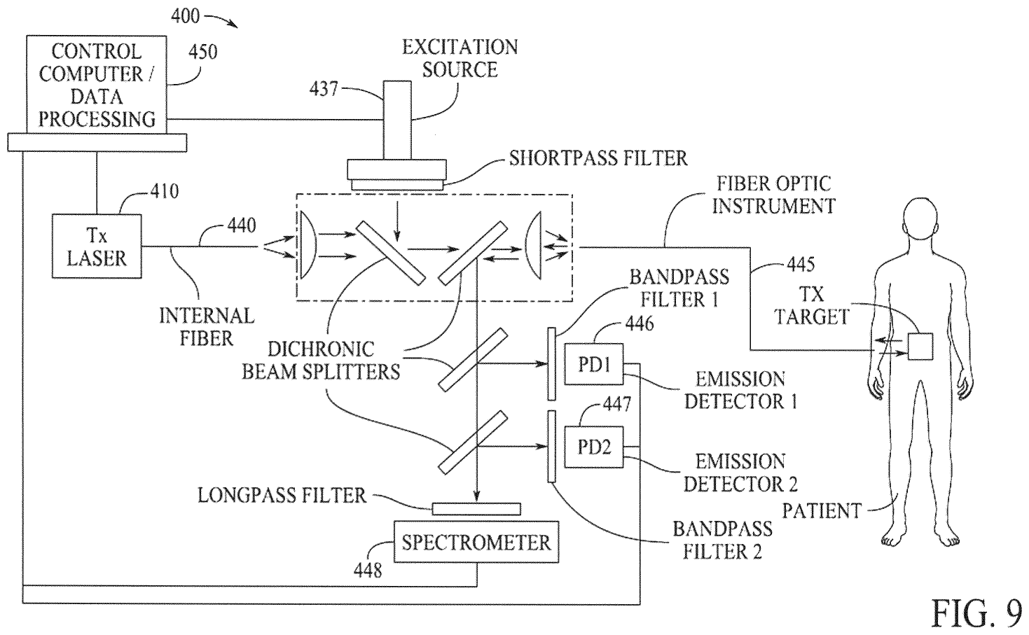

FIG. 9 is a schematic of one non-limiting example of a laser system 400 that uses the detection of fluorescence emission for purposes of control and treatment optimization. A fluorescent light source 437 (e.g., a laser or LED) (also referred to herein as a excitation light source) serves as a probe light source and is used for excitation of fluorescence. For autofluorescence non-limiting examples, a preferable range of excitation wavelengths for excitation light is from 290 nm to 900 nm, more preferably from 330 to 700 nm, and even more preferably from 360 nm to 400 nm. For exogenous-fluorescence non-limiting examples, the excitation wavelength is selected based on the fluorophore used.

The excitation light from the excitation source 437 can be directed into the fiber optic instrument, e.g., fiber, and preferably this is the same fiber that transmits the treatment laser light from the laser source 410. Feedback light from the fluorescence emission can be transmitted through the same fiber 445 to detectors 446, 447, and a spectrometer 448. At least one (preferably, two or more) detectors, e.g., 446, 447 can be used to detect fluorescent light at different wavelength bands. In some cases, one or more of the detectors can be PIN photodiodes, APD photodiodes, photomultipliers etc. The control system 450 may include additional components for facilitating accurate signal acquisition, such as lock-in amplifiers, heterodyning electronics, etc. Some non-limiting examples may include use of spectrometer to collect full spectral fluorescent information. For example, the spectrometer 448 can receive light that is directed back through the distal end of the fiber 445 and emitted out of the proximal end of the fiber 445.

In some non-limiting examples, the laser source 410 can emit continuous wave (CW) light. In CW light non-limiting examples, the target identification is performed through at least one of (1) or (2). For (1), the ratio of the fluorescence signal to the reflected excitation signal can be used. For example, the ratio of the fluorescence signal F to the reflected signal R (F/R) is expected to be higher for stones than for soft tissue, and if the ratio F/R exceeds a predetermined limit (e.g., 10) or desired range, then this signifies that the target is stone and not soft tissue (and vice versa). For (2), the ratio of two or more fluorescent signals measured at different wavelength bands can be used (e.g., a ratio of the fluorescence signal F1 at 400-500 nm band to the fluorescent signal F2 at 550-650 nm band). For example, the ratio of the fluorescence signal F1 to the fluorescent signal F2 is expected to be lower for stones than for soft tissue and if F1/F2 is below a predetermined limit or range, then this signifies that the target is stone and not soft tissue (and vice versa).

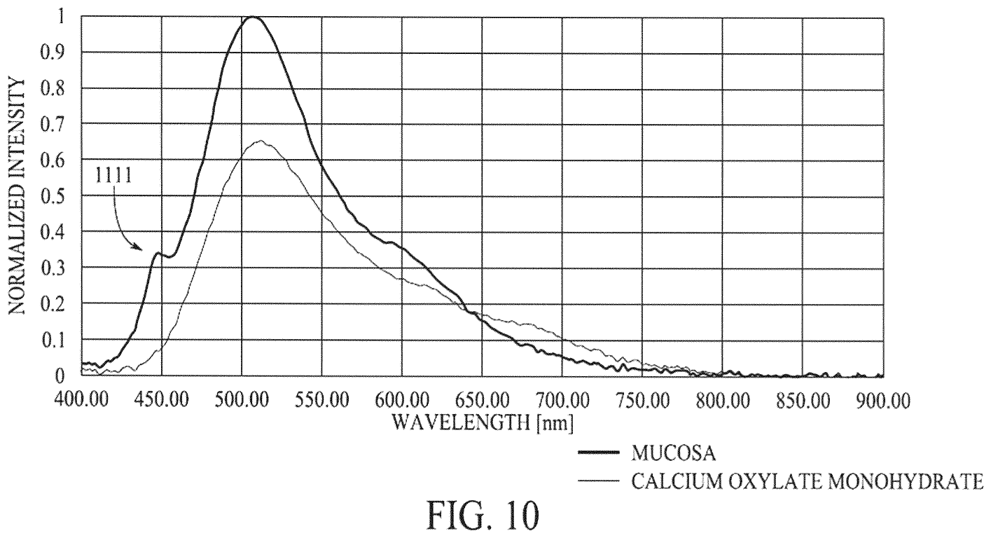

The choice of a suitable wavelength band(s) depends on the condition treated. For lithotripsy applications, this choice is dictated by the fluorescence spectra of stones and soft tissue. FIG. 10 shows the fluorescence response (x-axis=wavelength, y-axis=normalized intensity) of two different types of the biological material: calcium oxalate monohydrate (COM) and human soft tissue (mucosa) using an excitation wavelength of 365 nm. Of note is the distinctive feature 1111 around 450 nm of the mucosa spectrum. This feature can be used to further enhance differentiation between stone and soft tissue.

In accordance with a further non-limiting example, the dynamic characteristics of the fluorescent signal can be measured by the system 400. This can be achieved through either pulsing or modulating the excitation source 437 and performing detection of the fluorescent signal (response) in either the time or the frequency domain to measure the fluorescence lifetime. From these measurements, the fluorescent lifetimes can be determined. Identification of the target can be based on the contrast between characteristics of fluorophores found in the target areas (e.g., calculi) vs. the surrounding intact areas (e.g., soft tissues), e.g., the base difference in their respective fluorescence spectrum or lifetime. Fluorescence spectrum analysis can be performed by the spectrometer 448, or by measuring the signal in a predetermined spectral bandpass that is defined by spectral filters.

In yet another non-limiting example, a polarized excitation light source 437 can be used, and the polarization state of the fluorescence signal can be measured. In this case, the fiber optic instrument 445 is configured with a polarization-preserving channel to accurately transmit the polarization state to the sensors. Analysis of the polarization state of the fluorescent signal allows to indirectly evaluate fluorescence lifetimes and thus to reveal differences between the target and non-target areas.

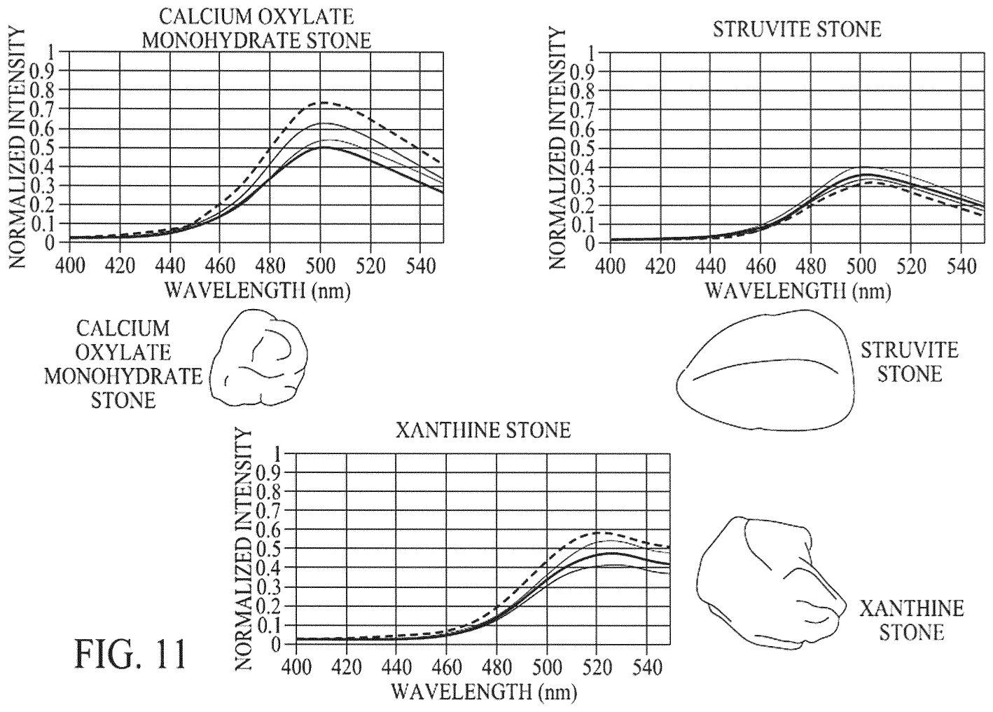

FIG. 11 shows graphs of the autofluorescence response (e.g., the fluorescent light spectrum) for three different types of calculi (e.g., calcium oxylate monohydrate, struvite and xanthine).

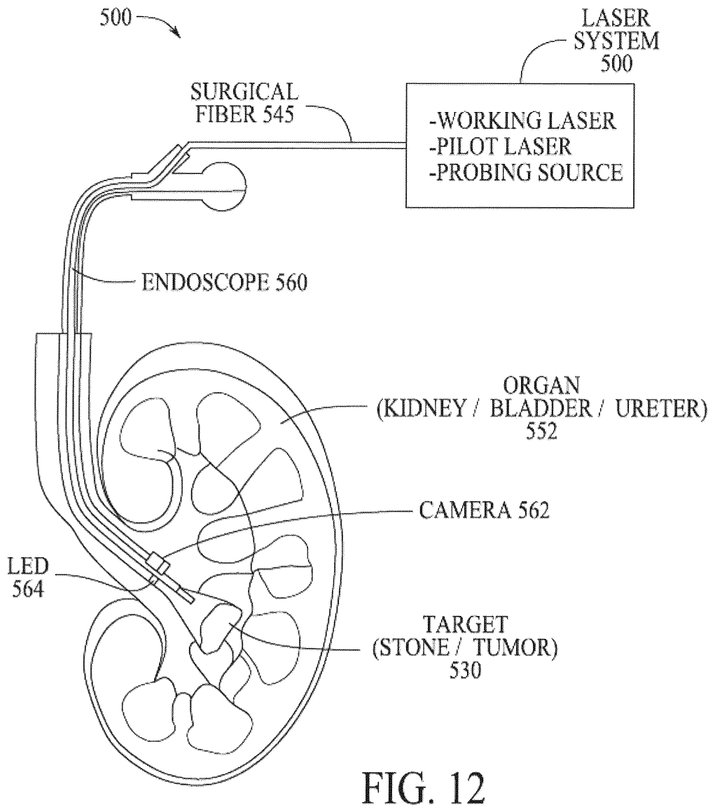

FIG. 12 shows a schematic of another example of a laser system 500 with a surgical fiber 545 and an endoscope 560. Laser system 500 can combine three types of light sources-a treatment laser, a pilot laser and a probing light source. Light is distributed or otherwise guided from the proximal end to the distal end of the surgical fiber 545. The surgical fiber 545 can be inserted into the endoscope 560 and may include a flexible component with the distal fiber tip. The end of the endoscope 560 can be directed into the patient's organ 552 (e.g., a urethra, a bladder, a ureter, a kidney, etc.). An illumination light source 564 (e.g., an LED light source) can be provided at the distal end of the shaft of the endoscope 560, as shown in FIG. 12. This light source 564 can illuminate the operation/manipulation field inside the organ (or other location inside the patient). The endoscope 560 can include a video camera (imaging sensor) 562 that can translate the real-time image of the operation/manipulation field onto an outside monitor/screen and optionally to an image processor. Using a camera and one or more analyzed signals from the smart sensor system, the doctor can guide the surgical fiber along the urethra, bladder, ureter, kidney channel to approach and find the target 530, such as a stone that needs to be fragmented, or soft tissue (e.g., tumor) that needs to be treated (vaporized, coagulated or excised).

The probing light source can be any one of a number of different light sources, including LED light sources with narrow or broad spectrums, and these light sources can have any number of different wavelength ranges, including wavelength ranges in the UV, visible, near IR range, etc. In some non-limiting examples, the probing light source can be a laser light source having any one of a number of different wavelengths, including those that match the peak absorption of a target chromophore, non-limiting examples of which include 400-450 nm, 500-600 nm, 940-1100 nm, 1150-1350 nm, 1400-1600 nm, and 1850-2200 nm. In some instances, these wavelength(s) may correspond to specific physiological features. For instance, 400-450 nm and 500-600 nm relate to hemoglobin absorption, which can be used to distinguish between tissue and stone materials because soft tissue contains hemoglobin and stone does not (e.g., tissues absorb these wavelengths more than calculi). In another example, 520-540 nm is related to a range of commonly used aiming beam wavelengths (for example, see aiming beam in optical adapter of FIG. 5) and the aiming beam can be used as a probing beam. In another example, 940-1000 nm, 1400-1600 nm and 1850-2200 nm relate to peaks of water absorption (tissue contains more water than stones), and 400-940 nm or 1150-1350 nm are related to the opposite case of water transmission. Therefore, these wavelengths can show different probing responses from stone vs. soft tissue, which can be used to distinguish between these two types of physiological materials.

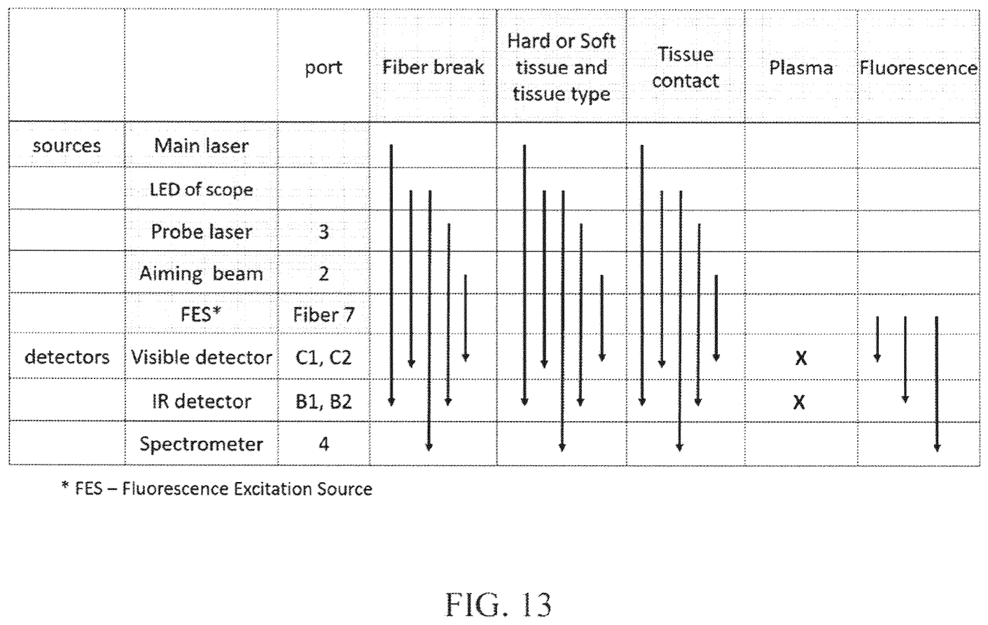

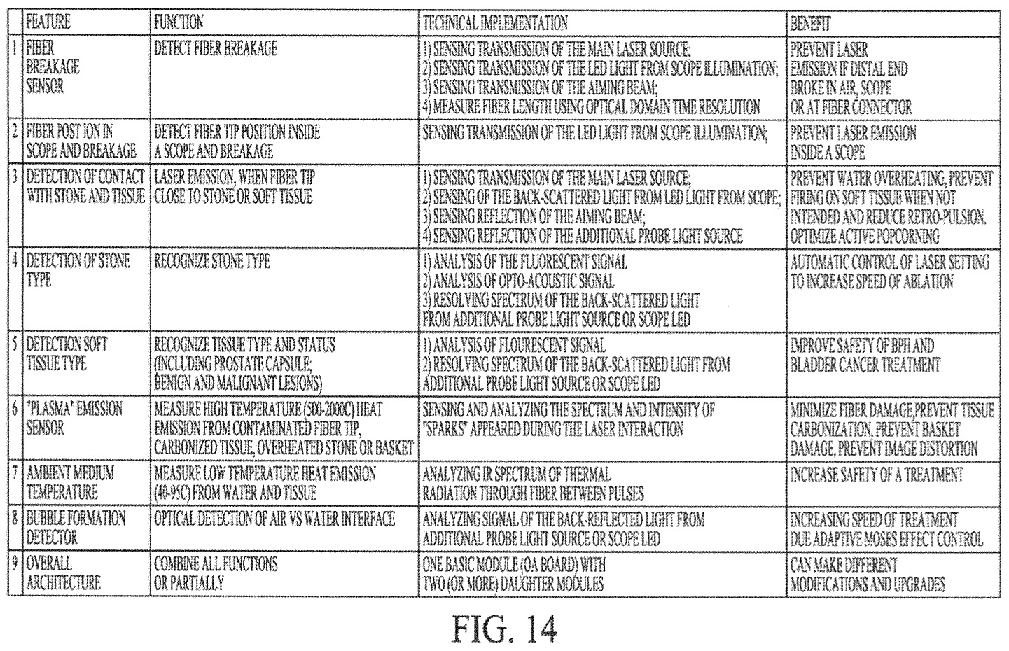

FIG. 13 shows a table of certain functionalities of the laser systems described herein (e.g., which can include determination of a treatment condition), while FIG. 14 shows a more specific table of the table of FIG. 13 (e.g., describing each functionality in more detail). Some of the other figures (and corresponding description) provide information about these functionalities and others. For example, FIG. 13 shows a non-limiting list of functionalities of an optical adapter of a laser system. Arrows show different combination of diagnostic light sources and detectors. As indicated in these figures (and others), elements of the optical adapter and related plurality of detectors, light sources, and probe signal data from the probe light source can be organized to perform the non-limiting list of “smart” functions listed herein to ensure more efficient, safer, and faster overall treatment procedures with improved clinical outcomes. The systems described herein can be used in whole or in part for multiple functions. For instance, different probe signal data and different back-reflected signals can provide information of particular conditions within a specific surgical environment.

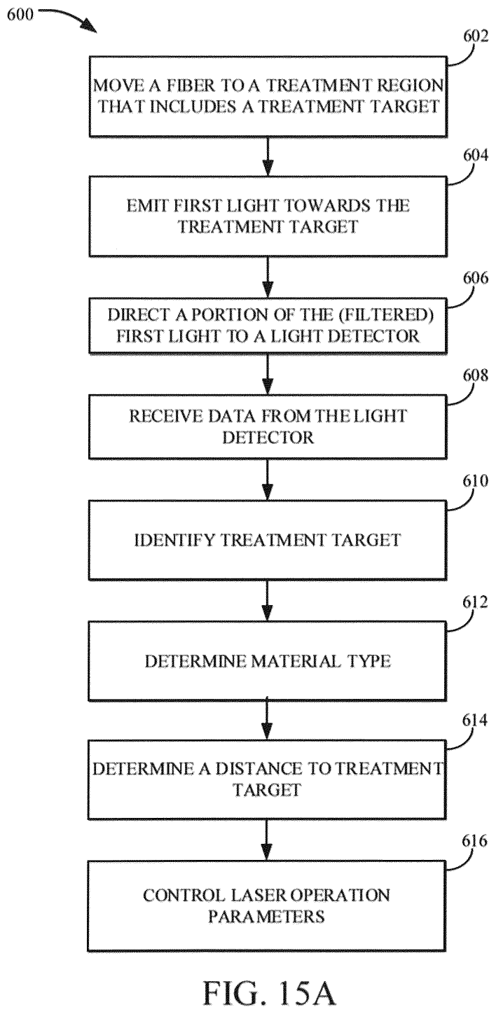

FIG. 15A shows a flowchart of a process 600 for determining that a treatment target is a target material or is tissue. As described above, a target material may be a stone or calculus, or other material. The stone or calculus may be located in a target area such as the bladder, ureter or the kidney, or located elsewhere in the renal or urinary system. Additionally or alternatively, the target material may not be a stone or calculus or may not be associated with the renal or urinary system, but may be a material located elsewhere in the body or in other systems in the body.

The process 600 can be implemented using any of the laser systems described herein (e.g., the laser system 100), and the process 600 can be implemented using one or more computing devices as appropriate (e.g., the computing device 130). Furthermore, as will be described below, the process may utilize a robotic surgical system or may utilize clinician control instead of robotic control.

At 602, the process 600 can include moving a fiber to a treatment region that includes a treatment target (e.g., a target material). In one non-limiting example, the fiber can be moved manually. As another non-limiting example, a computing device can cause a robotic surgical system to move the fiber to the treatment region, and can cause the fiber to be at a desired position relative to the treatment target. In some cases, this can include a computing device inserting the fiber into a tube of a medical scope, and inserting the medical scope (e.g., with the fiber positioned therein), into a patient. In one example, the insertion, whether manual or robotic, may be via a urethra of the patient. In other cases, a clinician or a computing device can cause the shaft of the medical scope to be inserted into the patient within the treatment region, and subsequently, can cause the fiber to be inserted into a working channel of the shaft of the medical scope until a distal end of the fiber is inserted through the shaft of the medical scope. While this discussion has been described with reference to a computing device, such is just one non-limiting example. The process 600 can include a practitioner or clinician controlling the system and the medical scope, including positioning the scope and/or fiber in the patient and moving the fiber until the distal end of the fiber reaches the treatment region (e.g., at the predetermined distance from the treatment target).

At 604, the process 600 can include a computing device (or the practitioner) causing a light source to emit a first light toward the treatment region. For example, the light source can emit first light into a proximal end of the fiber, which can propagate through the fiber and can be emitted out the distal end of the fiber into the treatment region. In some cases, the light source can be positioned within or proximate to the treatment region (e.g., being coupled to the medical scope). In some cases, the first light can be a broad continuous spectrum of light, or the first light can include one or more wavelengths within a range between substantially 400 nm to substantially 750 nm. In some cases, the first light can be pulsed (e.g., having multiple pulses, each of which has a pulse width). In some cases, the first light can be white light (e.g., the first light source being configured to emit the white light, such as a white LED). In some configurations, the first light can be coherent light (e.g., with the light source being a laser source). In some cases, the first light can be non-treatment light (e.g., the first light not being configured to elicit a therapeutic response when the first light is directed at a target, which can include ablation, coagulation, etc.). In some cases, the first light can have an average power of less than 100 mW. In this way, the first light does not undesirably interact with the treatment target, which can disrupt receiving of a portion of the first light, thereby disrupting the identification of the treatment target.

At 606, the process 600 can include directing a portion of the first light to a light detector. In some cases, the portion of the first light can be transmitted back into the distal end of the fiber, can propagate through the fiber, and can be emitted out of the proximal end of the fiber and directed to a detector (e.g., via an optical adapter). In some cases, the portion of the first light can be passed through an optical filter before reaching a detector. Thus, for example, the process 600 can include filtering the portion of the first light by passing the portion of the first light though an optical filter that restricts the portion of the first light to wavelengths within a range (e.g., the visible light range). In some cases, the optical filter can be in optical communication with an optical filter, which can be positioned within a port of an optical adapter. In some configurations, a portion of the first light can be backscattered, back-reflected, or the like.

At 608, the process 600 can include a computing device receiving data from the light detector. For example, the data can correspond to the portion of the first light (e.g., having been filtered) interacting with the light detector. In some cases, the light detector can be a spectrometer. In some cases, the data can include an intensity for one or more wavelengths of the portion of the first light. For example, the one or more wavelengths can be within a range between substantially 350 nm to substantially 750 nm, a range between substantially 400 nm to substantially 700 nm, etc. In some cases, the data can include an optical back reflected spectrum. In some cases, a computing device can normalize the data, based on the light emission spectrum of the light source (e.g., because the amplitudes of the first light are not entirely uniform across all wavelengths of the first light). In some cases, a computing device can filter the data (e.g., using a low pass filter, a high pass filter, a band pass filter, a band stop filter), which can remove one or more intensity values (for one or more wavelengths within a wavelength range), can amplify intensity values, or the like.

At A 610, the process 600 can include a computing device determining that the treatment target is a tissue or a target material, based on the optical data. Optical data are signals from optical detectors (photosensors). Examples include photodiodes, 1D or 2D matrix of photo sensors (charge-coupled device (CCD) as an example). 1D matrix optical data are produced by spectrometer and 2D data are produced by imaging sensor. Optical data represent electrical current or voltage from optical detectors in analog or digital form that can be subsequently transmitted to a computing device.

A data profile or optical data profile or optical data temporal profile can refer to data or optical data as a function of time. The data profile can be a single optical data profile from a single detector or a matrix of optical data profiles from a 1D or 2D matrix of photosensors. As will also be described, a characteristic optical data profile or characteristic optical data temporal profile can refer to a stored/known data as a function of time, whereby the underlying material or material properties that produced the profile are known, such as a predefined calibrated or preset profile. To this end, a characteristic data profile can be used to identify a data profile in a target environment during a clinical procedure. That is, the data profile can be compared with the characteristic data profile during treatment to control the treatment laser, as will be described.

For example, a computing device can analyze an intensity of the light at a selected wavelength or wavelengths using the data, for example, by comparing an optical data profile to a characteristic data profile, or other criteria, and can determine that the treatment target is a tissue or a target material based on the comparison. As a more specific example, a computing device can analyze an intensity profile of the optical signal acquired by the detector and compare this detected optical profile to a characteristic optical data profile that serves as a criteria against which to determine that the treatment target is a target material.

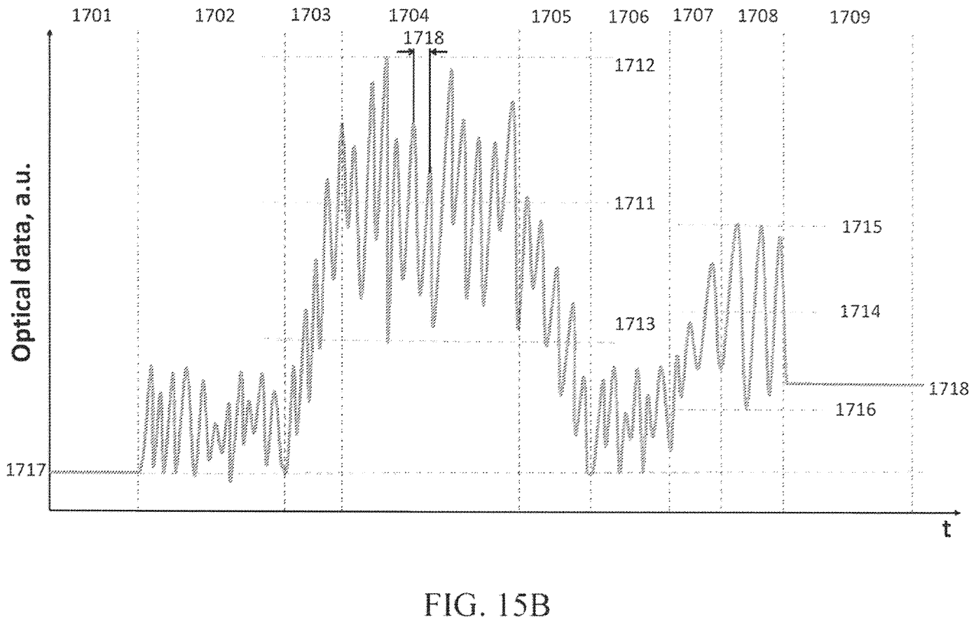

Furthermore, such can be used as characteristic criteria. For purposes of the present application, “characteristic criteria” can mean a previously-generated optical data profile associated with a specific analytical conclusion based upon pre-clinical or clinical collection studies stored in the computing device. Such characteristic criteria may be temporal in nature, such as will be described with respect to FIG. 15B which provides a signal profile for reflecting light indicating stone or tissue being targeted, or may be based upon absolute or relative units, such as the spectral profiles found in FIGS. 16, 17, and 18 identifying tissue and types of stones.

Referring to FIG. 15B, a graph is provided showing one example of an optical data profile for use in laser treatment, such as described above. Data or optical data collected from the detector, which is received back refection light of illumination sources of the surgical scope or probing light (target area) from the surgical environment (target area). The back reflection light intensity or power is continuously changing during treatment and has a different level and time behavior depending on the position of the distal end of the fiber and scope relevant to the target and non-target materials and laser operation. When the distal end of the fiber is far from the target, which can be 1 to several millimeters (time interval 1701 and 1702) the level of back reflection light 1717 is low and shows some scattering from the overall environment, including liquid and a wall of treatment organs. If a surgeon activates a laser on this fiber and scope position, the back refection signal will be oscillating due to the treatment laser induced bubbles forming and collapsing on the distal end of the surgical fiber and corresponding back refection scattered on these bubbles in interval 1702. When the fiber has been moved closer to a stone surface at 1703 (about 1-2 mm), the back reflected light intensity will increase toward a maximal level achieved when in contact with the stone at 1704. Notably, the oscillation amplitude and irregularity can also increase at the same time, due to additional back refection of light caused by the product of stone ablation. During 1705, the distal end of the fiber has lost contact with the stone. When losing contact with the stone, the back reflection signal decreases, as illustrated at 1705, toward a level similar to pre-contact with stone at interval 1706. If the surgeon moves the fiber towards soft tissue, such as the ureter wall, the back reflection signal will increase at interval 1707 to a maximal level reached when in contact with the soft tissue, while the amplitude of oscillation will increase at 1708. As will be described, upon determining such a data profile, the laser sources may be disabled, the laser power may be decreased, the energy or interval between pulses may be adjusted, or the like, either by the surgeon or the system. This continues as the back reflection signal drops to a level typical for back reflection from tissue.

During all this time, the control system performs real-time comparisons of this optical data profile with a characteristic optical data profile. Several criteria can be used for the comparison. For example, the signal level 1717 and 1718 can be compared with known, non-contact and contact levels for contact with a target or without contact with a target, with or without treatment laser operation. Furthermore, average levels 1711, 1714, maximal 1712 1712, 1716, minimal levels 1713, 1716, an interval between oscillations 1718, length of oscillation, and a statistic related to time intervals and amplitude of oscillation or the like may be evaluated. Thus, the characteristic or known optical data profile or key attributes thereof are processed against or compared to the optical data profiles collected in pre-clinical or clinical studies for different surgical treatment environments. The computing device (control system) performs a comparison in real-time of the optical data profile with the characteristic optical data profile using one or multiple criteria.

Additionally or alternatively, the computing device may determine an integrated intensity by determining the area under the optical backreflection spectrum within a wavelength range (e.g., from 540 nm to 590 nm), or by summing together each intensity value for each wavelength of the data within a wavelength range. Then, a computing device can determine that the treatment target is a target material based on the integrated intensity value being greater than a criteria value, or a computing device can determine that the treatment target is a tissue based on the integrated intensity value being less than the criteria value.

In some cases, including when the data includes an optical absorbance spectrum, a computing device can compare the optical backreflection spectrum to a first predetermined optical backreflection spectrum associated with a target material (e.g., an average of multiple optical backreflection spectrums each acquired from different calculi), and a second predetermined or characteristic optical backreflection spectrum associated with a tissue (e.g., an average of multiple optical backreflection spectrums each acquired from different tissues). Then, a computing device can determine which of the first or the second predetermined optical backreflection spectrums the optical backreflection spectrum matched closer with, and can corresponding determine that the treatment target is a target material (based on the optical backreflection spectrum being closer to the first predetermined optical backreflection spectrum) or can determine that the treatment target is a tissue (based on the optical backreflection spectrum being closer to the second predetermined optical backreflection spectrum). In some cases, this matching can include a computing device determining an amount of overlap between two respective optical backreflection spectrums.

At 612, the process 600 can include a computing device determining a type of target material (e.g. a uric stone, a calcium oxalate monohydrate stone, a cysteine stone, or the like.) for the treatment target based on the data, after for example, a computing device determined that the treatment target is a target material. In some cases, a computing device can follow a similar process at block 610 to determine the type of target material from a plurality of possible types of target material. For example, a computing device can compare a profile or an intensity value for a wavelength to a criteria value, and can determine the type of target material based on the comparison to a characteristic profile or intensity. As another example, a computing device can compare an integrated intensity value to a criteria, and can determine the type of target material based on the comparison. In some cases, a computing device can determine a type of target material, based on comparing an intensity profile from the data to a characteristic profile. Additionally or alternatively, the computing device can compare an amplitude of a wavelength(s) to one or more criteria values. For example, a computing device can determine that the target material is a uric stone, based on an amplitude of a selected wavelength of the data (or an integrated intensity value) being greater than a first criteria and a second criteria, with the second criteria being larger than the first criteria. As another example, a computing device can determine that the target material is a calcium oxalate monohydrate stone, based on the amplitude of the wavelength of the data (or the integrated intensity value) being between the first criteria and the second criteria. As yet another example, a computing device can determine that the target material is a cysteine stone, based on the amplitude of the wavelength of the data (or the integrated intensity value) being less than the first criteria and the second criteria.

At 614, the process 600 can include a computing device for determining a distance between the distal end of the fiber and the treatment target, based on the determined treatment target. For example, different treatment target types or features (e.g., size) can have a corresponding predetermined desired distance associated therewith (e.g., stored in a database). As a more specific example, a computing device can receive a predetermined distance that is associated with the determined treatment target or target feature, such as size, (e.g., in a database). For example, a computing device can receive a predetermined distance for the treatment target corresponding to a target material (and/or size of the target material), based on a computing device determining that the treatment target is a target material (and the type of the target material). Correspondingly, a computing device can receive a predetermined distance for the treatment target corresponding to a tissue, based on the computing device determining that the treatment target is a tissue. In some cases, this can be advantageous, in that predetermined distances can be optimized for specific treatment targets. For example, treatment targets that are tissue are desired to have a greater distance than calculi (e.g., because stones benefit more from laser light being focused more closely at the stone corresponding to better ablative performance, while tissues benefit more from a more disperse laser light corresponding to better coagulation). In addition, harder stone types can benefit from a smaller distance (e.g., more focused laser light being directed at a harder stone) as opposed to softer stone. Thus, for example, when the treatment target has been determined to be stone target material, the distance can be smaller than a predetermined distance for a tissue, and when the treatment target has been determined to be tissue, the distance can be greater than a predetermined distance for stone. Correspondingly, when the treatment target has been determined to be a target material having hard material property, the distance can be less than a predetermined distance for a target material having a softer material property than the hard material property.

At 616, the process 600 can include a computing device determining laser operation parameters for a treatment laser, based on the above analysis. In some cases, the laser operation parameters can include a pulse peak power, a pulse shape a pulse width of laser light emitted by a treatment laser, an interval between pulses, a frequency of the laser light, a power of the laser light (e.g., an average power), a total duration of laser light, and the like. In some cases, a computing device can receive one or more predetermined laser operation parameters for the treatment target corresponding to a target material (and a type of the target material), based on a computing device determining that the treatment target is a target material (and the type of the target material). In other cases, a computing device can receive one or more predetermined laser operation parameters for the treatment target corresponding to a tissue, based on the computing device determining that the treatment target is a tissue. In some non-limiting examples, having predetermined laser operation parameters can be advantageous in that the predetermined laser operation parameters can be tailored to a specific treatment target and type thereof. For example, tissues benefit from CW power operation and calculi benefits from pulse operation with high peak power (e.g., because higher amounts of laser light directed to calculi can be advantages to fracture the target material). Thus, one or more predetermined laser operation parameters for tissue can be lower than one or more predetermined laser operation parameters for calculi (and vice versa). Similarly, one or more predetermined laser operation parameters for a first type of calculi can be higher than one or more predetermined laser operation parameters for a second type of calculi (e.g., with the first type of calculi being harder than the second type of calculi).

In some non-limiting examples, block 616 can include a computing device notifying a practitioner based on the results from one or more determinations. For example, a computing device can present on a display of a laser system or endoscopic image the results of the determination from the block 610, which can include presenting on a display that the treatment target is a target material (and a type thereof), or that the treatment target is tissue. In addition, a computing device can present on a display the determined distance (e.g., a predetermined distance) associated with the treatment target, can present on the display the determined laser operation parameters associated with the treatment target, etc.

In some non-limiting examples, the treatment target has already been predetermined to be tissue, or target material (and a type thereof). In this case, for example, the process 600 can be used to determine that the current treatment target (e.g., in front of the distal end of the fiber) matches with the predetermined treatment target. In this case, a computing device can determine that the current treatment target (e.g., determined at the block 610, 612) corresponds or does not correspond with the predetermined treatment target. If a computing device determines that the current treatment target matches with the predetermined treatment target, then a computing device can control operation of the treatment laser (e.g., including enabling firing of the treatment laser, causing the treatment laser to emit laser light, increasing one or more laser operation parameters for the treatment laser to operate according to, etc.). However, if a computing device determines that the current treatment target does not match with the predetermined treatment target (e.g., that the predetermined treatment target is stone and the current treatment target is tissue), then the computing device can control operation of the treatment laser (e.g., including disabling firing of the treatment laser, stopping the treatment laser from emitting laser light, changing one or more laser operation parameters, etc.). In addition, if the computing device determines that the current treatment target does not match with the predetermined treatment target, then the computing device can alert a practitioner by, for example, presenting an alert on the display, flashing or sound. In this way, during a laser procedure, a computing device can, in real time, adjust operation of the treatment laser if the current treatment target is not the actual predetermined treatment target, which can prevent undesirable firing of the laser, increase treatment efficiency and safety, etc.

In some non-limiting examples, the treatment target can be identified to be a stone target material versus tissue, because stone can reflect, scatter, etc., a higher amount of light than does tissue-especially within particular wavelength ranges (e.g., a wavelength ranges of 410 nm to 460 nm and 550 nm to 590 nm) in which the tissue absorbs light within the wavelength range (e.g., due to hemoglobin absorbing the light). For example, FIG. 16 is a graph showing examples of the optical backreflected spectrums of back reflected light (reflected and scattered from a surface of stone or tissue, back scattered from bulk stone or tissue, etc.) of a scope LED illumination from different types of stone and kidney tissue as shown in FIG. 12. Back reflected light propagated through the surgical fiber was directed into another fiber which was connected to a spectrometer (Thorlabs Inc., CCS100/M 350-700 nm) using the configuration shown in FIG. 6 (E.g., the light being directed through lenses and by beam splitter(s)). The surgical fiber had a core diameter of 0.2 mm and extended from the ureteroscope tip by 3 mm. The distance between the surgical fiber tip and the surface of the stone or tissue was about 1 mm. Analyzing the spectra shown in FIG. 16, it was discovered that there are substantial differences between the spectra of different stone types (e.g., COM, Uric Acid, Cysteine) and between the spectra of stone and soft tissues. Different stone types have different levels of reflection in all wavelength ranges. Soft tissue has a specific local minimum in the range 540-590 nm, which can be used for soft tissue identification.

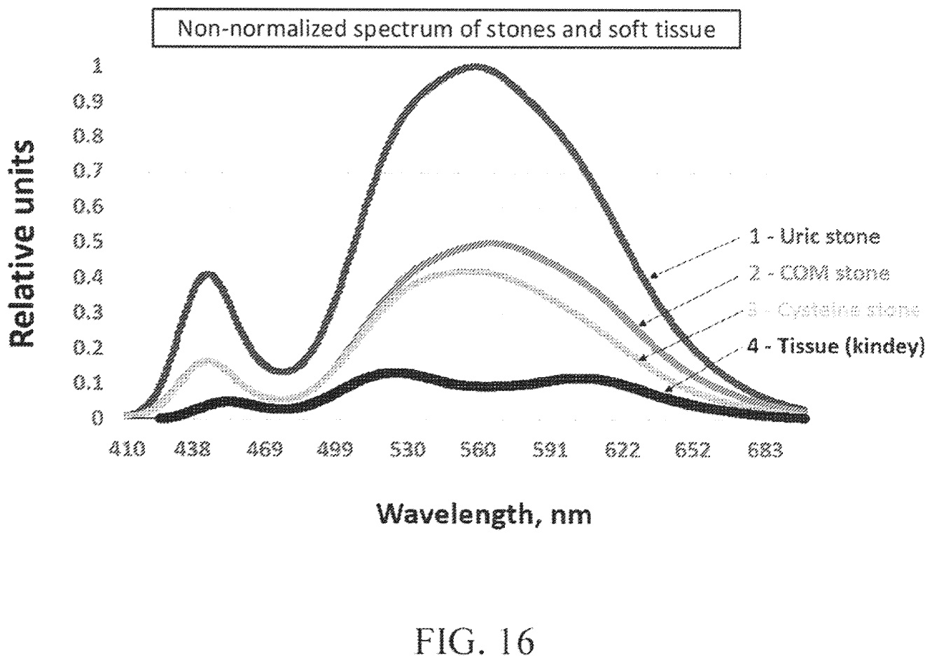

FIG. 17 is a graph showing examples of the same spectra of back reflected/scattered LED light from stones and from soft tissue but normalized to the original LED spectrum. These spectra can be used for identification of different types of stones and soft tissues. Various stone types and their differentiation against soft tissue can be identified by spectrum analysis (e.g., through differentiating or integrating the spectral curves in all regions or in the most sensitive spectral ranges). This information can be used for identification of stone or stone type and soft tissue before applying laser energy.

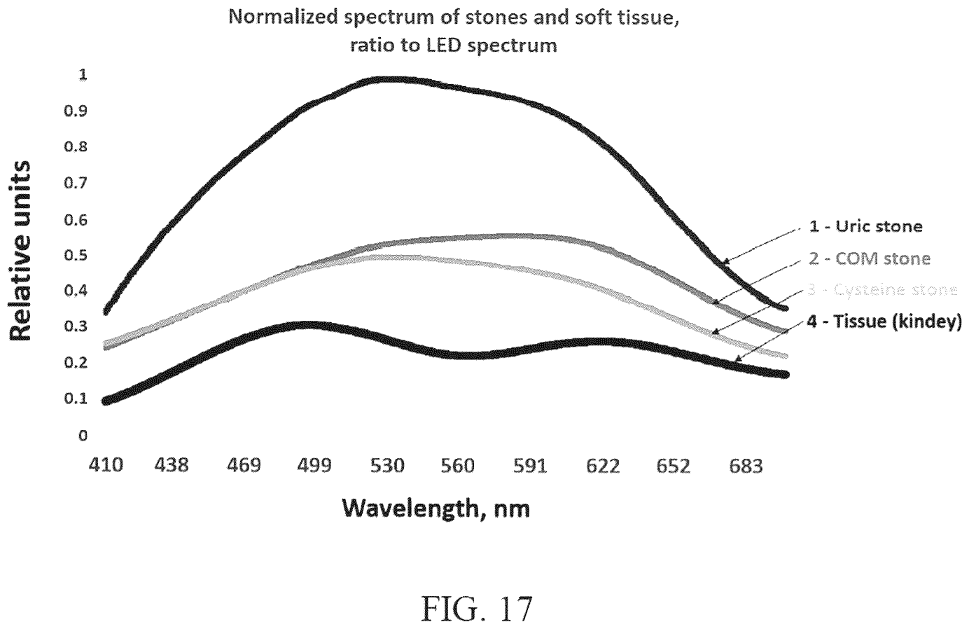

FIG. 18, which is a table, shows an integral of back reflected/scattered spectra of endoscopic LED signals from stones and soft tissue in different spectral ranges. The table shows examples of the integral back reflected LED signals from stones and soft tissue in different wavelength ranges: whole range of 410-700 nm, blue ranges of 410-460 nm, green-yellow range 510-620 nm, and preferable narrow ranges of 410-430 nm, 550-590 nm. Tissue type (hard tissue or soft tissue) and stone type (e.g., an exact stone type) can be identified by integral signals in wavelength ranges mentioned above. For example, during laser treatment of a kidney stone in a ureter, a surgeon should keep the distal end of the surgical fiber in contact with stone. But if a surgeon accidentally loses contact with the stone and touches the ureteral wall and continues firing, the wall can be perforated with unacceptable side effect and may require open surgical intervention. These experiments showed a surprisingly high difference in back reflection signal from stone and soft tissue (2 to 4 times depending on stone type). When the back refection signal decreases more than 1.2-1.7 times during treatment, the laser system can send an audible and/or visual warning signal to a surgeon to stop firing or the laser system can stop lasing automatically.

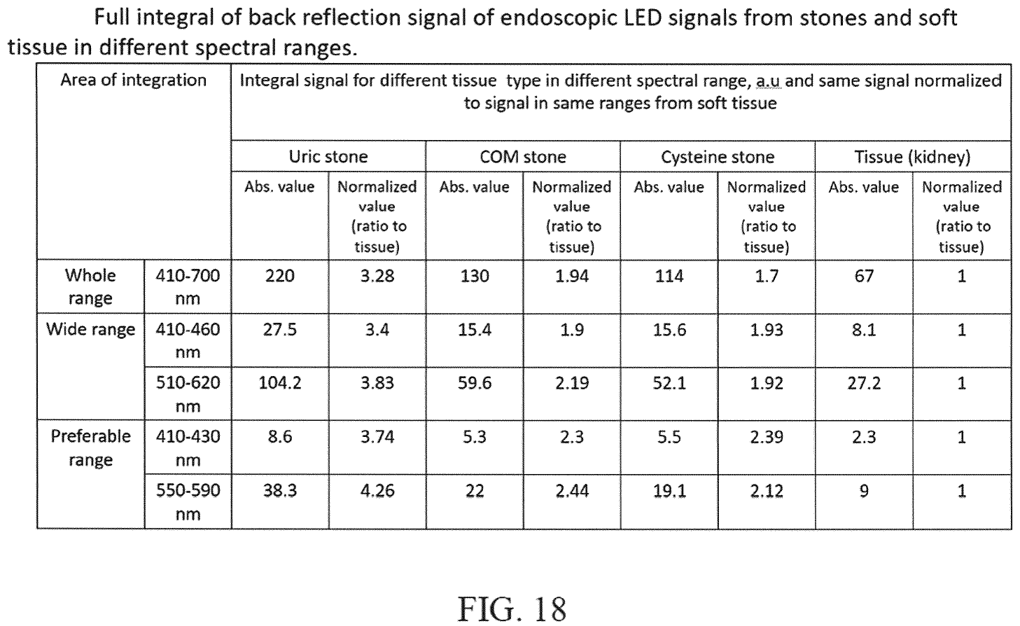

FIG. 19 is a graph showing examples of the spectra of back reflected/scattered LED light from stone divided (normalized) by the spectra of the back reflection LED light from soft tissue. Variations in this ratio can be used for identifying stone vs soft tissue.

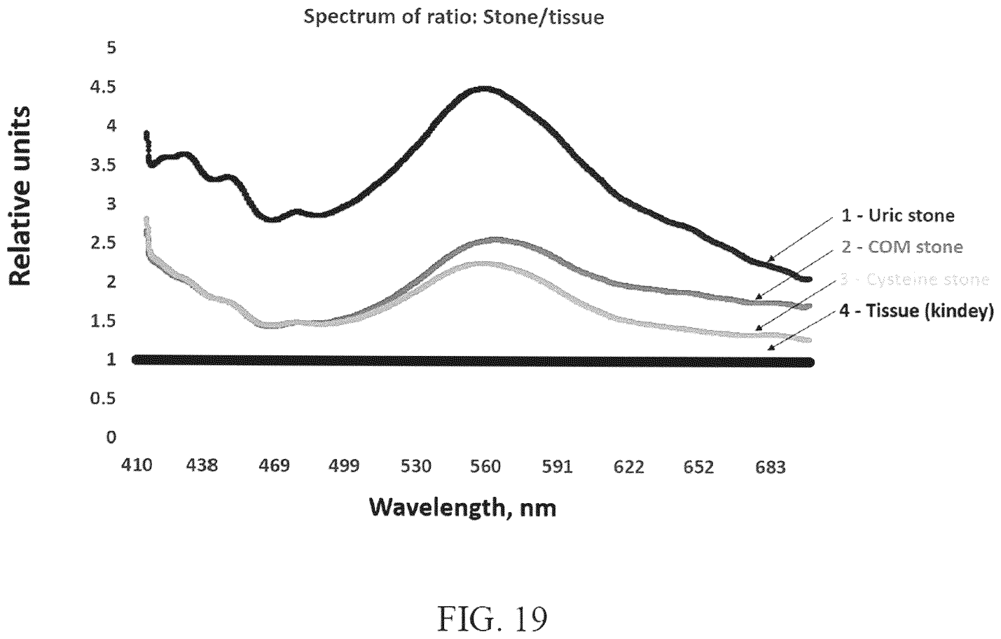

FIG. 20 shows a flowchart of a process 650 for determining that a particular treatment target is a target material and not an undesired target, such as healthy tissue. The process 650 can be implemented using any of the laser systems described herein (e.g., the laser system 100), and the process 650 can be implemented using one or more computing devices as appropriate (e.g., the computing device).

In accordance with another non-limiting example, an acoustic signal induced in the medium by absorption of a pulse of laser energy can be used by a laser system to identify the type of treatment target (e.g., tissue vs. target material). In addition, laser induced the acoustic signal can be used to detect and monitor the formation of air bubbles in the medium. The acoustic signatures of various tissue types and stones can differ from one another due to differences in the chemical compositions and in the geometrical structures and as results differences acoustic signal during laser light absorption and ablation, and this information can be used by the laser system to distinguish between these types of materials. One or both of the intensity and the spectral characteristics of the opto-acoustic signal can be used to identity a treatment target. The acquired (received) acoustic signal can be used to inform the operator, and can be used by a computing device to control pulsing of the treatment laser.

The acoustic signal can be acquired or otherwise received by an acoustic receiver such as a microphone placed at one of the following locations: on distal tip of shaft of the scope, as an independent tool inside the working channel of the scope, or an acoustic receiver that is attached to the skin of a patient in close proximity to the treatment area. Either the laser used for treatment or a specially introduced probe laser light source can be used to induce the acoustic signal. In accordance with one non-limiting example, a range of pulse widths used for this purpose can be between 1 ns and 20 ms, and a range of acoustic frequencies received can be between 10 Hz and 50,000 Hz.

At 652, the process 650 can include a computing device moving a fiber to a treatment region that includes a treatment target, which can be similar to the block 602 of the process 600.

At 654, the process 650 can include a computing device causing a treatment laser to emit laser light towards the treatment region (e.g., and at the treatment target). This can include the laser light being emitted into the proximal end of the fiber, propagating along the fiber, and being emitted out of the distal end of the fiber into the treatment region. In some cases, the treatment laser can emit the laser light according to one or more laser operation parameters. In some cases, the laser light can be pulsed (e.g., the laser light includes one or more pulses separated from each other). While the block 654 described laser light being directed towards the treatment target, in other configurations, a computing device can cause a light source to emit light towards the treatment target (e.g., which can be different than the laser light from the treatment laser, such as having less power than the laser light from the treatment laser).

At 656, the process 650 can include generating acoustic waves based on the interaction between the laser light (or the light from the light source) and the treatment target. At 658, the process 650 can include a computing device receiving acoustic data from an acoustic transducer, corresponding to the acoustic waves interacting with the acoustic transducer. In some cases, a computing device can filter the acoustic data (e.g., by passing the acoustic data through a filter to reduce acoustic intensity values, amplify acoustic intensity values, and the like).

At 660, the process 650 can include a computing device determining that the treatment target is a tissue or a target material, based on the acoustic data. The block 660 can be similar to the block 610, except that rather than the data being analyzed, the acoustic data can be analyzed. Thus, the acoustic data can be analyzed in a similar manner as the analysis of the data with regard to the block 610. For example, a computing device can compare an intensity value of a frequency of the acoustic data to an intensity value criteria, and can determine that the treatment target is a tissue or a target material based on the comparison. As a more specific example, a computing device can compare an intensity value of a frequency of the acoustic data to an intensity value criteria, and can determine that the treatment target is a target material based on the intensity value being greater than the intensity value criteria or can determine that the treatment target is tissue based on the intensity value being less than the intensity value criteria.

In some cases, a more robust approach to determining that the treatment target is tissue or target material can include a computing device determining an integrated intensity value from the acoustic data. For example, a computing device can determine the integrated intensity by determining the area under the acoustic spectrum within a frequency range (e.g., from 10 Hz to 10 kHz), or by summing together each intensity value for each frequency of the acoustic data within a frequency range. Then, a computing device can determine that the treatment target is a target material based on the integrated intensity value being greater than a criteria, or a computing device can determine that the treatment target is a tissue based on the integrated intensity value being less than the criteria.

In some cases, including when the acoustic data includes an acoustic spectrum, a computing device can compare the acoustic spectrum to a first predetermined acoustic spectrum associated with a target material (e.g., an average of multiple acoustic spectrums each acquired from different calculi), and a second predetermined acoustic spectrum associated with a tissue (e.g., an average of multiple acoustic spectrums each acquired from different tissues). Then, a computing device can determine which of the first or the second predetermined acoustic spectrums the optical absorbance spectrum matched closer with, and can corresponding determine that the treatment target is a target material (based on the acoustic spectrum being closer to the first predetermined acoustic spectrum) or can determine that the treatment target is a tissue (based on the acoustic spectrum being closer to the second predetermined acoustic spectrum). In some cases, this matching can include a computing device determining an amount of overlap between two respective acoustic spectrums.

At 662, the process 650 can include a computing device determining a type of target material based on the acoustic data, after, for example, the treatment target has been determined to be a target material. This can be similar to the block 612 of the process 600, except that acoustic data is used by a computing device instead of the data (e.g., but with the same determination steps of the block 612).

In some non-limiting examples, a computing device can determine a distance for the distal end of the fiber and the treatment target, based on the determined treatment target (and type thereof) in a similar manner as the block 614 of the process 600.

At 664, the process 650 can include a computing device determining a state of the treatment target, based on the acoustic data. For example, a computing device can determine that a treatment target (e.g., that has been determined to be a target material) has been ablated, carbonized, coagulated, based on an amplitude for one or more frequency of the acoustic data exceeding (e.g., being greater than) a criteria associated with the respective state (e.g., ablated, carbonized, coagulated, etc.).

At 666, the process 650 can include a computing device determining a presence of a bubble (e.g., at the distal end of the fiber, at the treatment target, etc.). In some cases, an acoustic spectrum in the presence of a bubble is different than an acoustic spectrum in the absence of a bubble. In some cases, determining a presence (or absence) of a bubble can follow a similar process as the blocks 610, 612, 660, 662. For example, a computing device can compare each amplitude for one or more frequencies of the acoustic data to a criteria (or multiple criteria), and based on each amplitude exceeding the criteria can determine that the presence (or lack of) a bubble. Bubble presence can be identified based on amplitude of acoustic signal with frequency corresponding to the frequency of laser pulses inducing the bubble. For example, an increase in the amplitude of the acoustic signal above a certain threshold (e.g., by a factor of 10 higher than background signal) can indicate onset of bubble formation. The frequency range is preferably between 10 Hz and 10 kHz.

At 668, the process 650 can include a computing device determining laser operation parameters for a treatment laser (e.g., to emit laser light at a treatment target), based on the determined treatment target, the presence (or lack thereof) of a bubble, etc. This can be similar to the block 616 of the process 650. In addition, the block 666 can include notifying a practitioner based on the results of the one or more determinations, which can also be similar to the block 616 of the process 650.

In some non-limiting examples, similarly to the process 600, the treatment target can be predetermined to be tissue, or target material (and a type thereof) for the process 650. In this case, a computing device can determine that a current treatment target (e.g., in front of the distal end of the fiber), which can be determined at the block 610, 612, matches (or does not match) with the predetermined treatment target. The results of this can dictate different adjustments, notifications, alerts, etc., in a similar manner to the process 600.

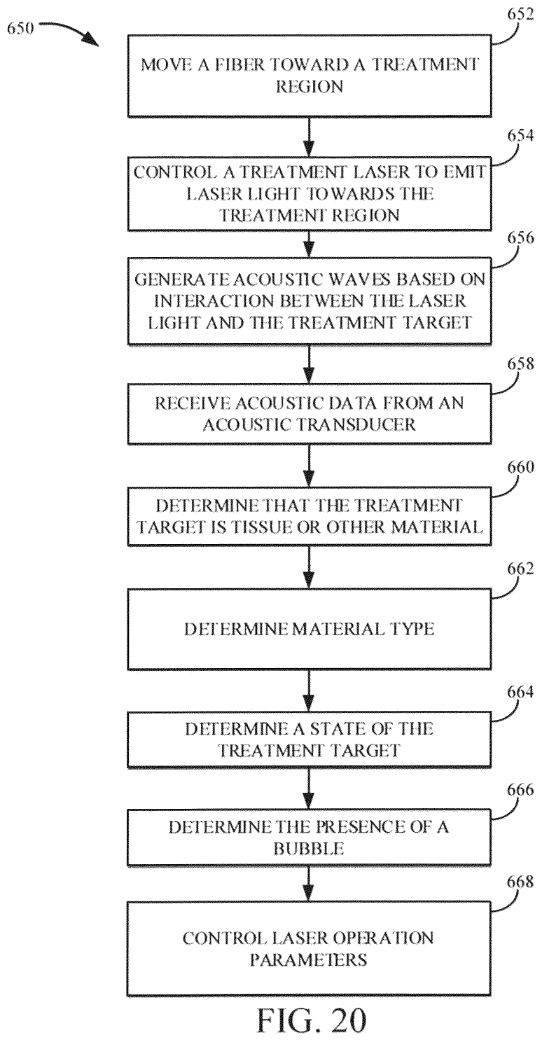

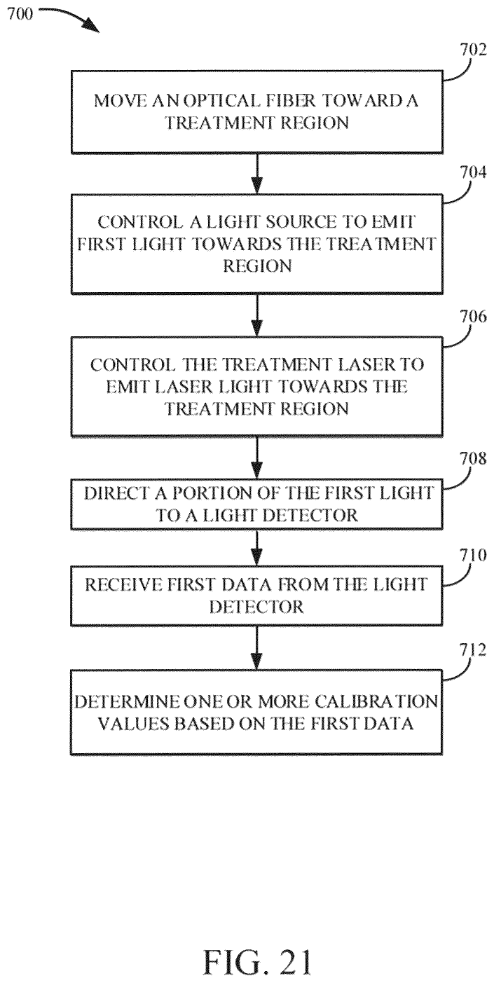

FIGS. 21 and 22 collectively show a flowchart of a process 700 for determining a distance between a distal end of a fiber and a treatment target. The process 700 can be implemented using any of the laser systems described herein (e.g., the laser system 100), and the process 700 can be implemented using one or more computing devices as appropriate (e.g., the computing device 130).

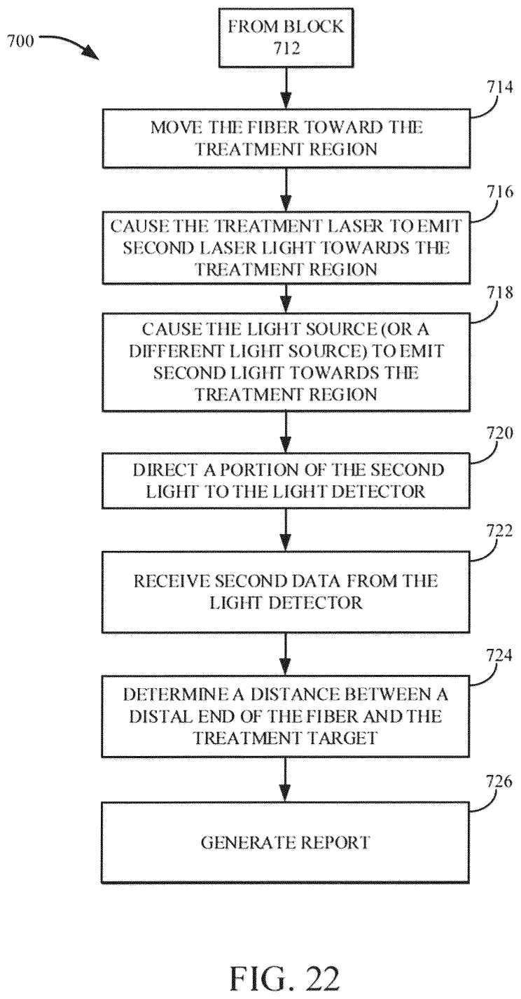

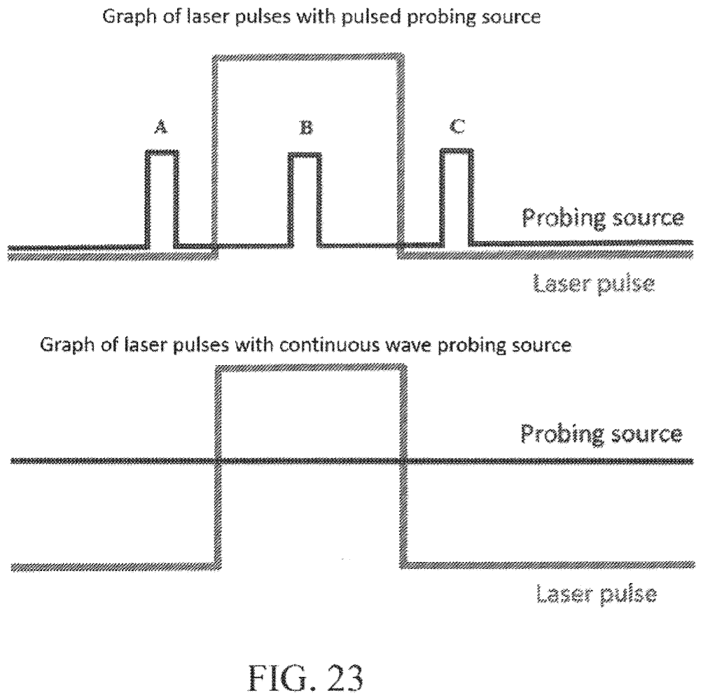

At 702, the process 700 can include a computing device moving a fiber to a treatment region that includes a treatment target, which can be similar to block 602 of process 600. At 704, process 700 can include a computing device causing a light source to emit a first light toward the treatment region, according to a calibration procedure, which can be similar to block 604 of process 600. At 706, the process 700 can include a computing device causing a treatment laser to emit laser light toward the treatment region, according to the calibration procedure, which can be similar to the block 654 of the process 650. In some cases, the laser light according to the calibration can have a laser pulse, and the first light can have one or more pulses (e.g., three pulses). In some cases, a first pulse of the first light can be emitted before the laser pulse (e.g., the first light being emitted before the leading edge of the laser pulse), a second pulse of the first light can be emitted during emission of the laser pulse (e.g., the second pulse being situated between a rising edge of the laser pulse and a falling edge of the laser pulse), and a third pulse of the first light can be emitted after emission of the laser light (e.g., after the trailing edge of the laser pulse). An example of this configuration is shown in the upper region of FIG. 23, in which the first light is the probing source (light), and the first, second, and third pulses of the first light correspond to the pulse A, the pulse B, and the pulse C, respectively.

In some non-limiting examples, the first light can be emitted continuously before, during, and after the emission of the laser pulse. For example, the first light can include a first pulse which can be emitted before, during, and after the emission of the laser pulse. An example of this configuration is shown in the lower region of FIG. 23, in which the first light is the probing source (light) that is emitted before, during, and after the laser pulse.

Referring back to FIG. 21, at the block 708, the process 700 can include directing a portion of the first light to a light detector, which can be similar to block 606 of process 600. At block 710, process 700 can include a computing device receiving first data from the light detector, which can correspond to the portion of the first light (e.g., having been filtered) interacting with the light detector. Block 710 can be similar to block 608 of process 600. In some configurations, the portion of the first light, which is directed to the light detector to generate the first data, can be directed back into the distal end of the fiber and emitted out the proximal end of the fiber to the light detector. The portion of the first light can correspond to one or more sections of the first light, with a first section being emitted before the laser pulse, with a second section being emitted during the laser pulse, and with a third section being emitted after the laser pulse.

At 712, process 700 can include a computing device determining one or more calibration values, based on the first data (e.g., which can be filtered). In some cases, the data can include one or more first intensity values corresponding to the first light being emitted before the laser pulse (e.g., the first section of the portion of the first light), one or more second intensity values corresponding to the first light being emitted during the emission of the laser pulse (e.g., the second section of the portion of the first light), and one or more third intensity values corresponding to the first light being emitted after the emission of the laser pulse (e.g., the third section of the portion of the first light). In some non-limiting examples, a computing device can determine a first calibration value from the one or more first intensity values (e.g., by averaging them together), can determine a second calibration value from the one or more second intensity values (e.g., by averaging them together), and can determine a third calibration value from the one or more third intensity values (e.g., by averaging them). Each of the calibration values can be utilized to more accurately determine the distance. For example, the one or more first, second, and third intensity values each correspond to different conditions of the treatment region. Namely, the one or more first intensity values can correspond to the treatment region being free of a bubble (e.g., at the distal end of the fiber), the one or more second intensity values can correspond to the treatment region including a bubble (e.g., at the distal end of the fiber), and the one or more third intensity values can correspond to the treatment region including a vapor channel through the bubble. In this way, depending on the relationship between the emission of subsequent light relative in time to the emission of subsequent laser light, the distance determination can be more accurate.

At 714, process 700 can include a computing device moving the fiber to the treatment region. In some cases, this can include a computing device moving a distal end of the fiber to a predetermined distance (e.g., using process 600, 650) relative to the treatment target. Block 714 can be similar to block 702.

At 716, process 700 can include a computing device causing the treatment laser to emit second laser light towards the treatment region, which can be similar to the block 654 of the process 650. In some cases, block 716 can be omitted if, for example, the treatment laser is to only emit laser light after determining the distance.

At 718, process 700 can include a computing device causing the light source (or a different light source) to emit a second light toward the treatment region, which can be similar to block 704. In some cases, using the same light source can be advantageous in that the calibration procedure can be tailored to the particular light source.

At 720, process 700 can include directing a portion of the second light (e.g., which can be filtered) to the light detector (or a different light detector), which can be similar to block 708. At 722, process 700 can include a computing device receiving (and filtering) second data from the light detector (or the different light detector), which can be similar to block 710.

At 724, process 700 can include a computing device determining a distance between a distal end of the fiber and the treatment target based on the second data (and based on one or more of the calibration values). In some cases, a computing device can compare an intensity value from the second data to a curve that relates intensity values and distances (of the distal end of the fiber to the treatment target). In some cases, the curve can be associated with the type of treatment target (e.g., target material (and corresponding type) or tissue). In some cases, a computing device can calibrate the second data by applying one or more (e.g., a combination) of the first, second, or third calibration values to each intensity value of the second data, depending on when the second data was acquired relative to the second laser light (if applicable). For example, if the second laser light was not emitted at all, or if the second laser light was emitted after, before, not during, etc., the second light, then the first calibration value can be applied to the second data (e.g., to each intensity value of the second data). In some cases, calibrating the second data can include subtracting each intensity value of the second data from the first calibration value, and dividing by the first calibration value. In other words, calibrating the second data can include determining a relative change between each intensity value of the second data and the first calibration value.

In some configurations, determining the distance between the distal end of the fiber and the treatment target at block 726 can include repeating blocks 718-722 to cause the light source (or another light source) to emit third light, which can be directed to the light detector (or a different light detector), to generate third data that can be received by a computing device. In this case, a computing device can determine a change between an intensity value (e.g., calibrated according to one or more of the calibration values) of the third data and an intensity value (e.g., calibrated according to one or more of the calibration values) of the second data (e.g., by subtracting the data). Then, a computing device can compare the change in intensity values to a curve that relates the derivative of the intensity values and the distances (of the distal end of the fiber to the treatment target). In some cases, using the change in intensity values can be a more robust way to determine the distance.

At 726, the process can include a computing device notifying a practitioner, based on the results from the one or more determinations, and displaying the results. Block 726 can be similar to block 616 of process 600. In some cases, this can include a computing device presenting on a display the distance.

In some non-limiting examples, when a predetermined distance has already been determined or received by a computing device, the distance determined at block 724 can be a current distance. In this case, a computing device can determine the difference between the current distance and the predetermined distance, and present on a display the difference (or otherwise notify a practitioner of the difference). In some cases, if a computing device determines that the current distance exceeds a predetermined distance, then a computing device can control operation of the treatment laser (e.g., including enabling firing of the treatment laser, causing the treatment laser to emit laser light, increasing one or more laser operation parameters for the treatment laser to operate according to, etc.). However, if a computing device determines that the current distance does not exceed the predetermined distance, then the computing device can control operation of the treatment laser (e.g., including disabling firing of the treatment laser, stopping the treatment laser from emitting laser light, decreasing one or more laser operation parameters, etc.). In addition, if the computing device determines that the current distance exceeds the predetermined distance, then the computing device can alert a practitioner by, for example, presenting an alert on the display. In this way, during a laser procedure, a computing device can, in real time, adjust operation of the treatment laser if the current distance deviates from the predetermined distance, which can prevent undesirable firing of the laser, can increase treatment efficiency, etc. For example, sometimes during laser treatment the target material moves (e.g., known as retropulsion) and in this case because the distance between the distal end of the fiber and the treatment target can be determined, tissue is not undesirably treated with laser light (e.g., the computing device can cause the treatment laser to stop firing). As another example, during “popcorning” after the target material fractures into pieces and is suspended or floats in urine, the treatment laser is not always in the line of site with each of these particles. So, when a particle is close enough to the distal end of the fiber, the computing device can cause the treatment laser to fire, thereby creating an automatic firing procedure as particles are brought close enough to the distal end of the fiber-even when images from a medical scope are obscured.

In some non-limiting examples, process 700 can be described with reference to a specific implementation of the laser system. For example, at the beginning of a lithotripsy procedure, an endoscope can be manipulated such that the distal end of the fiber enters or is otherwise inserted into the channel of the kidney (or ureter). The distal end of the fiber is still virgin and the response signal of the probing light source as detected by the sensors (e.g., photodiode) is original and initially related only to Fresnel reflection on the distal end. A calibration procedure can be commenced at this time since there is no risk of tissue damage and there is no stone present. As such, the doctor can initiate the calibration procedure, e.g., by pressing a calibration pedal (button). Once initiated, a laser emits radiation one or several times with certain predetermined parameters (pulse power, pulse width, and frequency). Water overheats, which leads to bubble formation on the fiber tip side. Simultaneously, the computing device synchronizes the calibration pulse with the three (or more) original baseline (reference) pulsed probe source signals into memory: at the moment right before the pulse (moment A), during the pulse (moment B) and right after the pulse (moment C), as shown in FIG. 23. Basically, these disables power to the treatment laser so as not to cause tissue injury. The doctor can thus continue to find the stone and correct the position of the fiber tip. However, if the target is indeed tissue, the system allows the laser to turn on when K1>0.2 (but not more than 0.4) and the gap is about 200 microns or less. Only when K1 is over 0.4 and up to 1.4 or even more, should the computing device, in some cases, determine that it is indeed stone material situated in front of the fiber tip microns, the tip is in full contact with the target and the laser can be turned on. In automatic mode during this lithotripsy operation, the laser will be prevented from firing on soft tissue if the doctor touches the tissue accidentally during stone treatment, since a stone is always close or attached to the ureter or kidney. In other non-limiting examples, during calibration mode the laser can fire with a lower energy and power if the stone is not detected and automatically switch to a higher energy and power if a stone is detected. This minimizes the retropulsion effect and overheating of the liquid in the treatment area to prevent soft tissue damage.

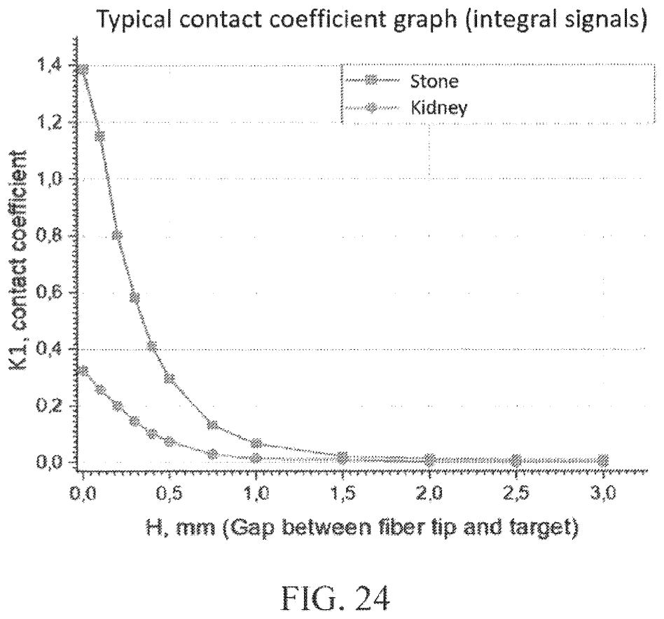

In some non-limiting examples, other characteristics of the measured signal can be advantageously used for more precise identification of the target and the distance to the target. As an example, FIG. 25 is a graph showing the relationship between the derivative of the contact coefficient and the gap between the fiber tip and the target, which can serve as a more sensitive measure of the target type and distance to its surface.

Once it is determined that the treatment laser can be enabled, the doctor can proceed with stone treatment. In some instances, a pedal may be used by the doctor for activating the treatment laser. Laser pulses with certain parameters (power, width, frequency etc.) start firing, which initiates several processes. First, if some kind of gap exists between the fiber tip and the target, then the laser pulses will cause local overheating and vaporize the water (existing in the gap) first. A bubble forms and starts to grow. The front side of the bubble reaches the stone surface to create a vapor channel and the “Moses effect” occurs. From this moment on, laser pulses impact not only the water but also the stone and thus become more “effective.” Further stone surface ablation leads to surface fragmentation and local crater formation and subsequent crater growth. Small stone particles created as a product of the laser ablation separate from the stone surface and track in all directions, including the fiber tip direction, and stone dusting occurs. Retropulsion can also occur, which leads to the stone moving and distancing itself from the fiber tip. If the pulse frequency is low enough, then after one pulse and before the next subsequent pulse, the bubble begins to collapse and a gap between the end of the fiber and the stone fills with water. After the bubble collapses, there is a stone with a crater in it at a distance from the front of the fiber tip. When the next pulse begins, this set of processes starts over again. During the time that the laser is turned on and during stone treatment, the response (probing signals detected by sensors) of the probing source changes dramatically due to backscattering from the product of ablation. This is due to several things, one being the changing nature of the amount of backscattering. Another is the change in the Fresnel reflection from the fiber tip according to the changing environment around the tip (e.g., “air” or “water” environment, smooth stone surface or stone surface with craters, distance to the stone, small particle tracking, lack of small particle tracking, absorption of laser light by a high temperature product of ablation, contaminated fiber distal end and heated area of fiber distal tip, etc.). Prior to the pulse, the level of the probing source response signal should be minimal (Fresnel reflection in water is less than on the air). During the laser pulse, the level of the probing source response signal can be at its maximum (Fresnel reflection on air is maximal, and backscattering of a porous crater and of tracking microparticles is maximal). In the time period right after the pulse, the probing source response signal should be something in between these minimum and maximum levels.

A computing device can receive all of the response signals related to the probing source, and can analyze these signals and control the laser system based on this analyses, e.g., determine whether the stone is still in the ablation area and not distanced to another area. If the stone is not in the treatment area, the control system automatically stops operation of the laser. To do this, the control system may need to implement additional “contact coefficient” parameters K2 and K3 that are similar to K1. In some instances, K2 is a parameter related to the moment B—during or right before the end of the pulse, where K2=(B1−B)/B), and K3 is related to the moment C-right after the pulse, where K3=(C1−C)/C). For example, B is a reference signal in “air” (bubble) that is measured during the calibration procedure and B1 is the current incoming signal during or right before the end of the pulse. Further, C is a reference signal and boundary condition between the A and B cases when the initial bubble is smaller or has even already collapsed and is measured during the calibration procedure, and C1 is the current incoming signal right after the pulse. These additional contact coefficient parameters can be determined during calibration, and like K1, K2 and K3 should also be evaluated in advance for each laser system design. The calculation of K1, K2, and K3 parameters during the calibration process and the use of these parameters in combination with real time results can provide the best probability of stone contact detection during laser pulses.

Different kidney and ureter stones (calculi) such as calcium oxylate monohydrate (COM stone), uric acid, cysteine, and others have different microstructures, different chemical compositions, (feasibly) different typical shapes and sizes, consist of different amounts of water inside, amongst other distinctions. Therefore these different types of calculi most likely have different backscattered and/or back-reflected probing source signals and thus have different contact coefficient parameters (K1, K2 and K3) when the distal end of the fiber is in front of the stone of some kind.

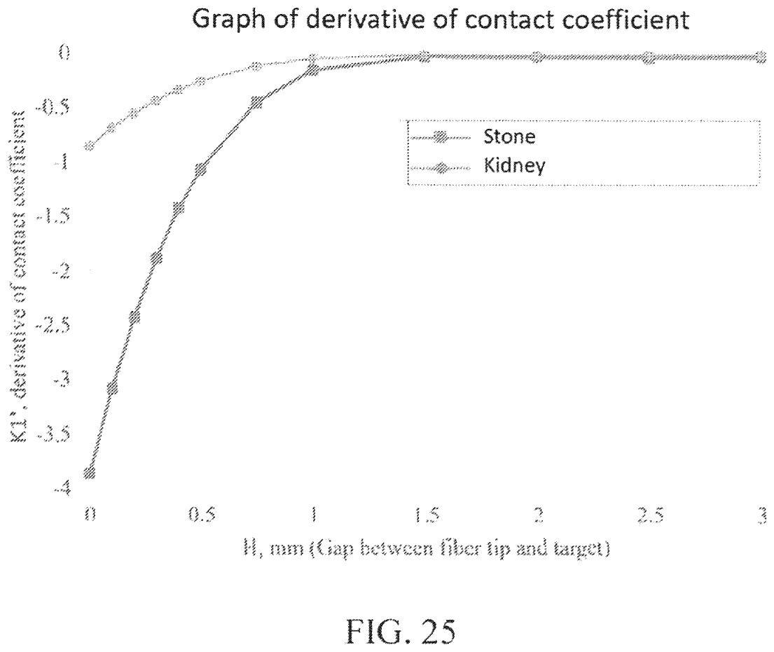

FIG. 26 is a graph showing the relationship between the contact coefficient K1 for different types of stone and soft tissue where the fiber tip is in contact with the target (the gap between the fiber tip and the target is 100 microns or less) using a probing source wavelength of 1550 nm in different 15 points of stone or tissue. According to one example, soft tissues (chicken breast, pork kidney and beef heart) 5-0.8, uric acid stone K1=0.6-1.8. However, it is to be appreciated that K1 can depend on various other parameters such as the probing source wavelength, the fiber diameter, the fiber distal end angle to stone surface, the condition of the fiber distal end, the distance between the fiber distal end to the stone surface (as described above), the current laser system design, and many other factors. Still, if these parameters are considered equal, the determined K1 coefficients will relate to different types of stone as is shown in FIG. 26. This can be used in real time to distinguish between different types of stone with a high probability. If the doctor is able to have such assistance during surgery with the described stone type detector, he or she (and/or the control system) will be able to select (choose) the best laser parameters (such as pulse power, width, frequency and other) in order to fragment the determined type of calculi with the highest possible efficiency. In some instances, this parameter can be predetermined and pre-set into the smart laser system (e.g., the control system) for purposes of proposing (suggesting) or otherwise conveying to the doctor the currently determined stone type that is in front of the fiber tip during surgery. This stone type detector is able to work when the laser is working. Furthermore, the stone type detector also works even when the stone is not homogeneous but chemically consists of two or more types of calculi, and/or has a changing fragmentation regime (mode) according to the current state (part) of stone in real time. This ability leads to preventing water overheating and shortens the time it takes to perform the surgery procedure. Changing or otherwise modifying a laser treatment parameter (e.g., laser operating parameter) according to the stone composition (based on feedback) can also be done automatically by the control system.

During operation, the fiber tip is subject to burn and degrade, which increases scattering from the fiber tip itself and eventually changes the response signals of the probing source regardless of changes induced by the stone backscattering during the treatment. This increases the probability of making a mistake in sensing contact with the target. In this instance, after a certain time period the doctor should repeat the calibration procedure. An alternative approach is to replace or cleave the damaged surgical fiber and perform a calibration procedure.

A computing device controlled laser system can therefore be used to receive and analyze response signals corresponding to the probing source in real time before, during, and after laser pulses for purposes of distinguishing between contact with stone vs. soft tissue. In addition, image data from the imaging sensor of scope can provide information regarding contact detection with stone and with soft tissue, and results from image processing can determine the stone and soft tissue type and condition. The system can also assist in controlling the laser for purposes of helping the doctor make more informed decisions, increase the efficiency of the stone treatment, shorten the procedure time, and to avoid injuries caused by improper positioning of the fiber tip during laser pulses where the camera's view is limited by stone dust.

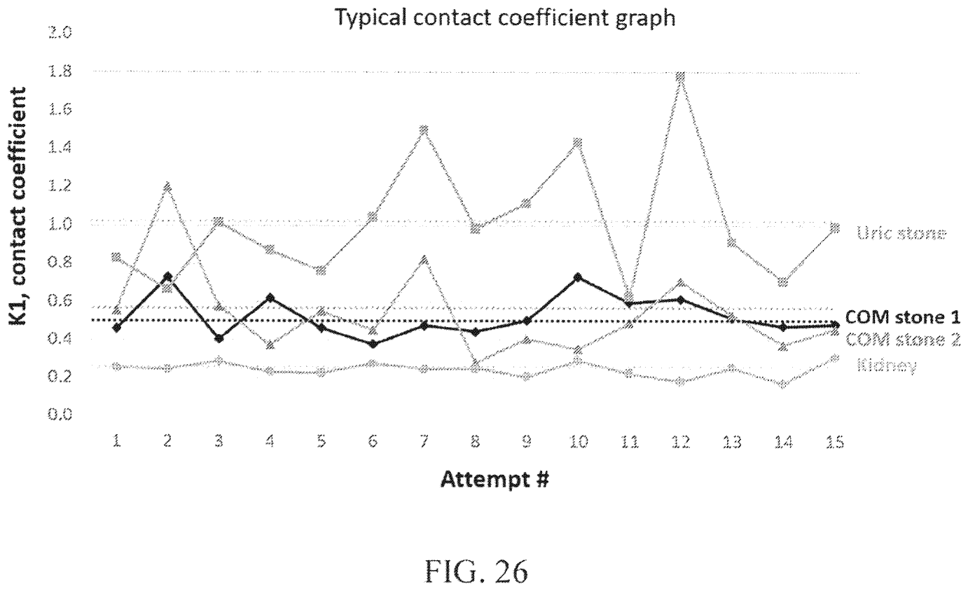

FIG. 27 shows a flowchart of a process 750 for determining a presence of “plasma”, which is visible light flashing, within a treatment region. The process 750 can be implemented using any of the laser systems described herein (e.g., the laser system 100), and the process 750 can be implemented using one or more computing devices as appropriate (e.g., the computing device 130).

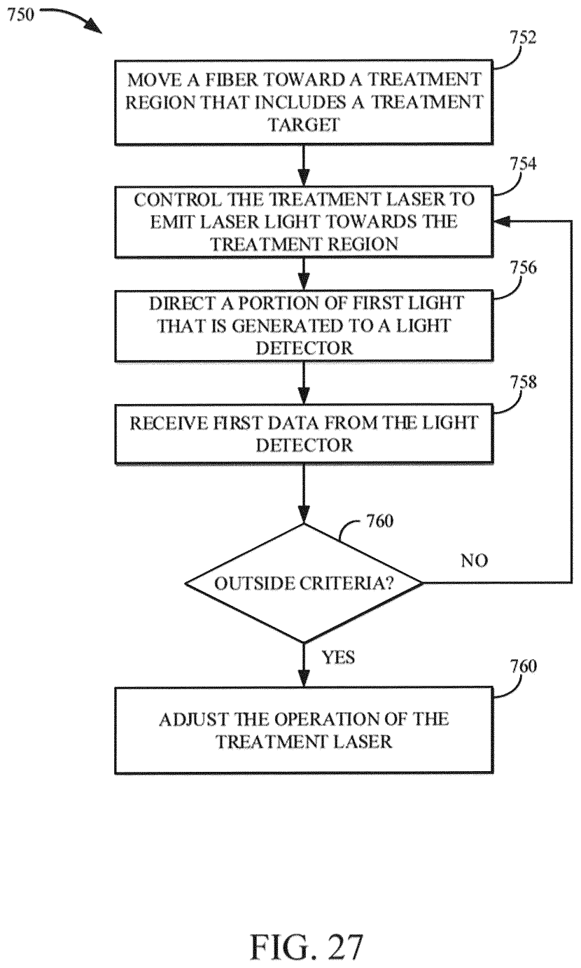

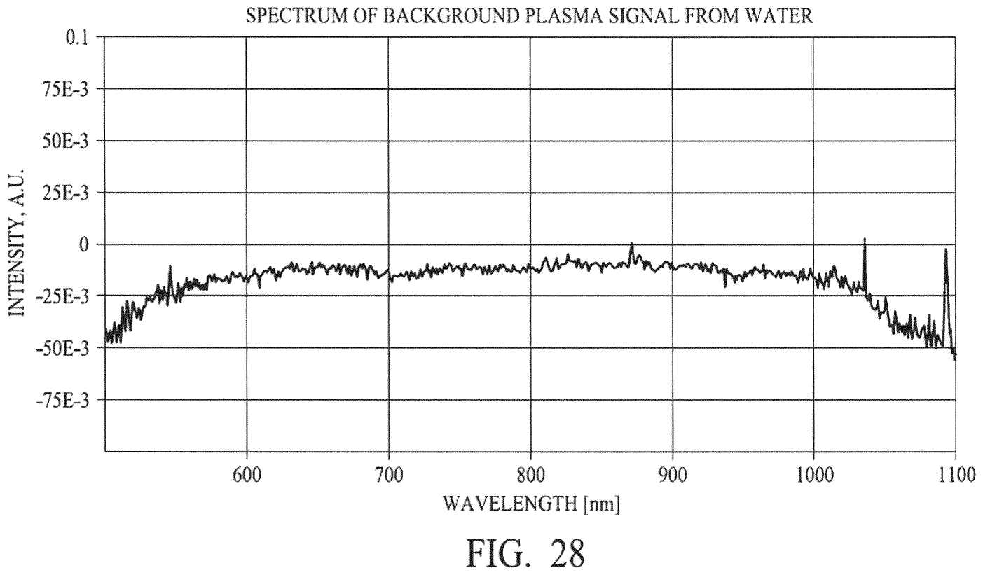

In some cases, when an object treated by a laser starts to emit electromagnetic radiation in the visible wavelength range that is referred to as “flashing” or “plasma” radiation. The physical origins of this phenomenon vary, and can range from thermal (heat) emission to different kinds of luminescence. As a practical matter, the phenomenon may impact image quality on the scope camera and negatively affect the operator's ability to target a desired area of tissue/stone. FIGS. 28 and 29 show two graphs that exemplify such a spectral response (x axis=wavelength, y-axis=intensity) for water (background) and a COM stone, respectively. In particular, FIG. 28 shows a spectrum of a background plasma signal (e.g., the first light generated) from the interaction of laser light and water, while FIG. 29 shows a spectrum of a plasma signal from the interaction between the laser light and a stone.

This type of emission can be associated with overheated carbonized tissue or carbonized proteins in stone. Another mechanism can be an overheated distal end of the fiber tip that is contaminated by a product of tissue or stone ablation that attaches to the fiber's distal end. The thermal temperature of this carbonized tissue, stone, or contaminated fiber can reach 1300-2400° C., and can be a cause of damage to the distal end of the fiber. If temperature of the fiber distal end exceeds 800° C., the fiber starts to absorb laser energy and becomes not-transmitting for the laser energy. As a result, the efficiency of stone or tissue ablation decreases. The disclosed smart laser system is configured to detect this phenomenon before it starts by performing at least one of the following: a) warn the operator to modify the treatment technique, b) pause laser treatment to abort development of the phenomenon, c) temporarily decrease the laser power and/or pulse energy and/or repetition rate, and/or d) irradiate fiber, tissue, and/or stone with a high peak power or high pulse energy to clean the carbonized tissue or ablate the product of ablation attached to the distal end of the fiber. Intensity and spectrum of heat radiation can be used to determine fiber tip temperature in real time and allow smart laser system to prevent distal tip temperature to exceed predetermine level in the range 500-800C and increase ablation efficiency and prevent fiber tip degradation.

This detection is implemented by performing optical monitoring on the laser-induced emission either during or immediately after each laser pulse. One non-limiting example of a spectral range where the signal is monitored is between 300 nm and 1900 nm, and 2100-2600 nm and preferably the range is between 400 nm and 900 nm or 400 to 1900 nm. The thermal emission signal can be directed into the treatment fiber and extracted at the proximal end through a dichroic beam splitter or a similar device. The thermal emission has a spectrum and intensity that is higher than other broad spectrum emission such as fluorescence or the illumination light of the scope, and can be distinguished by the light intensity level. Back reflection signals from the probe or laser beam can be selected using narrow bandpass filters upstream from a photodetector sensor. The control system (and/or operator) looks for an electromagnetic radiation signal (e.g., a feedback signal) having an intensity that meets and/or exceeds a predetermined criteria. This signal can be distinguished from any other feedback signals since measurements are received during a certain time window when two conditions are met: (1) the treatment laser or laser pulse is on, and (2) all other probing signals are off.

Once the onset of “flashing” is detected, the operator can be warned to modify the treatment technique to prevent further development of the phenomenon. For example, when the stones are treated, the phenomenon can be prevented (or at least significantly attenuated) by using the so-called “dancing” technique, where the fiber tip is rapidly moved along the surface of the treated stone. Other technique modifications are also possible.

In accordance with another aspect, at the onset of “flashing” the laser emission can be paused for a short period of time (in the range 0.05 to 1 s), which can also be used to prevent this phenomenon.

At 752, the process 750 can include a computing device moving a fiber to a treatment region that includes a treatment target, which can be similar to the block 602 of the process 600. At 754, the process 750 can include a computing device causing a treatment laser to emit laser light towards the treatment region (e.g., and at the treatment target), which can be similar to the block 654. At 756, the process 756 can include directing a portion of first light that is generated to a light detector. For example, the first light can be indicative of plasma generation within the treatment region, on the fiber, etc. For example, the first light can be generated from the interaction between the laser light and the treatment target, from the interaction between the laser light and a carbonized material, etc. In some cases, a portion of the first light can be transmitted back from the distal end of the fiber and out the proximal end of the fiber to the light detector. In some cases, the portion of the first light can be filtered prior to being directed to the light detector.

At 758, the process 750 can include a computing device receiving (and filtering) data from the light detector corresponding to the portion of the first light interacting with the light detector.

At 760, the process 750 can include a computing device determining whether or not the data exceeds a criteria. If at 760, the computing device determines that the data exceeded (e.g., being greater than) a criteria (e.g., an intensity value of the data exceeded a criteria), then the process 750 can proceed to the block 762. However, if the computing device determines that the data did not exceed the criteria, the process 750 can proceed back to the block 754 in which the treatment laser can continue emitting the laser light (or other laser light).

At 762, the process 750 can include a computing device adjusting the operation of the treatment laser, and notifying a practitioner, each of which can be based on the data, an in particular, that the data exceeded the criteria. In some cases, adjusting the operation of the treatment laser can include adjusting (e.g., lowering) one or more laser operation parameters for the treatment laser, pausing emission of the laser light, stopping emission of the laser light, lowering the power of the laser light, decreasing the pulse width of the laser light. In some cases, this can include increasing the laser operation parameters (e.g., the power of the laser light) to clean the distal end of the fiber. In this case, a computing device can determine that a distal end of the fiber (or other portion thereof) is dirty based on the data, can correspondingly proceed to the block 760 to adjust the operation of the treatment laser, which can include increasing the power of the laser light to clean the laser.

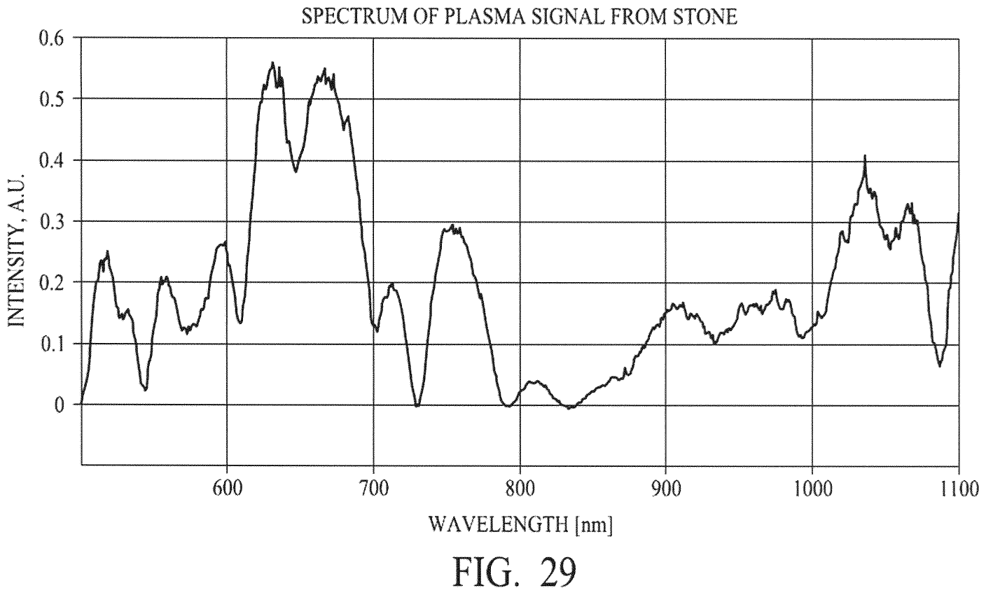

FIG. 30 shows a flowchart of a process 800 for determining a temperature for a component of a laser system, or a treatment region. The process 800 can be implemented using any of the laser systems described herein (e.g., the laser system 100), and the process 800 can be implemented using one or more computing devices as appropriate (e.g., the computing device 130).

Residual absorption of laser radiation in the liquid medium as well as heat transfer from the target stones/tissues may cause the temperature to increase in the corresponding organs (e.g., kidney, ureter, or bladder). Damage can occur if the temperature inside these organs exceeds ˜42° C. Therefore, it is important to monitor any increase in temperature and to prevent excessive increases in temperature.

According to at least one non-limiting example, the laser systems herein can be configured with temperature monitoring capabilities to prevent such overheating. Temperature monitoring can be implemented through detection and interpretation (analysis) of a thermal emission signal (also referred to herein as thermal radiation) transmitted through the same fiber that is used for the laser treatment. In some non-limiting examples, the wavelength range of the detected signal can be between 1500 nm and 10000 nm, but in some instances this will be limited by the transmission characteristics of the fiber used. According to another non-limiting examples, the wavelength range is between 300 nm and 2700 nm. In instances where silica fiber is used, the upper wavelength boundary is limited at about 2300 nm.

Calibration of the temperature meter (sensor) can be performed at the factory. If the temperature exceeds a pre-defined criteria, the control system can halt laser emission and warn the operator.

At 802, the process 800 can include a computing device moving a fiber to a treatment region that includes a treatment target, which can be similar to the block 602 of the process 600. At 804, the process 800 can include a computing device causing a treatment laser to emit laser light towards the treatment region (e.g., and at the treatment target), which can be similar to the block 654.

At 806, the process 800 can include a computing device receiving temperature data from a temperature sensor. In some cases, the temperature data can include one or more temperature values. In some cases, the temperature sensor can be in thermal communication with the fiber, with the treatment region (e.g., located within the treatment region), etc. In some cases, the temperature data can be derived from data (e.g., the data being derived from black-body radiation, from a portion of light from a heating light source that reflects off a target, including one or more structures of the treatment region, etc.).

At 808, the process can include a computing device determining whether or not the temperature data exceeds (e.g., is greater than) a temperature criteria. If at 808, the computing device determines that the temperature data exceeded the temperature criteria (e.g., that a temperature value of the temperature data exceeded the temperature value), then the process 800 can proceed to the block 810. If at 808, however, if the computing device determines that the temperature data did not exceed the temperature criteria, then the process 800 can proceed back to the block 804 to continue emitting the laser light (or to emit different laser light). In some non-limiting examples, the temperature criteria can be substantially 42° C.

At 810, the process 800 can include a computing device adjusting the operation of the treatment laser, notifying a practitioner, displaying the results, etc., based on the temperature data exceeding the criteria temperature, which can be similar to the block 760 of the process 750. For example, this can include a computing device pausing emission of the laser light for a period of time (e.g., a period of time within the range of substantially 0.05 seconds to substantially 1 second).

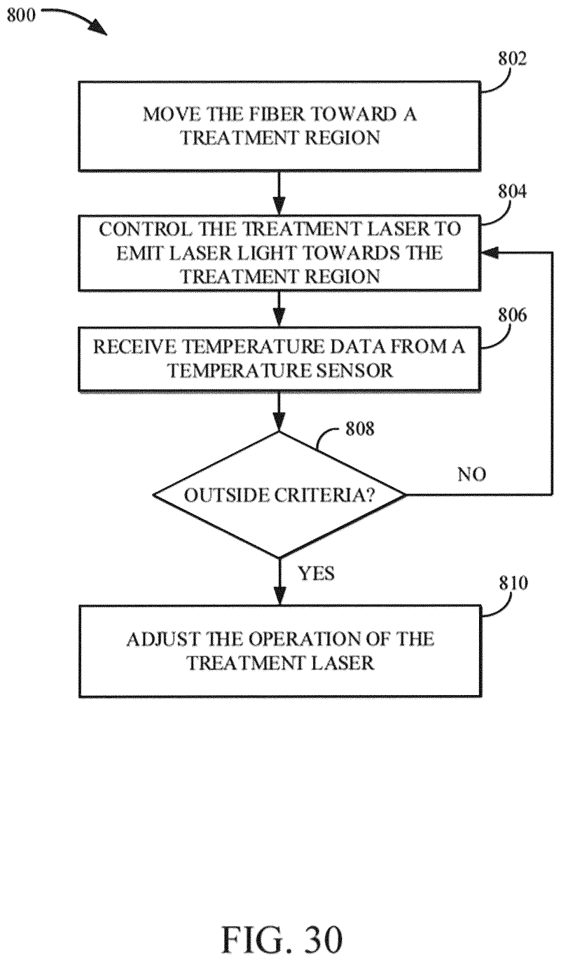

FIG. 31 shows a flowchart of a process 850 for determining a presence of a bubble within a treatment region. The process 850 can be implemented using any of the laser systems described herein (e.g., the laser system 100), and the process 850 can be implemented using one or more computing devices as appropriate (e.g., the computing device 130).

A first portion of laser radiation is completely absorbed by a water layer, which is vaporized as a result to create a bubble (vapor channel) between the fiber and the tissue (a phenomenon known as the “Moses effect”). The first portion of laser radiation should be optimized using several criteria, specifically: 1) to produce the minimal mechanical impact from increased water pressure induced by bubble formation; 2) to minimize the necessary laser energy to produce the vapor channel; 3) to produce a vapor channel with a pre-defined length; 4) to complete the first portion of radiation at the moment when the vapor channel reaches the surface of the target material or tissue; and 5) to complete the first portion of radiation at the moment when small fragments of stone cross the vapor channel during popcorning. Energy of first pulse portion is below or close to threshold of stone or tissue ablation.

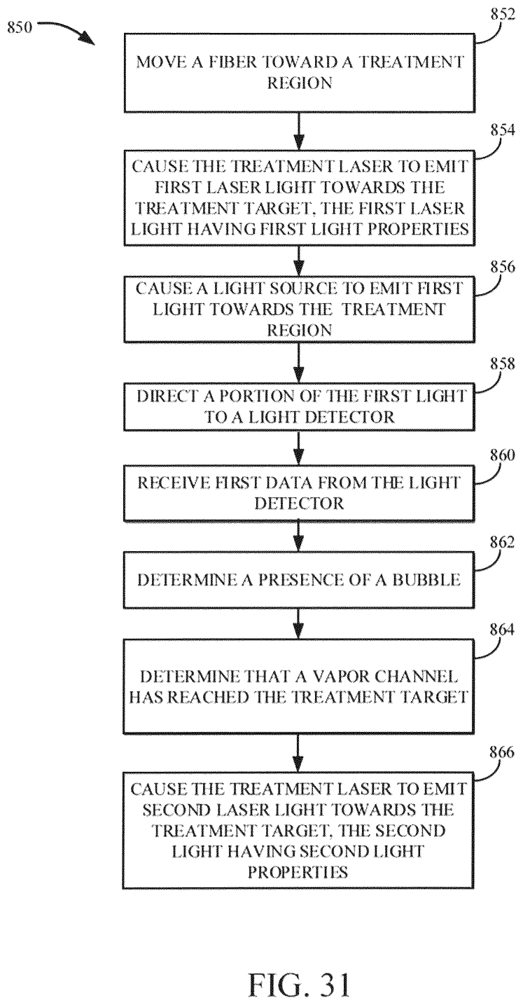

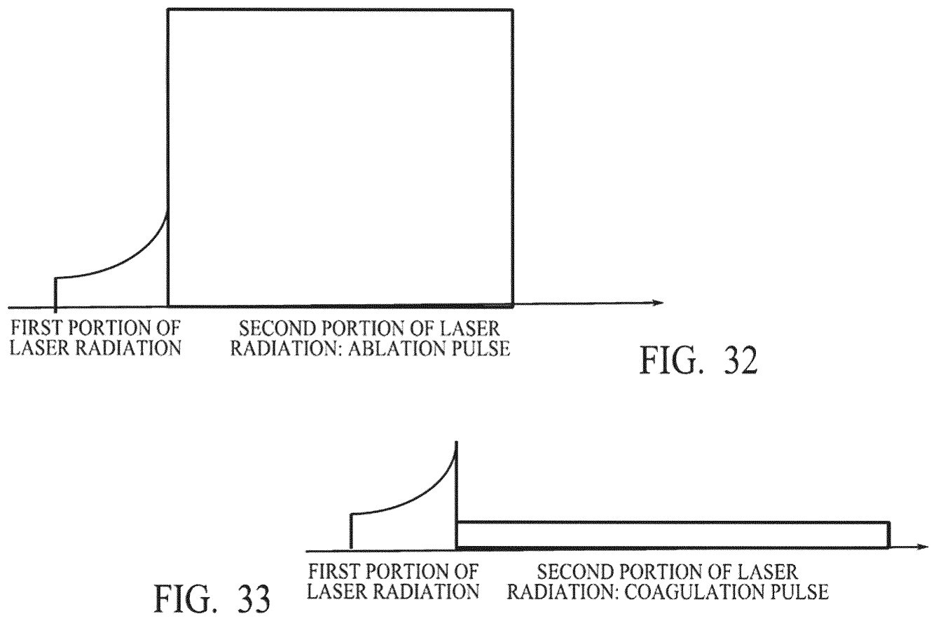

A second portion of the laser radiation starts almost immediately after completing the first portion (although in some instances there can be a short delay). The laser characteristics of the second portion, such as the power, the pulse shape, the pulse width (pulse length), and the energy are defined for the best tissue treatment effect. For treatments involving stone and tissue ablation, the pulse energy of the second portion of the laser radiation should be substantially higher than the threshold of ablation. A graph showing pulse energy characteristics of the first and second portions of laser radiation for ablation applications is shown in FIG. 32. In contrast to ablation, for soft tissue coagulation (hemostasis), the energy of the second portion should be higher than the threshold of coagulation, but lower than the threshold of ablation. A graph showing pulse energy characteristics of the first and second portions of the laser radiation for coagulation applications is shown in FIG. 33.

In accordance with certain aspects, conditions for the first portion of the laser radiation can be defined as follows:
1. The minimal mechanical impact from water pressure that is induced by bubble formation is required for purposes of decreasing retropulsion during stone treatment. The pressure in water during the bubble formation is minimal if the power density at the fiber distal end is not significantly higher than the threshold of water vaporization. According to some non-limiting examples, this ratio is in a range of 1.01 to 10, and preferably in the range 1.01 to 2. The optimal pulse width of the first portion of the laser radiation as well as the corresponding energy/fluence at the distal end of the fiber can be determined based on the maximum expected distance between the stone and the fiber distal end.
2. The minimum laser energy expenditure for creating a vapor channel with a desired length can be achieved when the bubble is not spherical but is elongated along the axis of the laser beam. This condition can be met with the same criteria as outlined in (1) above.
3. To produce a vapor channel with a desired length and minimal laser energy expenditure, a mechanism for growing the vapor channel can be employed. According to one non-limiting example, this mechanism includes maintaining a ratio between the laser power density at the front portion of the growing bubble (vapor channel) and the threshold of water vaporization within a range of 1.01 to 10, and preferably in the range of 1.01 to 2. The instantaneous power of the pulse of the first portion of the radiation can be increased to compensate for beam divergence. The first portion should terminate when the bubble achieves its desired length.
4. In a real clinical situation, the distance between the distal end of the fiber and the tissue or stone surface can be a value that varies over a wide range due to movement of the fiber. In accordance with one non-limiting example, an adaptive regime for the first portion of the laser radiation is implemented. For instance, back reflection of the optical radiation of the probing beam is measured before and during delivery of the first portion of laser radiation when the vapor channel is being created. The back scattering signal changes due to differences between the coefficient of back reflection corresponding to the boundary (interface) between vapor and water versus this boundary between vapor and tissue. When the detector (e.g., in the optical adapter) of the laser system registers this change, the laser is switched from the first portion of the laser radiation to the second portion of the laser radiation. In this mode, minimal energy and power of the first portion of laser energy is automatically achieved due to adjusting these parameters for each laser pulse.

During a popcorning mode of stone treatment, laser radiation is used for: 1) initiating a stream of water to move small (usually below 3 mm) fragments of stone, and 2) ablating (fragmenting) of the small fragments into even smaller fragments. The probability of ablating is very low due to the low probability of lasing in the moment when small fragments have moved close enough to the distal end of the fiber. In accordance with one non-limiting example, the laser is continuously fired with a low pulse energy and high repetition rate to produce water streaming. In addition, when a small fragment moves close to the fiber end, the coefficient of back reflection of the probing beam changes, which is detected by the sensor and control system of the laser system, and the control system controls the laser such that the laser is immediately fired with a high pulse energy to break the small stone fragments into even smaller fragments.

At 852 the process 850 can include a computing device moving a fiber to a treatment region that includes a treatment target, which can be similar to the block 602 of the process 600. At 854, the process 850 can include a computing device causing a treatment laser to emit first laser light towards the treatment region (e.g., and at the treatment target), which can be similar to the block 654. In some cases, the first laser light can have first light properties. For example, the first laser light can have a pulse that has a first leading edge, a first falling edge, and a first region between the first leading edge and the first falling edge. In some cases, the first falling edge can be greater than the first leading edge, and the first portion can have concave shape.

At 856, the process 850 can include a computing device causing a light source to emit first light towards the treatment region, which can be similar to the block 604 of the process 600. At 858, the process 850 can include directing a portion of the first light to a light detector, which can be similar to the block 606 of the process 600. At 860, the process 850 can include a computing device receiving data from the light detector, corresponding to the interaction between the portion of the first light and the light detector, which can be similar to the block 608 of the process 600.

At 862, the process 850 can include a computing device determining a presence of a bubble (e.g., at a distal end of the fiber), based on the data. For example, the first laser light can cause a bubble to form (e.g., by vaporizing liquid within the treatment region). Thus, a computing device can compare the data to a criteria (indicative of a bubble), and can determine that a bubble is present based on the data (e.g., one or more intensity values) exceeding a desired range or threshold (e.g., a criteria), and can determine that a bubble is not present based on the data not exceeding the criteria. In some cases, the process 850 can continue emitting the first laser light until the computing device detects a bubble. In some cases, if the computing device determines that the bubble is present the process 850 can proceed to the block 864.

At 864, the process 850 can include a computing device determining that a vapor channel has reached the treatment target (e.g., from the distal end of the fiber), based on the data. For example, the first laser light can create a vapor channel after the bubble has formed (e.g., the vapor channel being directed through the bubble). Once the vapor channel reaches the treatment target the reflection of light (e.g., scattering of light) can change, which can be used to determine that the vapor channel has reached the treatment target. Similarly to the block 862, the block 864 can include a computing device comparing the data to a criteria (indicative of a formed vapor channel in contact with the treatment target), and can determine that a vapor channel is present and in contact with the treatment target based on the data (e.g., one or more intensity values) exceeding a desired range or threshold (e.g., a criteria), and can determine that a vapor channel has not contacted the treatment target (or is not formed at all) based on the data not exceeding the criteria. In some cases, the process 850 can continue emitting the first laser light until the computing device detects a channel in contact with a treatment target (e.g., the process can proceed back to the block 854). In some cases, if the computing device determines that the vapor channel is in contact with a treatment target, the process 850 can proceed to the block 864.

In some non-limiting examples, after the computing device determines a presence of a bubble, the process 850 can proceed back to block 856 to emit different light (and acquire additional data, which can be analyzed according to the block 864).

At 866, the process can include a computing device causing the treatment laser to emit second laser light towards the treatment region (e.g., and at the treatment target), which can be similar to the block 654. In some cases, the second laser light can have second light properties different than the first light properties of the first laser light. For example, the power of the second laser light can be higher than the power of the first laser light, when, for example, the second laser light is configured to ablate the treatment target. As another example, the power of the second laser light can be lower than the power of the first laser light, when, for example, the second laser light is configured to coagulate the treatment target. As yet another example, the pulse width of the second laser light can be larger than the pulse width of the first laser light. In these ways, the first laser light can create the bubble, create the vapor channel, extend the vapor channel (within the bubble) until it contacts the treatment target, while the second laser light can pass through the vapor channel and can be directed at the treatment target. In this way, the treatment can be more efficient at least because laser light is not being undesirably just vaporizing liquid (and not being directed appropriately at the treatment target).

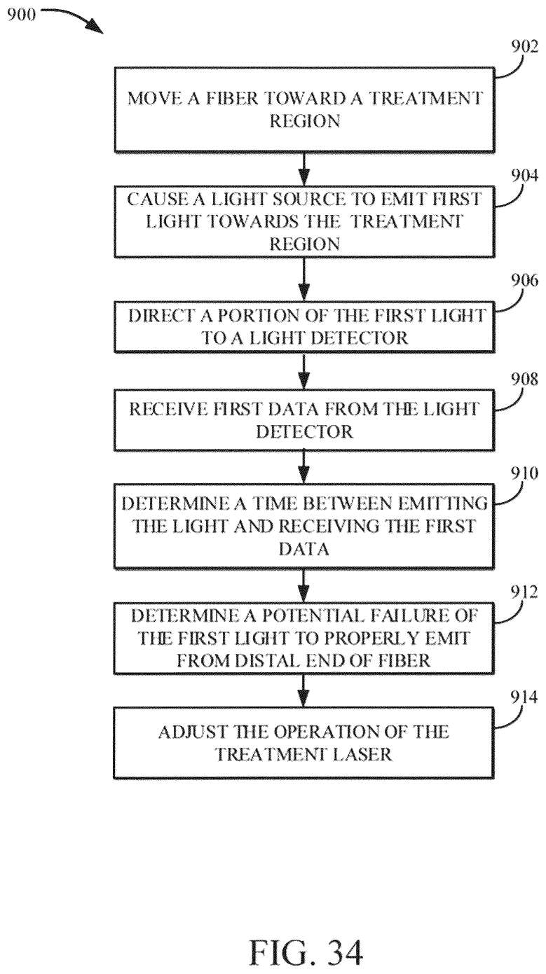

FIG. 34 shows a flowchart of a process 900 for detecting a problem with light transmission through the fiber. The process 900 can be implemented using any of the laser systems described herein (e.g., the laser system 100), and the process 900 can be implemented using one or more computing devices as appropriate (e.g., the computing device 130).

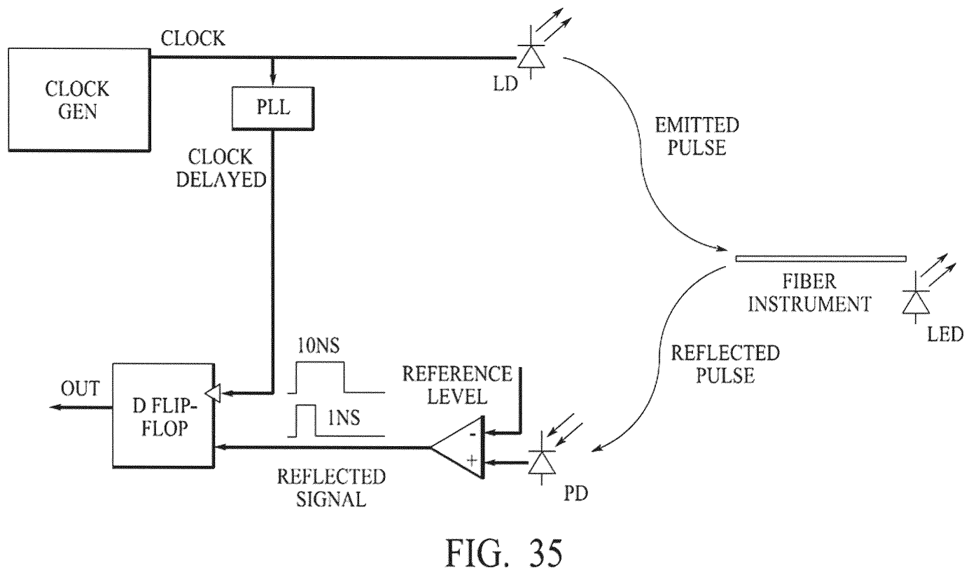

In accordance with at least one non-limiting example, FIG. 35 is one example of a functional schematic of the laser system. The synchronizing signal (CLOCK) originates in a clock generator. A laser source such as a laser diode emits a probe pulse on each arrival of the clock forefront. The period of pulses is greater than the longest time of flight of a pulse that goes from the beginning to the end of an instrument and then backward. The forefront duration can be less than 0.1 ns, and the pulse duration can be several nanoseconds. Low average power is ensured by a short pulse duration and a large period of pulses. If the power is insufficient for stable detection of a reflected signal, then the pulses are additionally amplified.

The laser emission is then coupled into the fiber instrument and partly reflected from the distal end of the fiber or a crack in the fiber. In instances where the cleave angle is too large at the distal end of the fiber, the reflected power can be too small to be detected. This situation is discussed in further detail below.

The reflected pulse is received by a sensor such as a photodiode. The signal is then digitized by a comparator, and the reference level of the comparator is then adjusted. In some instances, the target level of the comparator is half the amplitude of the pulse. Once it leaves the comparator, the reflected signal goes to the data input of a D flip-flop. A synchronizing signal is delayed in a phase-locked loop by a variable magnitude with a step less than 0.1 ns and goes to a clock input of the D flip-flop. The result of a comparison between these two inputs is latched at different moments of time, which makes it possible to fix the moment of transition of the result of the comparison from zero to one, and consequently to fix the forefront of the reflected pulse.

Two regimes of operation are possible. In the first regime, the delay of a latching clock is scanned step-by-step over all delay ranges that correspond to a length of a fiber instrument. Thus, the moment of pulse return is fixed and the fiber length is measured. In the second regime, the range of delays that correspond to a small zone around the tip of the fiber instrument is employed. In this instance, the reflection of a pulse from a tip is fixed. If the reflected pulse disappears, two variants are possible: a crack inside a fiber, or a very large cleave angle at the tip of the fiber instrument. Both situations demand immediate termination of laser emission. Operation of a device in the first regime gives detailed information about the fiber instrument, but the second regime takes less time.

In accordance with a further aspect, light transmitted through the surgical fiber can be used to detect fiber breakage. Any source of illumination from the treatment area can be used, e.g., an LED built into the endoscope. The power of the LED can be measured by the same photodiode as the photodiode used to detect the reflected pulse signal as described above. A decrease in the measurement result indicates problems with the integrity of the fiber. In some instances, the two types of measurements described here can be performed in turn.

To summarize, fiber breakage and its position can be detected using light emission from two sources: laser radiation from a laser probe source and an LED source located in the treatment area. If used together with the main surgical laser radiation, the reflected probe signal can be spectrally separated and filtered by the system to monitor and react to fiber breakage.

At 902, the process 900 can include a computing device moving a fiber to a treatment region that includes a treatment target, which can be similar to the block 602 of the process 600. At 904, the process 900 can include a computing device causing a light source to emit first light towards the treatment region, which can be similar to the block 604 of the process 600. At 906, the process 900 can include directing a portion of the first light to a light detector, which can be similar to the block 606 of the process 600. At 908, the process 900 can include a computing device receiving data from the light detector, corresponding to the interaction between the portion of the first light and the light detector, which can be similar to the block 608 of the process 600.

At 910, the process 900 can include a computing device determining a time between emitting the first light and receiving the data. For example, a computing device can determine a first time (e.g., create a time stamp) when the first light is emitted by the first light source, and a computing device can determine a second time (e.g., create a time stamp) when the data is received. Then, a computing device can determine a difference between the first time and the second time.

At 912, the process 900 can include a computing device determining a problem with light transmission through the fiber (e.g., based on the data). For example, a computing device can compare the time (e.g., from the block 910) to a desired time, and based on the time being lower than the desired time the computing device can determine the problem, whereas if the time is higher than the threshold time the computing device can determine that there is no problem. A lower time (e.g., lower than expected) between the emission and receiving can indicate that light is entering the fiber at an undesired location (e.g., away from the distal end).

In some non-limiting examples, a computing device can determine the problem by comparing the data to a criteria, and if the data (e.g., an intensity value) is less than the criteria then a computing device can determine that a problem exists (e.g., light is not being properly delivered through the fiber). Alternatively, the computing device can determine that a problem does not exist if the data is greater than the criteria.

At 914, the process 900 can include a computing device adjusting the operation of the treatment laser, and notifying a practitioner, each of which can be based on the problem having been determined (e.g., at the block 912). The block 914 can be similar to the block 762 of the process 750. For example, a computing device can cause a treatment laser to stop emitting laser light, can disable (temporarily) the treatment laser from emitting the laser light, etc., based on the problem being determined at the block 914. In some cases, the problem can be a break, a kink, a bend, etc., in the fiber.

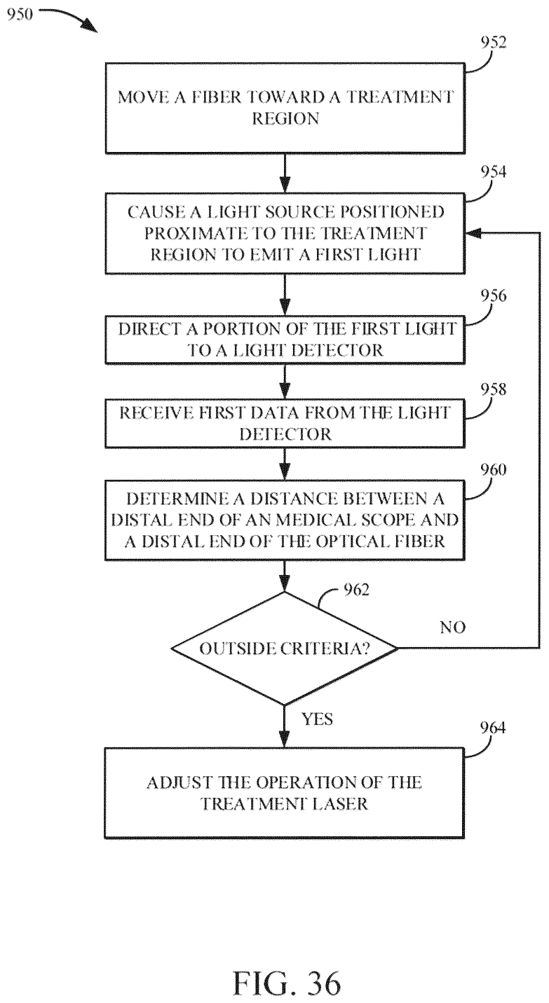

FIG. 36 shows a flowchart of a process 950 for determining a distance between a distal end of a fiber and a distal end of a medical scope (e.g., a channel of a medical scope that receives the distal end of the fiber). The process 950 can be implemented using any of the laser systems described herein (e.g., the laser system 100), and the process 950 can be implemented using one or more computing devices as appropriate (e.g., the computing device 130).

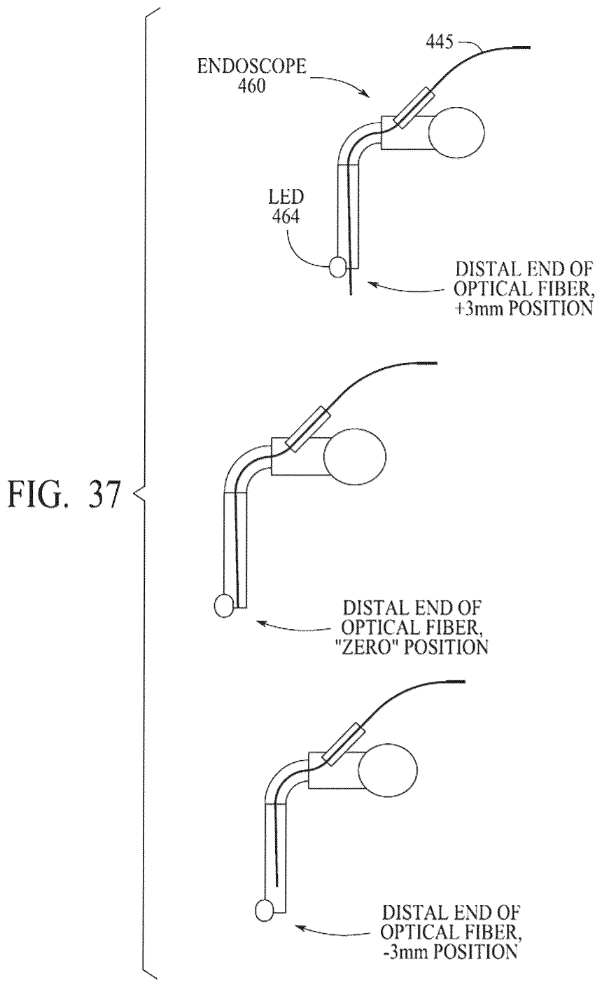

Another example of a laser system with different surgical fiber tip positions relative to the output/end of the endoscope is shown in FIG. 37. As mentioned previously, the surgical fiber 445 is inserted into the endoscope. The proximal end of the fiber 445 is connected to the optical adapter, which is part of the smart laser system as previously described. The amount of light that gets into the fiber from the LED source of scope 460 due scattering in treatment area on liquid and wall of organ is measured and analyzed by detector and the control system of the laser system. The measured signal is extremely sensitive to the position of the distal end of the fiber with respect to the distal end of the endoscope.

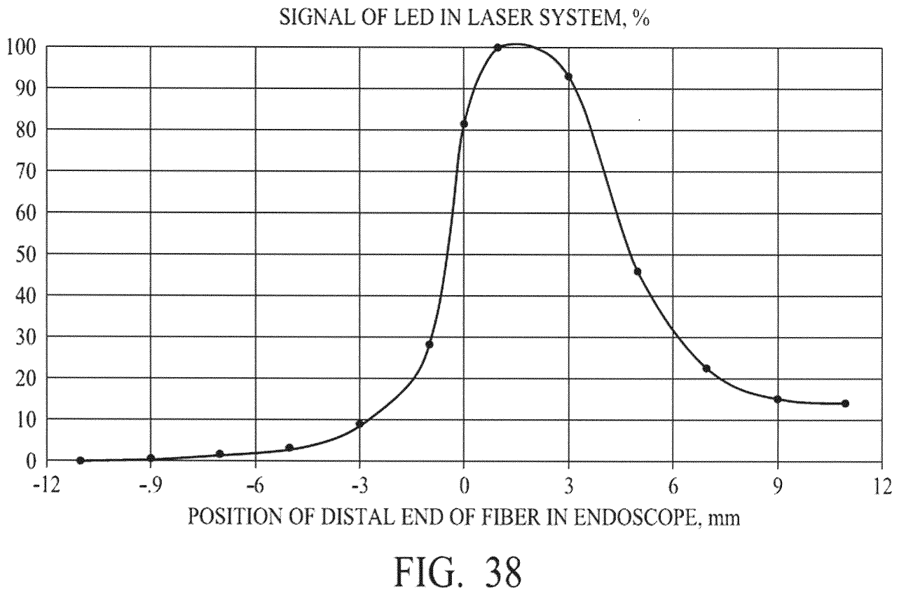

FIG. 38 shows a typical dependence of the LED signal measured by the laser system on the position of the distal end of the fiber. In this instance, 0 mm corresponds to the position of the distal end of the fiber at the distal end of the endoscope, which is also shown as “zero” position in FIG. 37. This method can be used by the system or a user to control the position of the distal end of the fiber with respect to the end of the endoscope to be within a predetermined and optimal range, for example, from 0 to 3 mm (e.g., as a correct position of the distal end of the fiber). The system is configured to control the laser source such that high power laser radiation is not emitted when the distal end of the fiber is in the incorrect position. This reduces or eliminates risk of 1) endoscope shaft damage if distal fiber tip inside working channel of endoscope and user actives treatment laser operation using foot pedal. Control system will block laser operation if fiber distal end inside scope position is detected. 2) potential fiber damage if distal fiber end is extending more than 3-5 mm from scope, 3) accidental fiber breakage inside scope and following damage of scope shaft due to treatment laser emission inside working channel.

At 952, the process 950 can include a computing device moving a fiber to a treatment region that includes a treatment target, which can be similar to the block 602 of the process 600. At 954, the process 900 can include a computing device causing a light source positioned within the treatment region to emit first light towards the treatment region. The block 954 can be similar to other blocks (e.g., 904) of other processes described herein. At 956, the process 950 can include directing a portion of the first light to a light detector, which can be similar to the block 606 of the process 600. At 958, the process 900 can include a computing device receiving data from the light detector, corresponding to the interaction between the portion of the first light and the light detector, which can be similar to the block 608 of the process 600.

At 960, the process 950 can include a computing device determining a distance between a distal end of the medical scope and a distal end of the fiber, based on the data. For example, a computing device can compare the data to a curve (e.g., the curve of FIG. 38) that relates the intensity value of light (e.g., the first light) relative to the position of the distal end of the fiber relative to the distal end of the medical scope to determine the distance.

At 962, the process 950 can include a computing device determining whether or not the distance (determined at the block 960) exceeds a distance criteria. If at 962, the computing device determines that the distance exceeds (e.g., is greater than, or in some cases is less than) the distance criteria, then the process 950 can proceed to the block 964. However, if at 962, the computing device determines that the distance does not exceed the distance criteria, then the process can proceed back to the block 954 (or the block 952. In some cases, when the distal end of the treatment laser is located within the medical scope, it is not desirable to fire the treatment laser (e.g., because it could damage the internal components of the medical scope). In addition, when the distal end of the treatment laser is located too far from the distal end of the scope, the laser light may not be properly delivered. In some cases the distance criteria can be substantially −1 mm, 0 mm, 1 mm, 3 mm, etc.

At 964, the process 950 can include a computing device adjusting the operation of the treatment laser, and notifying a practitioner, each of which can be based on the distance having exceeded the criteria determined (e.g., at the block 962). The block 962 can be similar to the block 762 of the process 750. For example, a computing device can cause a treatment laser to stop emitting laser light, can disable (temporarily) the treatment laser from emitting the laser light, etc., based on the distance having exceeded the criteria.

Thus, a variety of system and methods have been provided. In some non-limiting examples, calculi (e.g., kidney stones, ureter stones, etc.) and soft tissues have different structures (and material properties) that create specific responses by a probing light (e.g., light from one of the light source(s)). For example, different stones can have different chemical compositions, with stones in general mostly containing minerals with water present in the inter-crystalline and microcrystalline spaces (e.g., about 10% of the total volume of the stone). In addition, stones can contain small organic molecule additions, and each stone can have different micro structures, macro structures, shapes, surface structures, and conditions. Each of these can define the stone's optical properties, which can include a spectrum of the absorption, a spectrum of the scattering coefficients, the angle distribution of scattered light, etc. In some cases, the stones backscatter the probing light, which results in different types of scattering (e.g., Rayleigh, Mie, etc.). In contrast to stones, tissues (e.g., kidney, ureter, soft tissues, etc.) can include an organic extracellular matrix, vascular systems, and cells. Aside from the substantial difference in water content between tissues and stones (e.g., tissues containing substantially 70-80% water, while stones can contain substantially 10% water), tissues can have a non-porous structure and smoother surface (as compared to stones). As a result, tissues have different optical properties than stones. For example, tissues can scatter light less than stone material (e.g., in most conditions), especially in the ranges of wavelengths where there is significant water or blood absorption (e.g., the light being absorbed by the tissues and thus not scattering). Thus, the light directed back into the distal end of the fiber can be used to determine (e.g., by a computing device) if the treatment target (or other structure near the fiber) is stone, or is tissue. In addition, the light directed back into the distal end of the fiber can even discern the type of stone, if, for example, the treatment target has been determined to be a stone.

In some non-limiting examples, when the probing light approaches the treatment target (or other structure near the fiber), the amount of the light directed back into the distal end of the fiber increases (e.g., the probing light reflecting off the target and being directed into the distal end of the fiber). For example, when the distal end of the fiber 108 is in contact with the treatment target (in some instances the gap is about 100 microns or less), the amount of the light directed back into the distal end of the fiber (e.g., derived from a light source that emits the probing light) is maximized at least because more of the light is directed back into the fiber rather than being dissipated within the treatment region. This response, however, can be different for stones than for soft tissue because the backscattered amount of light from the stones and from soft tissue differs, especially in the wavelength ranges where blood and/or water absorption occurs (e.g., because the tissue absorbs more of this light that stones, and thus a larger amount of back-reflected light in these wavelengths occurs for stones). The probing light (e.g., which can be a laser beam) can have several wavelengths where a contrast between the back-reflected signal of the stone and the tissue (e.g., soft tissue) is maximized. In some non-limiting examples, the probing light can be a broad continuous spectrum source (e.g., the probing light having one or more wavelengths within a range of substantially 400 nm to substantially 750 nm), such as an LED or a lamp for purposes of obtaining a broad spectrum back-reflected signal from the tissue or stone.

In some non-limiting examples, by establishing or otherwise determining certain thresholds, desired ranges or above or below limits of the light directed back into the distal end of the fiber (e.g., the intensity of the light directed back into the distal end of the fiber light, from, for example, the probing light emitted) that correspond to contact (or quasi-contact) with the treatment target (e.g., stone verses tissue), a computing device can detect when the fiber is in contact with the treatment target. For example, a computing device can receive data from a detector (e.g., one of the light detector(s)) that detects the backscattered light (e.g., the light directed back into the distal end of the fiber) and can determine a distance between a distal end of the fiber and the treatment target based on the data (e.g., an analysis of the data), which can include determining whether or not the fiber is in contact with the treatment target. This process can be implemented in real time (e.g., relative to a practitioner) so as to provide (and present on a display) a current distance between the distal end of the fiber and the treatment target so that the laser system (or practitioner) can adjust control of the treatment laser accordingly. In some non-limiting examples, the process of detecting the backscattered light (if any) from the distal end of the fiber and its comparison to a limit or range (e.g., one or more reference value(s) based on data from urine, water, air, or the like, without the presence of the treatment target, or based on data from a surgical component such as a catheter, a basket, a stent, a sheath, etc.) can allow the laser system to determine the approach to the target, to detect contact with the target, and the ability to distinguish whether the target is a stone (of some kind), a tissue, or a surgical component.

In some non-limiting examples, when the fiber is in contact with or not farther than a predetermined distance (e.g., 1 mm) from the treatment target or surgical component(s), the practitioner (or a computing device) can turn on the treatment laser or increase the laser power/energy of the laser treatment light to treat the treatment target. In some cases, when the fiber is in contact or close to a surgical component, the practitioner (or a computing device) can turn off the treatment laser or decrease the power of the laser treatment light to prevent laser damage of surgical components. In some configurations, when the distance is farther than a desired range or limit, or when the treatment target is target material but the portion of the subject is determined to be tissue and the treatment is not intended to ablate or coagulate soft tissue, the laser system (e.g., the computing device) can notify the practitioner to prevent emission of the laser treatment light. In some cases, then, the laser system (e.g., a computing device) can turn on the treatment laser, turn off the treatment laser, change the power of the laser treatment light, change the energy of the laser treatment light, etc., based on the data from the light directed back into the distal end of the fiber (e.g., the backscattering light).

In some non-limiting examples, when a bubble (e.g., an air bubble) is formed in front of the fiber in the treatment region, the laser system can detect or otherwise determine whether the reflected light (e.g., that is the light directed back into the distal end of the fiber) is from an interface (or in other words border) between air and water in front of the (growing) bubble (e.g., when no tissue or stone is within the bubble). In addition, the laser system can determine that the reflected light is within the volume of the bubble (e.g., between air and the tissue or stone including when the tissue or stone is within the bubble). In some instances, the probing light can be the laser treatment light (e.g., that is the light directed back into the distal end of the fiber), while in other cases the probing light can be from other light sources (e.g., one of the light sources). In some cases, the probing light can have a wavelength specifically selected for a high resolution for the two scenarios above (e.g., the reflected light outside and within the bubble).

In some non-limiting examples, the laser system can be configured to detect a breakage (or inability to properly emit laser light) by using principles of laser reflectometry. For example, a laser (e.g., the treatment laser, or one of the other light sources) can emit short probe pulses of light into the fiber, which are reflected from a distal end of the fiber, or at a crack inside of the fiber. A detector (e.g., one of the light detector(s), such as a photodiode, can receive the reflected light pulse, and a computing device can determine the delay relative to the initial pulse. This delay is unambiguously associated with a distance from the laser to the point of reflection and as described in further detail below, can be used by the system to detect fiber breakage (or can detect a state of the fiber in which the fiber is not able to properly emit laser light from the distal end). In some cases, the detection of fiber breakage can also be based on simply sensing a light signal transmitted through the surgical fiber. In this instance, any source of illumination of the treatment area (e.g., an LED, lamp built into the endoscope, etc.) can generate light and a detector such as a photodetector on the laser system can sense light transmission through the fiber (or a lack thereof).

In some non-limiting examples, the laser system (and others described herein) can be calibrated to certain desired ranges or levels of the analyzed data, or data combinations (e.g., data from more than one detector). This can lead to the ability to either interrupt operation of the treatment laser, or give a warning to the practitioner (e.g., audio through via a speaker, a visual signal presented on a display, etc.) and propose a further action. In some non-limiting examples, either scenario can require the user's input as to whether to continue treatment (e.g., continuing delivering the laser treatment light), adjust operating conditions (e.g., adjust the treatment laser operation parameters, move the fiber 108, pause the delivery of the laser treatment light for a period of time, etc.), or to ignore the system recommendations.

In some non-limiting examples, the laser system can be calibrated within a particular clinical environment prior to treatment, or can even be configured to self-calibrate during a clinical procedure. The laser system can also be configured to accumulate feedback signals (e.g., data from the light directed back into the distal end of the fiber) and analyze patterns and classify them based on particular characteristics and reactions of the user to increase the potential for “smarter” responses and recommendations. In some cases, then, the laser system can function semi-autonomously, autonomously, etc.

In some non-limiting examples, the laser treatment light from the treatment laser, the light from each light source(s), and the light received by each light detector can be implemented in different ways. For example, the laser system can include one or more fiber optical couplers to facilitate light from each light source reaching the fiber, laser treatment light from the treatment laser reaching the fiber, and light from the fiber 108 reaching each light detector. For example, the laser system can include a N×1 tree coupler (i.e., a first tree coupler) with N inputs and 1 output, in which each of the N inputs can be in optical communication with a respective light source(s), and the 1 output can be in optical communication with the fiber (e.g., coupled to the proximal end of the fiber). Correspondingly, the laser system can include a 1×N tree coupler (i.e., a second tree coupler) in which the 1 input can be in optical communication with the fiber (e.g., coupled to the proximal end of the fiber) and each of the N outputs can be in optical communication with a respective light detector. In some cases, the laser system can include a 3×1 tree coupler (i.e., a third tree coupler) in which a first input of the three can be coupled to the output of the first tree coupler, a second input of the three is coupled to the input of the second tree coupler, and a third input of the three is coupled to the laser fiber (e.g., that directs the laser treatment light). Then, the output of the third tree coupler can be coupled to the proximal end of the fiber. In this way, each light source, each light detector, and the treatment laser can be in optical communication with a respective fiber, each of which can be coupled to the fiber. Thus, each respective fiber can define a different optical path for light emitted by each light source, the treatment laser, and for light received by each light detector. While this is only one example, others are contemplated for routing the different optical channels to (and from) the fiber. For example, a multicore optical cable can be in optical communication with the fiber, with each channel of the multicore optical cable being in optical communication with a respective light detector. Similarly, a multicore optical cable can be in optical communication with the fiber, with each channel of the multicore optical cable being in optical communication with a respective light source.

Additional System Features to Facilitate Treatment of Tissues and Calculi with Directed Energy

The use of directed energy is increasingly a method of choice to treat various pathological conditions of the human body. Different kinds of directed energy are known in the art: electromagnetic (ranging from X-ray to RF), mechanical (including directed particles—e.g., electrons or protons), acoustic (including ultrasound), and others. Critical to the success of the treatment is the ability of the operator to correctly target the desired pathology while minimizing collateral damage to surrounding intact tissue and ensuring overall patient safety.

In accordance with various aspects, one objective is to provide advanced techniques to assist the operator in achieving these goals based on registration and interpretation of various diagnostic information obtained from the prospective treatment sites and surrounding areas. Most of the embodiments disclosed herein deal with laser lithotripsy of urinary calculi; however, applications directed to other conditions, body areas, and forms of directed energy can be readily made by those skilled in the art and are included within the scope of this disclosure.

Laser lithotripsy is a well-known and effective way of treating urinary stones. Laser energy is delivered from a laser to the distal end of the optical fiber inserted into an endoscope (including, but not limited to, flexible, semi-rigid, and rigid scopes). The distal tip of the fiber is placed in front of the target, the laser is fired, and absorption of the laser energy by the stone target leads to destruction of the stone. Various techniques (e.g., fragmentation, popcorning, and dusting) for delivering the laser energy are known in the field. Once the target stone is split into sufficiently small fragments, these fragments can be passed naturally. Alternatively, they can be removed using auxiliary tools (e.g., baskets) or aspirated through the working channel of the scope.

Conventionally, the operator (e.g., surgeon or physician) identified the target as a stone by using a monitor that displays a real-time picture received from a built-in endoscope camera with the target area illuminated by the endoscope LED or other source. However, this technique poses potential problems concerning the field of view of the camera. For example, the doctor still can experience difficulties viewing the target while the laser is firing or right after the laser stops firing since there are multiple processes that take place: bubble and microbubble formation at the distal tip of the fiber, and blocking of the image of the stone or tissue due to scattering of the illumination light off the product of stone ablation. During such processes stone dust tracks in all directions, which leads to scattering of the illumination provided by the LED or other light source and to other energy source scattering. The camera's field of view becomes more and more obscured (polluted) after the laser is turned on, and eventually in many cases, it becomes impossible for the doctor to determine the target by using only the image from the camera. In these moments the doctor is basically blind and presents a possible collateral risk of soft tissue damage during this timeline of the treatment. To avoid this, the doctor usually stops firing and temporarily increases fluid flow in order to clear the field of the view of the camera. Another problems is that the stone can be in close contact with soft tissue, such as, for example, a stone in the ureter. When a surgeon is treating such a stone in a scanning mode (dancing mode) and moving to the edge of the stone, the edge of the stone is in contact with a mucosal surface and it is very difficult to prevent the laser from firing on the mucosal tissue. This can result in thermal damage to the ureter wall, which can result in scar tissue formation and ureteral stricture, i.e., a narrowing of the ureteral channel (stenosis). When a narrowing in the ureter occurs, the kidney cannot function normally and will be damaged over time. Treatment for ureteral stricture may include surgical implantation of a stent to open the narrowed section of the ureter or surgery to reconstruct the urinary tract.

The unwanted result in these instances is that the treatment and anesthesia time are prolonged, which can be crucial in some cases. The most severe cases can lead to perforation of the kidney or ureter wall and eventually to unwanted urgent kidney or ureter surgery being required. The procedure should prevent mucosal tissue perforation and collateral damage of the wall of the bladder, kidney, or ureter. Furthermore, if there is a gap between the distal tip of the fiber laser and the stone (i.e., the fiber tip and the stone are not in direct contact with one another), e.g., more than about 0.5 to 1.5 mm, then the ablation efficiency will be less than the theoretical maximum or be less than what is otherwise potentially attainable (when compared to a desirable fiber position scenario) due to laser beam attenuation, which increases the treatment time as well.

Ensuring contact of the laser fiber with the stone prevents water overheating and the active use of the popcorning mode leads to better stone fractioning and prevents unintended firing on soft tissue. The possibility of adding additional assistance to the doctor (beyond the view from the camera) for purposes of detecting contact with stone or soft tissue and distinguishing between these surfaces (to be confident of the correct position of the distal tip of the fiber) as a part of a smart (intelligent) laser system would result in a substantial increase in such a system's value and importance. In alternative embodiments that include treatment configurations where contact with soft tissue is desired (e.g., tumor treatment) and when a clear image from the field of view of the camera is difficult or complicated to obtain, providing information about whether or not the fiber position is in contact with soft tissue can lead to shortening and optimization of the procedure as well.

In accordance with at least one embodiment, means for identifying the target are provided and, in embodiments where a fully automatic mode is employed, the controlled emission of the directed energy is implemented. This allows for the laser lithotripsy treatment to be more efficacious, safe, and convenient for the doctor and minimizes procedure time and the time needed for learning the laser lithotripsy technique.

According to certain embodiments, an operational principle is based on the differences between the light reflection coefficients (or other energy reflection properties) of stones, soft tissues, ambient medium (water), and, potentially, other objects (e.g., surgical components) present in the treatment field (e.g., surgical instruments, stents, etc.) for selected wavelength bands. The reflection coefficient is defined within the context of the directed energy used. In some embodiments, the wavelength(s) of the optical energy (and its reflected wavelength) is in a range of 200 to 11,000 nm, preferably in a range of 300 to 2,700 nm, and most preferably in a range of 400 to 1,200 nm. The absolute values of the reflected intensity signals may vary widely depending on the illumination conditions (typically, an LED in the scope), as well as the signal acquisition conditions. Hence, in accordance with various embodiments a ratiometric approach is used to classify the target. Furthermore, a priori knowledge about the reflective properties of the materials involved may not be sufficient for reliable system calibration, in which case individual in-patient calibration is required.

Per one or more aspects of the ratiometric approach, the ratios of the reflected signal intensities at multiple wavelength bands may be combined in different ways to yield optimal separation (i.e., distinction) between the classes of objects of interests (primarily, stones and soft tissues). For example optical data can be generated that corresponds to the reflected light intensity and this can involve determining at least one ratio of a reflected light intensity of one selected wavelength band to reflected light intensity of a different selected wavelength band. The ratiometric technique can also be used in a calibration routine, as discussed further below. The thresholds of the firing decision (enable/disable or adjust the directed energy emission) can also be set in several ways. Finally, the system may provide informational feedback to the operator regarding the class of the target through various sensory means.

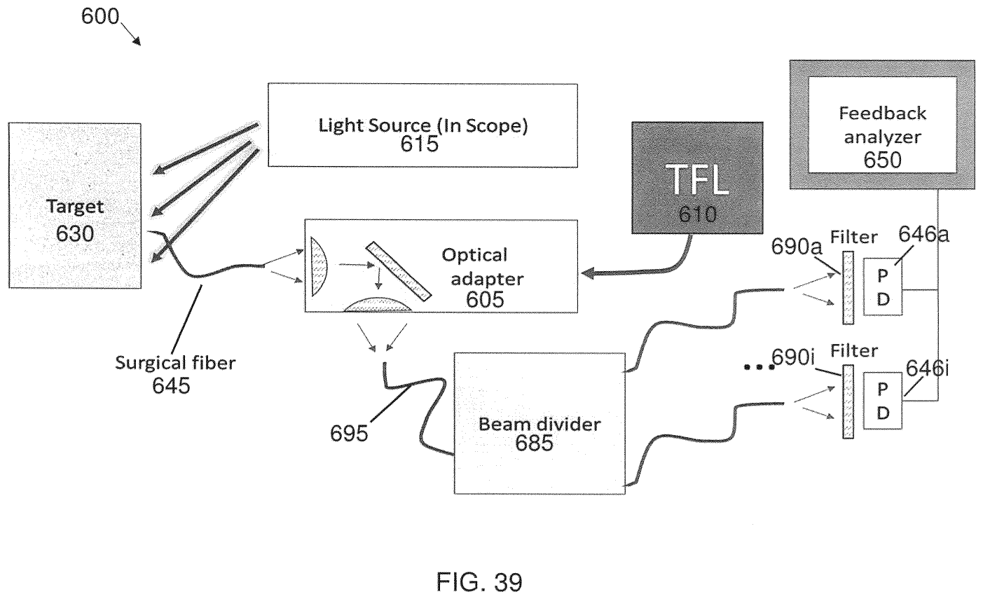

One example of an intelligent/smart system 600 is shown in FIG. 39 in accordance with at least one embodiment. System 600 comprises a laser system with a surgical fiber 645, an endoscope (in at least one instance the surgical fiber is at least partially incorporated into the endoscope), and a reflected signal analyzer module or feedback analyzer (i.e., controller or computing device). This system does not use a spectrometer.

The treatment laser or laser source 610 is a fiber laser, solid-state laser, or other type of treatment laser. One non-limiting example of a fiber laser includes a thulium fiber laser (TFL) as shown in FIG. 39. Lasers sources emitting energy at other wavelengths are also within the scope of this disclosure. The laser radiation emitted from the laser source 610 is directed through the endoscope and directed at the target 630. The laser radiation emitted from the laser source 610 is also delivered to the optical adapter 605, as previously described.

A (broadband or selected band) source of light 615 is built or otherwise integrated into the endoscope (typically, LED source; however, low power lasers, laser-pumped fluorescent lamps, LED-pumped fluorescent lamps, thermal sources, such as xenon, halogen, etc. lamps are also possible) and is configured to illuminate the field of operation/manipulation (surgical treatment area) inside the patient.

The detection arm of system 600 implements the use of optical fiber 645 (also referred to herein as surgical fiber), the optical adapter 605, an interconnecting fiber 695, a beam divider 685, a feedback analyzer 650 (also referred to herein as a computing device), optical filters (e.g., 690a-690i) and corresponding photodetectors (e.g., 646a-646i).

As discussed in further detail below, the surgical fiber 645 is configured to receive light (e.g., light from light source 615) that is reflected from the target 630 in a surgical treatment area. The computing device 650 is configured to couple with at least two of the photodetectors 646a-646i. Each photodetector 646 is configured to detect an intensity of reflected light from the target 630 in a different selected wavelength band. As also discussed in further detail below, the computing device 650 is configured to receive the reflected light intensity in at least two (different) selected wavelength bands, generate optical data corresponding to the reflected light intensity, and identify the target as a treatment target or a non-treatment target based at least in part on the optical data and a predetermined calibration based on at least two known targets.

In accordance with various embodiments, the detection arm is capable of implementing several (e.g., up to 5) wavelength-specific channels yielding reflected signal intensities. For example, reflected signals from the treatment area are directed through the surgical fiber 645, interconnecting fiber 695, and into the optical adapter 605, which directs them to the beam divider 685, which splits the reflected signal into respective channels that each include an optical filter 690 and a photodiode or other type of photodetector 646. The filter 690 transmits reflected signal wavelengths of interest (i.e., predetermined wavelengths) to the photodiode 646, which is used in the analysis performed by the feedback analyzer or computing device 650 (which can be a computer processor as known in the art). In addition, system 600 may also comprise an endoscope camera (not shown in FIG. 39) that translates the real-time picture of the surgical treatment area field of view through to an outside monitor/screen that can be viewed by the operator. In some embodiments, the camera can be used in combination with an analyzed signal from the smart sensor system (i.e., analyzed data obtained from the detection arm(s)), to help the operator guide the surgical fiber 645 in the treatment field. It is to be appreciated that according to some embodiments, a portion or portions of the optical train between the interconnecting fiber 695 and the detectors 646 can be implemented as free-space optics.

Since the light source 615 is typically broadband, the wavelength selection is performed in the detection arm via the optical filters 690a-690i. According to one embodiment, the spectral bands may be selected from the following list (although it is to be appreciated that this list is exemplary and that other wavelengths may be used as well): about 400-410 nm, about 440-480 nm, about 460-480 nm, about 510-530 nm, about 540-560 nm, about 550-570 nm, about 570-580 nm, about 580-600 nm, about 600-620 nm, about 690-710 nm, about 740-760 nm, about 790-810 nm, about 920-940 nm, about 970-990 nm, and about 1150-1350 nm.

In accordance with various aspects, the wavelengths up to 620 nm are related to or are otherwise associated with hemoglobin absorption (soft tissues contains hemoglobin but stones do not), whereas wavelengths longer than 620 nm are sensitive to differences in scattering properties as well as to absorption by other chromophores. While the center wavelengths of the detection bands lie within the above ranges, the spectral widths of the detection bands (FWHM) may vary between 1 and 50 nm (preferably, 10 to 30 nm).

One or more varieties of the signal detectors or photodetectors (e.g., photodiode 690) may be used, e.g., multi-pixel photon counters (MPCCs), photomultiplying tubes (PMT's), or photodiodes (PDs, with a broad inclusion of these types of devices, including avalanche and PIN PDs, preferable).

There are several techniques that are suitable for separating the reflected signal into individual wavelength channels. In one embodiment, the total signal is split into N channels using a fiber splitter, with subsequent spectral filtration of each individual channel (e.g., the use of a beam divider, such as beam divider 685 as shown in FIG. 39). In other embodiments, the wavelength separation can be performed by using a set of beamsplitters (and configured with wavelength-selective coatings), prisms, cubes, or other similar wavelength-selective elements with subsequent detection of the respective wavelength bands at individual channels (by the detector, such as the photodiode 646 (PD)).

### Calibration

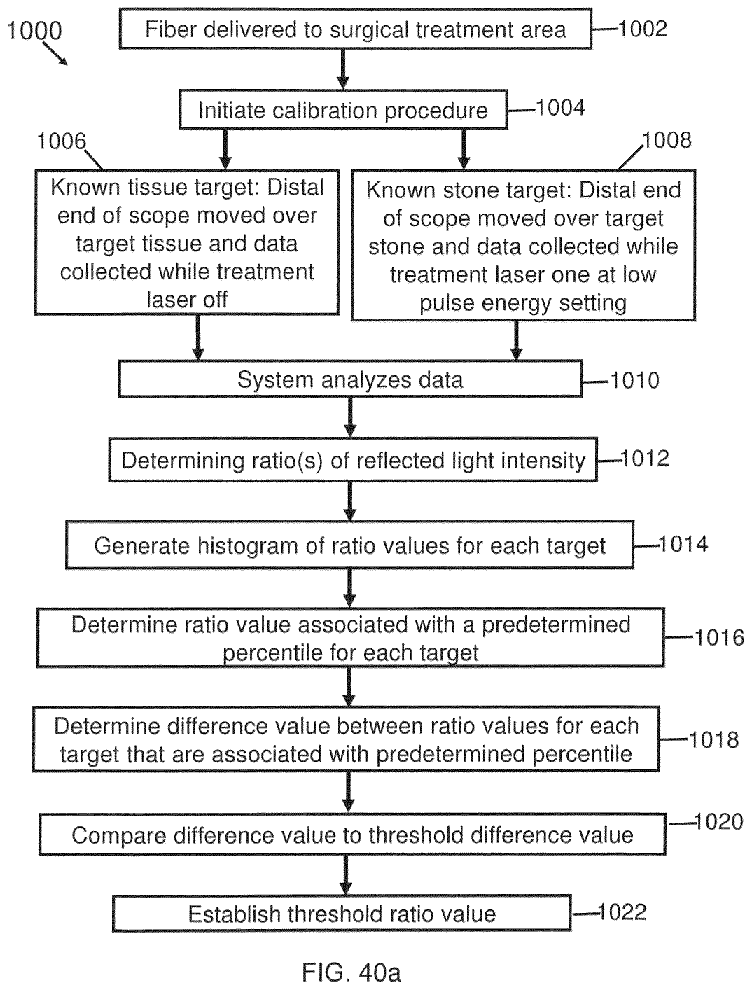

While some embodiments may implement the use of pre-programmed calibration tables or use a pre-procedure device calibration on phantom materials, at least one embodiment utilizes an in-patient calibration performed immediately prior to the actual procedure. The calibration (also referred to herein as a predetermined calibration) may be performed at the start of each procedure (e.g., for each patient). An exemplary overall process flow of the in-patient (predetermined) calibration is shown in FIG. 40a. Applicant discovered that clinical studies performed using the pre-treatment in-patient calibration (described in further detail below) provided significant improvement in the clinical performance and safety of the treatment.

FIG. 40a shows a flowchart of a process 1000 for an in-patient calibration procedure according to one embodiment. The process 1000 can be implemented using system 600. At 1002 the process 1000 can include delivering the fiber to a surgical treatment area. This may include inserting the fiber into a scope and delivering the scope to the treatment area. Step 1002 may be performed by a surgeon or a device configured to perform this step. At 1004 the calibration procedure initiates. Using the image from the FOV of the camera positioned on the distal end of the scope, the doctor positions the tip in front of various known target materials and obtains reflected intensity values from the photodetectors 646 that are coupled to the computing device 650.

According to at least one embodiment, multiple reflected light intensity values are obtained from each known target of at least two known targets. In one embodiment, the two known targets may be a stone and tissue. For example, the doctor or operator may position the distal end of the fiber tip in front of a stone (e.g., using the image from the camera to identify the stone) and obtain multiple reflected light intensity values (from each photodetector 646) of the light from light source 615 as it is emitted from the distal tip of the scope and reflects off the stone material and reflected light is captured by surgical fiber 645 and directed back to the computing device 650. The same process is repeated for known tissue, and optionally other known targets such as surgical components and/or surgical treatment area medium (e.g., liquid).

If tissue is the known target material, then at 1006 the distal end of the scope is moved over the known target tissue and data (reflected intensity signal data) is collected (e.g., by having the surgeon depress a foot pedal or an assistant presses a button on the screen) for a predetermined period of time (e.g., 20 seconds) while the treatment laser 610 is off. This entails light from light source 615 being directed to the target tissue and the reflected light from this source off the target area being directed to detection channels as described herein.

During calibration, the treatment laser power can be disabled or lowered to a safe level for soft tissue, and for stone material, the treatment laser can be configured to emit power above the ablation threshold for the stone to guarantee contact or quasi-contact between the fiber tip and stone (typically 0-1.5 mm). For instance, if stone is the known target material, then at 1008 the distal end of the scope is moved over the target stone for a predetermined period of time (e.g., 1-20 seconds) and data is collected (e.g., by having the surgeon depress a foot pedal) while the treatment laser 610 is on, but at a low pulse energy setting. For instance, the pulse energy may be less than 0.5 J, with some examples having a pulse energy of 0.025 to 0.1 J, with a 10-100 Hz repetition rate and an average power of 1 to 10 watts (W). In one embodiment, the pulse energy is sufficient to create some dusting as the operator moves the fiber across the stone surface. According to another example, the pulse energy is about 0.1 J with a peak power at about 500 W, with a 60 Hz repetition rate. In this example the settings ensure that the calibration signal is obtained from the bulk of the stone, and not only from a thin superficial layer that may not be representative of the actual stone. Reflected light from light source 615 during this action is detected by the detection channels.

At 1010 the process 100 can include a computing device (feedback analyzer 650) analyzing data received from the detection channels. This includes utilizing one or more “baseline” raw reflected intensity signals and calculated ratios of reflected light intensity within selected bands of wavelengths from each type of material that is stored in a database of the computing device.

As mentioned previously, a ratiometric technique can be applied to the reflected intensity data obtained from the known targets (and targets (that may be unknown or otherwise not verified) during the procedure, as explained in further detail below). At least one ratio of a reflected light intensity of one selected wavelength band to a reflected light intensity of a different selected wavelength band is generated or otherwise calculated by computing device 650 at step 1012. As an example, in some embodiments three different photodetectors are used (i.e., three channels) which yields a reflected intensity values for each wavelength band: wavelength band 1 (I₁), wavelength band 2 (I₂), and wavelength band 3 (I₃). At least three different ratios can then be calculated: R₁=I₁/I₂, R₂=I₂/I₃, and R₃=I₁/I₃. In one embodiment, the different selected wavelength bands are selected from a group consisting of: about 400-410 nm, about 440-480 nm, about 460-480 nm, about 510-530 nm, about 540-560 nm, about 550-570 nm, about 570-580 nm, about 580-600 nm, about 600-620 nm, about 690-710 nm, about 740-760 nm, about 790-810 nm, about 920-940 nm, about 970-990 nm, and about 1150-1350 nm. In this example, two materials are of interest, stone and tissue (with stone being the eventual desired target for the treatment laser). Many samples can be taken for each of the “known” tissue and stone materials. In some embodiments, up to five different photodetectors are used (i.e., five channels).

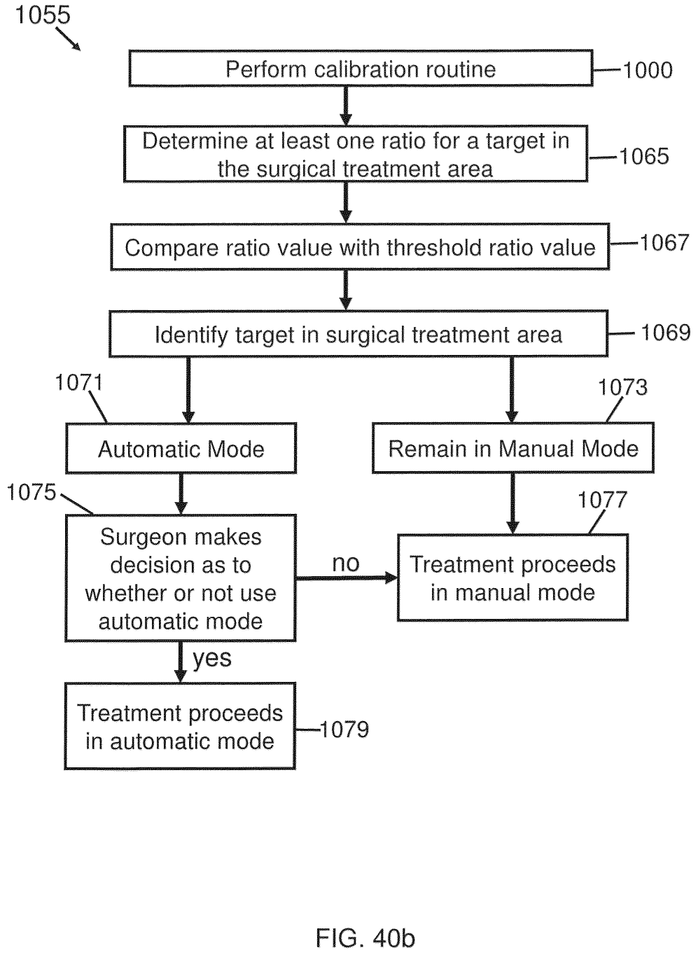

At step 1014 at least one histogram representation of values from each ratio (of at least one ratio) is then generated or otherwise determined by computing device 650 (for each (known) target). Two examples of histograms or plot of the distribution of ratio values are shown in FIGS. 41a and 41b, where a value of the number of samples (x-axis) is plotted against one ratio value (e.g., R₁, R₂, or R₃) (y-axis) for each of the stone and tissue materials.

FIGS. 41a and 41b are histograms (for a distribution of ratios of reflected signal intensity collected during calibration) of two different examples of stone and soft tissue distinction, where FIG. 41a shows an example of a “good” separation (where the reflected intensity ratios indicate the target is the desired target e.g., a stone and not tissue), and FIG. 41b shows an example of a “bad” separation (where the reflected intensity ratios indicate that either the target is not the desired target (tissue, when the desired target is stone) or it is not clear that the desired target is one or the other). The reflected intensity ratio values can also be used to detect if the object in front of the distal end of the tip of the scope is an instrument (e.g., a surgical component such as a catheter, a basket, a stent, a sheath, etc.). In this instance, a calibration routine would involve having the physician obtain reflected intensity signal values from a “known” catheter, basket, stent, or sheath via the same procedure as outlined above via the FOV from the camera.

In at least one embodiment, and in continuation of the example using three different ratios as discussed above, three different histograms may be generated, one for ratio R₁, one for ratio R₂, and one for ratio R₃. FIG. 41a may represent a non-limiting example of the histogram results from R₁, and FIG. 41b may represent a non-limiting example of the histogram results from R₃. Clear (i.e., “good”) separation between the tissue and stone materials is shown in FIG. 41a and “bad” separation between the tissue and stone materials is shown in FIG. 41b. The analysis discussed below would result in the ability for the data (and ratio) associated with FIG. 41a to provide a framework for the actual stone ablation procedure that is performed after the calibration.

In its simplest form, the in-patient calibration establishes differentiation between soft tissue and calculi (e.g., see FIGS. 41a and 41b). During the calibration process, the reflected signals are collected in all N wavelength channels. Then N (N−1)/2 unique pairs of the signals are formed, the respective signal ratios are computed, and each pair is analyzed in terms of quality of stone-tissue separation. As previously outlined, FIG. 41a may be the result from R₁ (using the example from above), and FIG. 41b may be the result from R₃.

Various specific techniques can be used to analyze the signal ratio(s) to optimize stone and tissue separation (distinction). In one embodiment, the concept of a ratio value associated with a predetermined percentile for each known target (based on the respective histograms) is used. This is shown as step 1016 in FIG. 40a. In this example, the computing device 650 determines a ratio value associated with a predetermined percentile for each known target based on the histogram. For example, two ratio values corresponding or otherwise associated with an 80^{th} percentile (the predetermined percentile in this example) of all tissue and stone samples, S_{T80} and S_{S80} respectively, are computed. For example, the line marked “Min” in FIG. 41a may mark S_{S80} (the R₁ ratio value associated with the 80^{th} percentile for stone) where 80% of the stone histogram data falls to the left of S_{S80} and 20% of the stone histogram data falls to the right of S_{S80}. The line marked “Max” in FIG. 41a may mark S_{T80} (the R₁ ratio value associated with the 80^{th} percentile for tissue) where 80% of the tissue histogram data falls to the right of S_{T80} and 20% of the tissue histogram data falls to the left of S_{T80}. In a similar manner, the line marked “Min” in FIG. 41b may mark S_{S80} (the R₃ ratio value associated with the 80^{th} percentile for stone) and the line marked “Max” in FIG. 41b may mark S_{T80} (the R₃ ratio value associated with the 80^{th} percentile for tissue).

In one embodiment, a difference value between the ratio values associated with the predetermined percentiles for each known target is accepted as the quality of separation. This is shown as step 1018 in FIG. 40a. This step may include determining a difference value between a first ratio value associated with the predetermined percentile for a first known target (of at least two known targets) and a second ratio value associated with the predetermined percentile for a second known target. The wavelength pair with the best separation (e.g., the ratio value R associated with the largest difference value _{R}, as explained in further detail below) is then selected for subsequent use during the procedure. For example, the difference value between S_{S80} and S_{T80} for R_{1 R1} in FIG. 41a and the difference value between S_{S80} and S_{T80} for R_{3 R3} in FIG. 41b.

It should be noted that in the flowchart of FIG. 40a, the analysis output of step 1012 may employ a form other than a histogram (step 1014) as the output. For instance, the analysis may produce graphical or other types of data representations and models (e.g., pie charts, bar charts, data matrix), or any other output capable of functioning as a basis for the determining the ratio value associated with the predetermined percentile for each known target.

In at least one embodiment, the difference value (associated with each ratio value) is compared to a threshold difference value. In one embodiment, the threshold difference value is _{R1} (a first difference value) is _{R3} (a second difference value) and computing device 650 determines whether the first difference value or the second difference value is larger. In response to a determination that the first difference value is larger than the second difference value, then the first difference value is selected as the threshold difference value, and in response to a determination that the second difference value is larger than the first difference value, selecting the second difference value as the threshold difference value. In the above exampl _{R1} is larger _{R3}, signifying a greater degree of separation between stone and tissue material distinction, _{R1} may be chosen or otherwise be used as the threshold difference value. In other embodiments, a threshold difference value may be established by an operator, or may be established by the computing device 650 (e.g., on the basis of stored data, other stored information, mathematical algorithm(s), etc.).

Once the reflected intensity wavelength pair is defined (i.e., which ratio R value yields the best separation criteria), the actual separation criterion S_{SP} may also be defined in various ways. In one embodiment, S_{SP} can be computed as an average of S_{T80} and S_{S80}, which is explained in further detail below.

In accordance with one embodiment, and using the framework set out in the example from above, in response to a determination that the difference value meets or exceeds the threshold difference value, a threshold ratio value (or range of values) can be established that is based at least in part on the first ratio value and the second ratio value (e.g., establishing a threshold ratio value based at least in part on the multiple reflected light intensity values from each known target). This can be performed by the computing device 650 and is shown as step 1022 in FIG. 40a. For instance, using the example from above, FIG. 41a (associated with R₁) _{R1} that meets or exceeds the threshold difference value. The first ratio value associated with the predetermined percentile for the first known target (stone) is approximately 0.57 in FIG. 41a. The second ratio value associated with the predetermined percentile for the second known target (tissue) is approximately 0.66. A threshold ratio value may be established or otherwise determined based on the first ratio value and the second ratio value. For example, an average ratio value based on the first and second ratios may be established as the threshold ratio value. Referring to FIG. 41a, the line marked “Medium” line as shown in FIG. 41a marks an average (i.e., 50%) between the 0.57 value of the first ratio and the 0.66 value of the second ratio. This line can be established as the threshold ratio value (having a value of approximately 0.61 for this example in FIG. 41a). During an actual procedure, and in accordance with at least one embodiment, a comparison of a ratio obtained during a “live” surgical treatment procedure can be compared against the threshold ratio value. A target (in the “live” surgical treatment area during the course of an actual procedure) can be associated with a known target based on this comparison. For example, using FIG. 41a, R₁ values associated with a target in an actual surgical treatment area during a procedure can be compared to the threshold ratio value of 0.61. Ratio values greater than 0.61 (to the right of the 50% line) will associate the target in the actual surgical treatment area with tissue material and ratio values less than 0.61 (to the left of the 50% line) will associate the target in the actual surgical treatment area with stone material.

In a different embodiment, the computing device 650 is configured to establish each ratio associated with the predetermined percentile as a threshold ratio value for the respective known material. The threshold ratio value can be used during an actual procedure to verify that the object in front of the camera is the actual desired target. For instance, using the example from _{R1} meets or exceeds the threshold difference value, and the ratio values associated with the 80^{th} percentiles for each of tissue and stone can be used during an actual procedure. For stone, the threshold ratio value for R₁ is approximately 0.57 and for tissue the threshold ratio value for R₁ value is approximately 0.66 in the example shown in FIG. 40a. During an actual procedure, if the ratio value for R₁ obtained from “live” measured reflected intensity data (from a target in the surgical treatment area) meets or exceeds the predetermined percentile associated with the corresponding threshold ratio value, then the target can be associated with the known target. For example, if the R₁ value from the “live” procedure is calculated as 0.55, then this value meets or exceeds the 80^{th} percentile (predetermined percentile) associated with the corresponding threshold ratio value of 0.57 for R₁ of the known stone material and the target material can be associated with a known target (in this case stone). If the R₁ value from the “live” procedure is calculated as 0.67, then this value meets or exceeds the 80^{th} percentile associated with the corresponding threshold ratio value of 0.66 for R₁ of the known tissue material and the target material can be associated with a known target (in this case tissue). Note that in this example the R₁ value of 0.66 would not meet or exceed the predetermined percentile value associated with the corresponding threshold ratio value for R₁ of the stone material and therefore the target in this second instance cannot be identified as stone.

It is to be appreciated that other selection criterion may perform the functionality of the threshold ratio value. For example, other math formulas or algorithms may be used to establish a source of comparison or setting reference. Furthermore, in some embodiments the selection criteria may be automatically and dynamically adjusted during the procedure, based on the log (store data) of recorded stone/non-stone signals and operator's actions.

It is noted that during calibration if no pair (of ratio values) provides sufficient separation between the targets, then automatic control of laser emission may be disabled or, alternatively calibration may be attempted again with different laser settings (e.g., laser power).

### During Procedure

In accordance with one embodiment, the analysis performed in the calibration forms the basis for the actual treatment procedure. A flowchart for one example of a lithotripsy procedure or process 1055 is shown in FIG. 40b in accordance with one embodiment. Broadly speaking, the analysis performed in the calibration (step 1000 in FIG. 40b, an example of which is shown in FIG. 40a) forms the basis for the actual lithotripsy procedure, including the basis for whether an automatic mode 1071 is initiated (or recommended to a surgeon), or the system recommends remaining in manual mode at 1073 during the actual procedure.

According to at least one embodiment, the computing device 650 is configured to generate a control signal for controlling operation of the treatment laser 610 based on the identification of the target 630. In some embodiments, the target is a stone, and the non-treatment target is tissue or a surgical component or a surgical treatment area medium (e.g., liquid). In some embodiments, the control signal includes activation, de-activation, or an operating parameter setting for the treatment laser. Non-limiting examples of operating parameter settings for the treatment laser include pulse peak power, pulse shape, pulse width, pulse energy, interval between pulses, repetition rate, average power, and continuous wave (CW) power.

The reflected signal intensities may also be used during the course of a procedure. As mentioned previously, at least one ratio for a target in the surgical treatment area can be determined by computing device 650 from the reflected intensity value(s) using the photodetectors 646a-646i. This is shown as step 1065 in FIG. 40b. For example, if the calibration routine produced results that indicated that R₁ provided the best difference value (and hence the best separation), then R₁ would be determined for an actual target in the surgical treatment area. The ratio value R₁ of an actual target would then be compared (e.g., by the computing device 650) with the threshold ratio value that was determined in the calibration routine (as indicated at step 1067 in FIG. 40b). The computing device 650 then associates the target in the surgical treatment area with a known target based on the comparison. At step 1069 the target in the surgical treatment area is identified. For example, in response to a determination that the known target is a treatment target (e.g., stone), the target is identified as a treatment target, and in response to a determination that the known target is not a treatment target, the target is identified as a non-treatment target (e.g., tissue, surgical component, treatment medium). The type of treatment target is input to the computing device 650 by an operator.

Once the target in the surgical treatment area is identified, then either an automatic mode (step 1071) or a manual mode (1073) is recommended to the surgeon (or operator). For example, if the target in the surgical treatment area is identified as stone material, then an automatic mode may be recommended by the computing device 650. This recommendation can be relayed to the surgeon any one of a number of different ways, e.g., through a visual representation (on a screen), and/or audible notification, and/or tactile notification, as described in more detail below.

If automatic mode is recommended, then at 1075 the surgeon makes a decision as to whether or not the treatment proceeds (yes) in automatic mode (at 1079) or (no) proceeds in manual mode (at 1077). Automatic mode enables the computing device 650 to control the firing of the laser (laser emission) without the aid of the surgeon. Manuel mode allows for the surgeon to remain in control of actuating the laser device 610 to fire.

In accordance with at least one embodiment, the procedure terminates once all stones above a certain size have been removed (e.g., by ablation) from the surgical treatment area (e.g., ureter, urethra, kidney, bladder, etc.). In some embodiments, this entails stone particles that are less than 250 microns in diameter, since these can be passed naturally by the human body. In some embodiments, the procedure removes stones with an efficacy such that no stones are detectable in the patient at a 7-day post-surgery follow-up appointment.

As will be recognized by those of skill in the art, conventional lithotripsy procedures make use of a foot switch or foot pedal that is activated by the surgeon or user that in turn functions to activate the treatment laser and hence stone fragmentation. Conventionally, the foot switch is activated under direct visual control by the user. This action can be performed many times during the procedure because the user will switch off the laser when he or she sees that the fiber tip is in proximity to the mucosal wall and determines that damage to the mucosal wall may occur if the laser fires. Repeated on/off cycling of the footswitch leads to a longer procedure time, and fatigue in the user creates the potential to direct laser energy at an unintentional target, such as tissue. In accordance with an additional aspect of this disclosure, the user may continuously depress the foot pedal and the computing device 650 (when in an automatic mode of operation) actually activates the laser and modulates the output based on the identified target. For example, if the treatment laser is positioned in front of identified stone material (when the treatment target is stone), the computing device 650 will control the treatment laser to fire the laser, and if a few seconds later the treatment laser is positioned in front of tissue, (a non-treatment target) the computing device 650 will control the treatment laser to de-activate, all while the foot pedal is still depressed by the user. This reduces procedure time, eliminates fatigue by the user, and allows for a safer procedure.

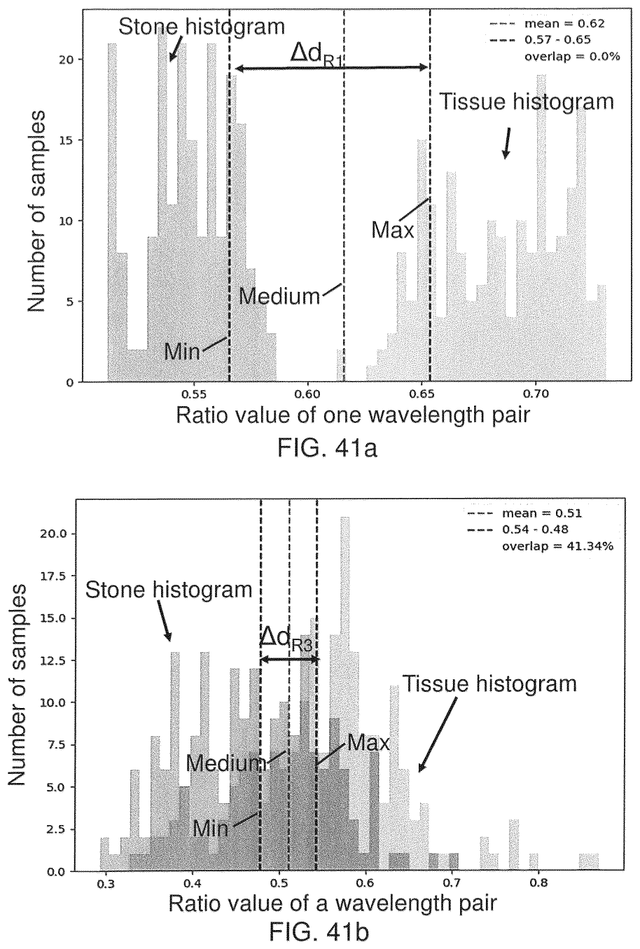

According to another embodiment, the operator can have an opportunity to use and/or adjust the separation criterion during the actual procedure to optimize the efficacy/safety balance. In accordance with at least one embodiment, determining whether the target is a treatment target or a non-treatment target is performed in between every N laser pulses emitted by a treatment laser. In one embodiment, feedback signals (reflected light intensities of light 615 reflected off target) are analyzed during the procedure between the laser pulses, e.g., after every Nth pulse, and in some embodiments, N is 1 so the analysis occurs after every pulse. In some embodiments, determining whether the known target is a treatment target or a non-treatment target is performed after modifying a laser operating parameter of a treatment laser. For instance, target analysis may be performed if the treatment laser power, frequency, or other operating parameter is modified or otherwise changed. An example of this process is shown in the time schematic at the bottom of FIG. 42. In this example, in between each pulse, the reflected signal ratios are analyzed to determine if the distal end of the scope (which includes the laser fiber) is positioned in front of a stone, tissue, or instrument (surgical component). The desired target in this instance is stone material and laser emission is disabled when stone material is not detected (to prevent tissue damage) or enabled or otherwise resumed when stone material is detected.

In accordance with various aspects, the system is configured to operate in either a “passive” or “active” mode. In the passive mode, the system will notify the operator when the fiber is not positioned in front of or on the stone (based on the selected separation criterion). The notification to the operator can be achieved through a variety of methods, including (but not limited to), audio (e.g., a specific tone or voice message), visual (e.g., through light indicators), tactile (e.g., through vibrating mechanical feedback, such as in the scope handle), or picture-in-picture video (e.g., when information from the feedback sensor is added to the scope video to create an augmented picture). In the active mode, the system (i.e., computer controller/computing device) will have control over the laser emission.

Emergency Room application—In accordance with certain embodiments, a laser system configured in a similar manner as described herein in reference to system 600 can be implemented in an emergency room environment. For example, the identification of the target as a treatment target or a non-treatment target based at least in part on the optical data and the predetermined calibration based on at least two known targets may be performed during an emergency room visit by a patient. In such a setting, a patient arrives at the emergency room with a stone positioned in the urethra. The endoscope can be positioned by emergency room personnel in the urethra with the fiber tip positioned in front of the stone. This can be performed by the user using the imaging camera incorporated into the endoscope and coupled with a video screen. The calibration routine (as outlined in FIG. 40a) can be performed by the computing device 650 in combination with the user directing the distal tip of the endoscope to known targets including the stone material and tissue material (e.g., wall of urethra). An aspect of this method and implementation is that the user (e.g., emergency room surgeon) can perform the procedure without comprehensive urological training and thus substantially increase the quality of care for the patient and simultaneously reduce the cost of the procedure.

### Other Calibration Options

Returning now to aspects related to the calibration procedure outlined in FIG. 40a, according to some embodiments the in-patient calibration procedure can be configured with additional capabilities or functionalities. For example, in one embodiment an additional calibration can be performed in the ambient medium (liquid), i.e., when all the targets are sufficiently (e.g., more than 3 to 7 mm) far away from the distal tip of the fiber. Such information can help improve recognition of the situation when the fiber is too far from the target to deliver the energy in an efficient manner. On the other hand, in this situation the system can allow laser emission if the “popcorn” mode of operation is selected by the operator. In addition, an extra calibration procedure may be performed with the tools/instruments placed in or near the procedure fields (e.g., stent, ureteral access sheath etc.), as mentioned previously (i.e., the stent, ureteral access sheath, and other surgical components would be “known” targets in the calibration routine).

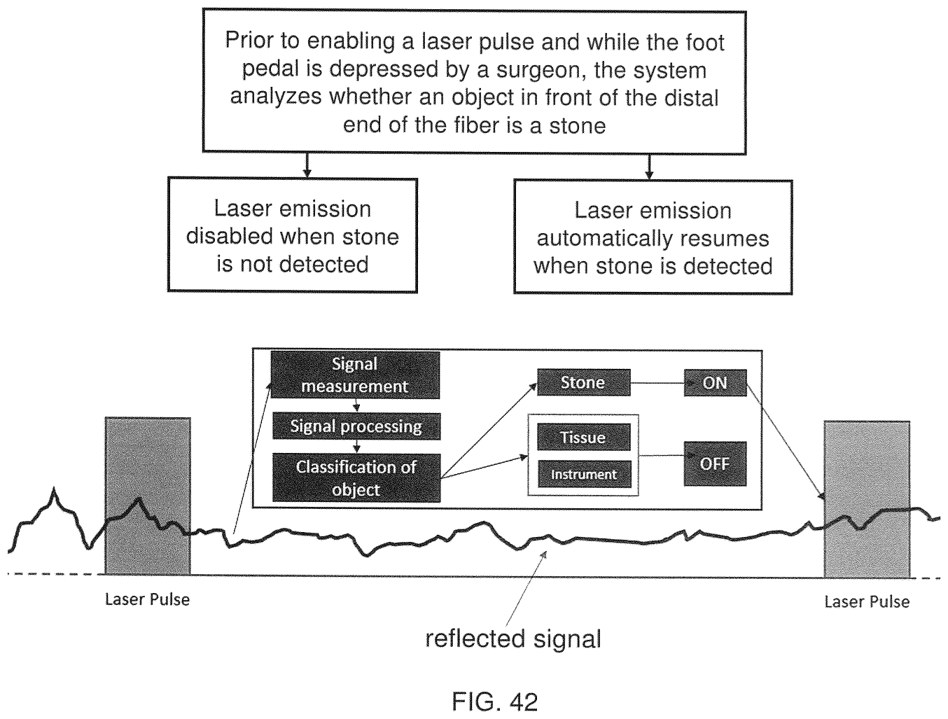

In accordance with an additional embodiment, the use of a decision space can be implemented for purposes of evaluating reflected intensity signals. This approach can function to enhance the quality of the stone/tissue separation evaluation. In particular, rather than selecting one single pair of the wavelengths to proceed, two or more pairs demonstrating the best results can be used. A multidimensional decision space having n decision options based on at least two ratios may be defined, where n is the number of known targets. FIG. 43 is a schematic of one example of a two-dimensional decision space using two pairs of selected wavelengths (i.e., two ratios of reflected intensity values, e.g., R₁ (I₁/I₂) and R₂ (I₂/I₃). In this example, n=3 (since the number of known targets is 3: stone, tissue, and ambient medium (in the surgical treatment environment)), and the decision space becomes two-dimensional (2D). In some embodiments, multidimensional threshold separation lines between the n decision options (separation surfaces) can be defined in the multidimensional decision space for discrimination between each target of the known targets. This approach is also shown in FIG. 43, where a single separation criterion becomes a separation line, as indicated. When the number of the wavelength pairs used further increases, the dimensionality of the decision space increases as well. For example, three wavelength pairs would result in a 3D space and separation surfaces.

As previously discussed, the robustness of stone/tissue separation can be further enhanced by using two or more pairs (of wavelengths) (i.e., ratios). In the situation where three wavelengths are selected, at least three ratios (of pairs) are available. In most cases, two pairs provide good separation between the stones and soft tissue while the third pair shows minimal separation. Use of the two “best separation” pairs can provide a more accurate distinction or discrimination between the targets than use of just one pair.

According to at least one embodiment, a weighting factor may be assigned to a ratio associated with the largest difference value. For instance, if two ratios R₁ and R₂ are calculated, then the ratio R that provides better separation _{R}) is assigned a greater weightage. For weight, a quantity called “effect size” commonly used in statistical methods is used. The effect size takes into account both the difference in means as well as variability with the multiple measurements for the two different targets.

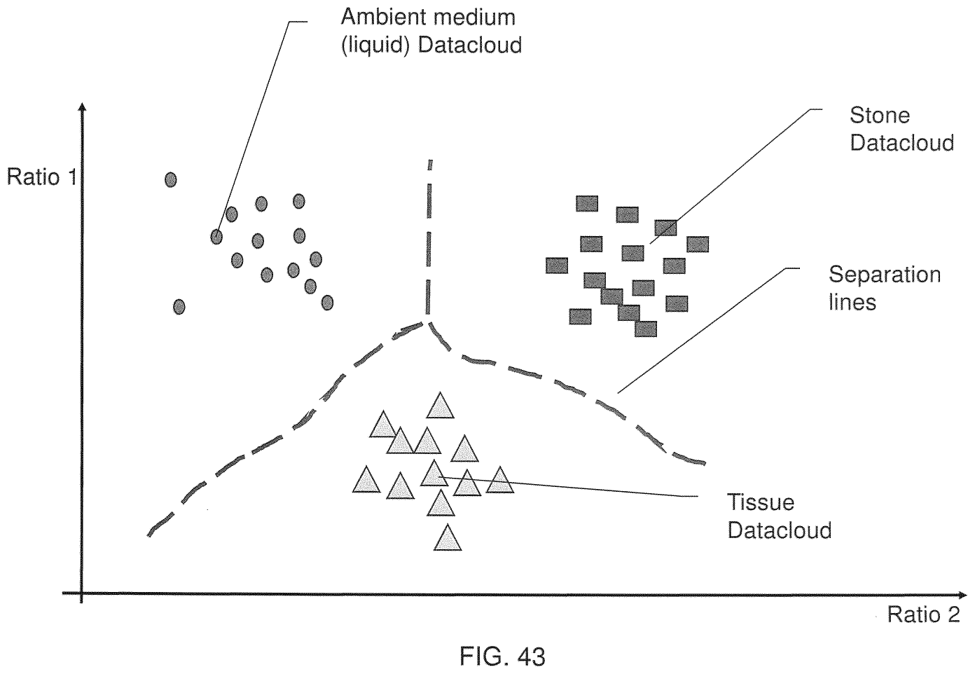

Method 1: According to one embodiment, a linear combination of the two ratios with the weighting factors is used. An example of this approach is shown in FIG. 44, which shows histograms collected during calibration for two ratios, with ratio 1, r1 on the left side, and ratio 2, r2 shown on the right side of FIG. 44. The weighted ratio is calculated as:
(alpha1*r1+alpha2*r2)/(alpha1+alpha2) where alpha1, alpha2 are the weighting factors and calculated from

$$\\ alpha 1 = effect\_size \_ 1 = ( r 1 \_m \_soft - r 1 \_m \_stone ) / ( geometric mean of s 1 \_stone and s 1 \_soft ) ,$$

$$\\ alpha 2 = effect\_size \_ 2 = ( r 2 \_m \_soft - r 2 \_m \_stone ) / ( geometric mean of s 2 \_stone and s 2 \_soft )$$

where
r1=ratio 1
r2=ratio 2
r1_m_stone=mean of r1 for stone
r1_m_soft=mean of r1 for tissue
r2_m_stone=mean of r2 for stone
r3_m_soft=mean of r2 for tissue
s1_stone=standard deviation for ratio 1 of stone
s1_soft=standard deviation for ratio 1 of tissue
s2_stone=standard deviation for ratio 2 of stone
s2_soft=standard deviation for ratio 2 of tissue

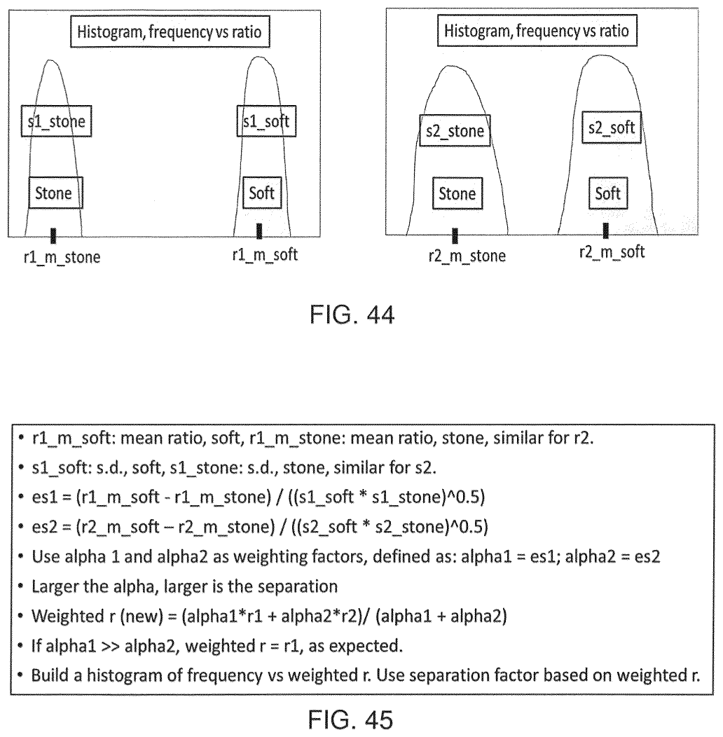

FIG. 45 is a schematic explaining the variables used, the calculation of the effect size, the weighting factors, and the build of histogram post calibration measurements. In another embodiment, in computation of the effect size, instead of the mean, other measures of the center of the data are used rather than the mean, such as the median.

Method 2: According to another embodiments, optimization by varying the weighting factors may be used. For example, weighting factors can be defined as beta1 and beta2 so the expression for weighted r is

$$\\ Weighted r = ( beta 1 * r 1 + beta 2 * r 2 ) / ( beta 1 + beta 2 )$$

For beta1: lower-limit: upper-limit: increment
   For beta2: lower-limit: upper-limit: increment
      Calculate weighted r.
   End loop for beta2
End loop for beta1

In accordance with at least one aspect the (beta1, beta2) combination that maximizes the distinction is determined. As an example, distinction=ratio of effect-sizes (where effect-size is the ratio of mean/size distribution (s.d.) for stone, soft tissue).

In some embodiments, information/data from prior procedures may be used as part of the analysis. In accordance with an additional embodiment, the target may be identified as a treatment target or a non-treatment target based at least in part on a comparison against stored data from previously recorded reflected intensity values. It is noted that stored data refers to data that is not indicative of the current target site and can refer to reflected intensity values previously measured and recorded or otherwise collected from prior procedures or experiments and stored in a database (prior to the procedure being performed). The stored data may correspond to a multivariate dataset according to some embodiments. The computing device 650 may be configured to make this comparison. In some embodiments, information about already performed clinical cases can be organized and stored into a database. The database can be structured in terms of anatomical locations (bladder, ureter, kidney), the instrument used (rigid, flexible), fiber size, etc. Once these parameters are defined for a planned procedure, the data points from the database can be added (and in some instances with a certain weight) to the in-patient calibration data points.

In accordance with other embodiments, the target may be identified as a treatment target or a non-treatment target based at least in part on a mathematical model and/or algorithm that is utilized by the computing device 650.

According to some embodiments, the target is identified as a treatment target or a non-treatment target based at least in part on a machine learning model. The computing device 650 may be configured to utilize the machine learning model (and/or execute a machine learning model) for making this assessment. For example, an artificial intelligence system can analyze the success rate of the previously performed procedures and adjust the separation criteria (lines, surfaces etc.) using machine-learning techniques. The machine learning algorithm may be implemented via machine learning software installed on the computing device 650 and typically comprises a neural network and is configured to undergo at least one training phase as understood by those skilled in the art.

Use of the smart sensor (e.g., in system 600) can measurably improve clinical outcomes of the lithotripsy procedure. The list of parameters (not all-inclusive) that can be improved include:
Number of mucosal thermal damage zones
Total surface area of mucosa thermal damage
Total procedure time
Total laser procedure time (i.e., the time duration from the time the laser is first turned on until the laser is last turned off)
Total footswitch (foot pedal) ON time
Total laser emission time (i.e., the total time the laser is firing)
Actual duty cycle=total emission time/total laser ON time
Number of footswitch press/release cycles
Stone treatment time (ablation rate)
Stone-free rate
Fragments size distribution
Complications grading

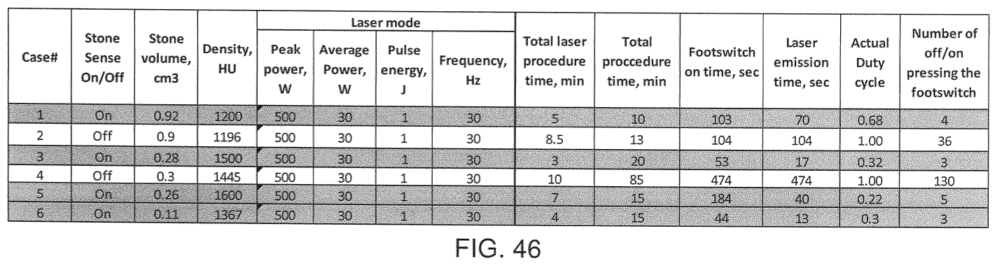

FIG. 46 is a table listing six (6) examples of lithotripsy procedures performed on actual patients. Cases 1, 3, 5, and 6 show results from procedures performed using a system as described above in reference to system 600 of FIG. 39 and demonstrate the beneficial effects of using the smart sensor system. For example, in all cases with active stone identification, the total laser procedure as well as the total procedure time were reduced in comparison to respective comparison cases 2 and 4, which were performed without the use of the smart sensor configuration. In addition, laser emission time, the actual duty cycle, and the number of off/on foot pedal cycles were reduced in the smart sensor cases when compared to their control cases. The amount of time the foot pedal was on was also reduced for the smart sensor cases.

In accordance with an additional aspect, the system can be used to recognize bleeding and suggest to the doctor use of hemostatic parameters. In the active mode, switch to the hemostatic parameters can be automated.

The present disclosure has described one or more preferred non-limiting examples, and it should be appreciated that many equivalents, alternatives, variations, and modifications, aside from those expressly stated, are possible and within the scope of the disclosure.

It is to be understood that the disclosure is not limited in its application to the details of construction and the arrangement of components set forth in the following description or illustrated in the following drawings. The disclosure is capable of other non-limiting examples and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

As used herein, unless otherwise limited or defined, discussion of particular directions is provided by example only, with regard to particular non-limiting examples or relevant illustrations. For example, discussion of “top,” “front,” or “back” features is generally intended as a description only of the orientation of such features relative to a reference frame of a particular example or illustration. Correspondingly, for example, a “top” feature may sometimes be disposed below a “bottom” feature (and so on), in some arrangements or non-limiting examples. Further, references to particular rotational or other movements (e.g., counterclockwise rotation) is generally intended as a description only of movement relative a reference frame of a particular example of illustration.

In some non-limiting examples, aspects of the disclosure, including computerized implementations of methods according to the disclosure, can be implemented as a system, method, apparatus, or article of manufacture using standard programming or engineering techniques to produce software, firmware, hardware, or any combination thereof to control a processor device (e.g., a serial or parallel general purpose or specialized processor chip, a single- or multi-core chip, a microprocessor, a field programmable gate array, any variety of combinations of a control unit, arithmetic logic unit, and processor register, and so on), a computer (e.g., a processor device operatively coupled to a memory), or another electronically operated controller to implement aspects detailed herein. Accordingly, for example, non-limiting examples of the disclosure can be implemented as a set of instructions, tangibly embodied on a non-transitory computer-readable media, such that a processor device can implement the instructions based upon reading the instructions from the computer-readable media. Some non-limiting examples of the disclosure can include (or utilize) a control device such as an automation device, a special purpose or general purpose computer including various computer hardware, software, firmware, and so on, consistent with the discussion below. As specific examples, a control device can include a processor, a microcontroller, a field-programmable gate array, a programmable logic controller, logic gates etc., and other typical components that are known in the art for implementation of appropriate functionality (e.g., memory, communication systems, power sources, user interfaces and other inputs, etc.).

The term “article of manufacture” as used herein is intended to encompass a computer program accessible from any computer-readable device, carrier (e.g., non-transitory signals), or media (e.g., non-transitory media). For example, computer-readable media can include but are not limited to magnetic storage devices (e.g., hard disk, floppy disk, magnetic strips, and so on), optical disks (e.g., compact disk (CD), digital versatile disk (DVD), and so on), smart cards, and flash memory devices (e.g., card, stick, and so on). Additionally it should be appreciated that a carrier wave can be employed to carry computer-readable electronic data such as those used in transmitting and receiving electronic mail or in accessing a network such as the Internet or a local area network (LAN). Those skilled in the art will recognize that many modifications may be made to these configurations without departing from the scope or spirit of the claimed subject matter.

Certain operations of methods according to the disclosure, or of systems executing those methods, may be represented schematically in the FIGS. or otherwise discussed herein. Unless otherwise specified or limited, representation in the FIGS. of particular operations in particular spatial order may not necessarily require those operations to be executed in a particular sequence corresponding to the particular spatial order. Correspondingly, certain operations represented in the FIGS., or otherwise disclosed herein, can be executed in different orders than are expressly illustrated or described, as appropriate for particular non-limiting examples of the disclosure. Further, in some non-limiting examples, certain operations can be executed in parallel, including by dedicated parallel processing devices, or separate computing devices configured to interoperate as part of a large system.

As used herein in the context of computer implementation, unless otherwise specified or limited, the terms “component,” “system,” “module,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as non-limiting examples of the disclosure, of the utilized features and implemented capabilities of such device or system.

As used herein, unless otherwise defined or limited, ordinal numbers are used herein for convenience of reference based generally on the order in which particular components are presented for the relevant part of the disclosure. In this regard, for example, designations such as “first,” “second,” etc., generally indicate only the order in which the relevant component is introduced for discussion and generally do not indicate or require a particular spatial arrangement, functional or structural primacy or order.

As used herein, unless otherwise defined or limited, directional terms are used for convenience of reference for discussion of particular figures or examples. For example, references to downward (or other) directions or top (or other) positions may be used to discuss aspects of a particular example or figure, but do not necessarily require similar orientation or geometry in all installations or configurations.

This discussion is presented to enable a person skilled in the art to make and use non-limiting examples of the disclosure. Various modifications to the illustrated examples will be readily apparent to those skilled in the art, and the generic principles herein can be applied to other examples and applications without departing from the principles disclosed herein. Thus, non-limiting examples of the disclosure are not intended to be limited to non-limiting examples shown, but are to be accorded the widest scope consistent with the principles and features disclosed herein and the claims below. The following detailed description is to be read with reference to the figures, in which like elements in different figures have like reference numerals. The figures, which are not necessarily to scale, depict selected examples and are not intended to limit the scope of the disclosure. Skilled artisans will recognize the examples provided herein have many useful alternatives and fall within the scope of the disclosure.

The aspects disclosed herein in accordance with the present disclosure, are not limited in their application to the details of construction and the arrangement of components set forth in the following description or illustrated in the accompanying drawings. These aspects are capable of assuming other non-limiting examples and of being practiced or of being carried out in various ways. Examples of specific implementations are provided herein for illustrative purposes only and are not intended to be limiting. In particular, acts, components, elements, and features discussed in connection with any one or more non-limiting examples are not intended to be excluded from a similar role in any other non-limiting examples.

Also, the phraseology and terminology used herein is for the purpose of description and should not be regarded as limiting. Any references to examples, non-limiting examples, components, elements or acts of the systems and methods herein referred to in the singular may also embrace non-limiting examples including a plurality, and any references in plural to any non-limiting example, component, element or act herein may also embrace non-limiting examples including only a singularity. References in the singular or plural form are not intended to limit the presently disclosed systems or methods, their components, acts, or elements. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms. In addition, in the event of inconsistent usages of terms between this document and documents incorporated herein by reference, the term usage in the incorporated reference is supplementary to that of this document; for irreconcilable inconsistencies, the term usage in this document controls. Moreover, titles or subtitles may be used in the specification for the convenience of a reader, which shall have no influence on the scope of the present disclosure.

Having thus described several aspects of at least one example, it is to be appreciated that various alterations, modifications, and improvements will readily occur to those skilled in the art. For instance, examples disclosed herein may also be used in other contexts. Such alterations, modifications, and improvements are intended to be part of this disclosure, and are intended to be within the scope of the examples discussed herein. Accordingly, the foregoing description and drawings are by way of example only.

As used herein, “relevant quantity”-scalar or vector quantity obtained from the collected data through application of at least one of the following operations: weighted integration, weighted differentiation, averaging, normalization to reference data, addition, subtraction, multiplication, division. The particular set and order of operations is selected by the final analysis objective (e.g., discrimination of target vs. non-target tissue). So obtained quantity is then compared to a set of generally multi-dimensional thresholds to achieve desired analysis goal.

As used herein, “data” or “optical data”-any sequence or combination of signals obtained from the optical detectors in the system, including, but not limited to, raw signal values, temporal profiles of the signals, spectral signatures of the signals, max/min values of the signals, correlation functions of the signals, mean values of the signals over a time period, standard variations of the signals.

As used herein, target material and stone can be used interchangeably. For example, a target material can be a stone, and a stone can be a target material.

It is to be appreciated that although most of the non-limiting examples disclosed herein deal with laser lithotripsy of urinary calculi, other applications that address other conditions, such as bladder and other body stones, tissue incision, vaporization, and coagulation, other body areas and forms of directed energy are also within the scope of this disclosure.

Various features and advantages of the disclosure are set forth in the following claims.

What is claimed is:

1. A method for controlling a surgical laser system comprising:
providing a surgical fiber configured to receive light reflected from a target in a surgical treatment area; and
providing a computing device configured to couple with at least two photodetectors, each photodetector configured to detect an intensity of reflected light from the target in a different selected wavelength band, the computing device further configured to:
receive the reflected light intensity in at least two selected wavelength bands;
generate optical data corresponding to the reflected light intensity; and
identify the target as a treatment target or a non-treatment target based at least in part on the optical data and a predetermined calibration based on at least two known targets.

2. The method of claim 1, further comprising performing the predetermined calibration.

3. The method of claim 2, wherein at least one of generating the optical data and performing the calibration includes determining at least one ratio of a reflected light intensity of one selected wavelength band to a reflected light intensity of a different selected wavelength band.

4. The method of claim 3, wherein performing the predetermined calibration further comprises:
obtaining multiple reflected light intensity values from each known target of the at least two known targets; and
establishing a threshold ratio value based at least in part on the multiple reflected light intensity values from each known target.

5. The method of claim 4, further comprising:
determining a ratio value associated with a predetermined percentile for each known target based on the multiple reflected light intensity values from each known target;
determining a difference value between a first ratio value associated with the predetermined percentile for a first known target and a second ratio value associated with the predetermined percentile for a second known target;
comparing the difference value to a threshold difference value; and
in response to a determination that the difference value meets or exceeds the threshold difference value, establishing the threshold ratio value based on the first ratio value and the second ratio value.

6. The method of claim 5, further comprising:
generating at least one histogram representation of values for each ratio of the at least one ratio, and
determining the ratio value associated with the predetermined percentile for each known target based on the histogram.

7. The method of claim 6, further comprising determining the threshold difference value, wherein determining the threshold difference value comprises:
comparing a first difference value associated with a histogram generated using a first ratio of the at least one ratio to a second difference value associated with a histogram generated using a second ratio of the at least one ratio; and
determining whether the first difference value or the second difference value is larger; and
in response to a determination that the first difference value is larger than the second difference value, selecting the first difference value as the threshold difference value, or
in response to a determination that the second difference value is larger than the first difference value, selecting the second difference value as the threshold difference value.

8. The method of claim 7, further comprising assigning a weighting factor to the ratio associated with the largest difference value.

9. The method of claim 5, wherein the threshold ratio value is based on an average of the first and second ratio values.

10. The method of claim 5, wherein the predetermined percentile is the 80^{th} percentile.

11. The method of claim 4, wherein generating the optical data includes determining the at least one ratio for the target in the surgical treatment area, and identifying the target comprises:
comparing a ratio value of the at least one ratio for the target in the surgical treatment area to the threshold ratio value; and
associating the target in the surgical treatment area with a known target of the at least two known targets based on the comparison.

12. The method of claim 11, further comprising:
determining whether the known target is a treatment target; and
in response to a determination that the known target is a treatment target, identifying the target as a treatment target, or
in response to a determination that the known target is not a treatment target, identifying the target as a non-treatment target.

13. The method of claim 12, wherein determining whether the known target is a treatment target is performed in between every N laser pulses emitted by a treatment laser.

14. The method of claim 12, wherein determining whether the known target is a treatment target is performed after modifying a laser operating parameter of a treatment laser.

15. The method of claim 4, wherein establishing the threshold ratio value further comprises defining a multidimensional decision space having n decision options based on at least two ratios of the at least one ratio, where n is the number of known targets.

16. The method of claim 14, further comprising defining multidimensional threshold separation lines between the n decision options in the multidimensional decision space for discrimination between each target of the known targets.

17. The method of claim 1, wherein the computing device is further configured to generate a control signal for controlling operation of a treatment laser based on the identification of the target.

18. The method of claim 17, wherein the control signal includes activation, de-activation or an operating parameter setting for the treatment laser.

19. The method of claim 1, wherein the computing device is further configured to generate an audio, visual, or tactile signal to an operator based on the identification of the target.

20. The method of claim 1, wherein the treatment target is a stone and the non-treatment target is tissue or a surgical component or a surgical treatment area medium.

21. The method of claim 1, wherein the computing device is configured to couple with three photodetectors and the three different selected wavelength bands are selected from a group consisting of: about 400-410 nm, about 440-480 nm, about 460-480 nm, about 510-530 nm, about 540-560 nm, about 550-570 nm, about 570-580 nm, about 580-600 nm, about 600-620 nm, about 690-710 nm, about 740-760 nm, about 790-810 nm, about 920-940 nm, about 970-990 nm, and about 1150-1350 nm.

22. The method of claim 1, wherein the computing device is further configured to identify the target as a treatment target or a non-treatment target based at least in part on a comparison against stored data from previously recorded reflected intensity values.

23. The method of claim 1, wherein the computing device is further configured to identify the target as a treatment target or a non-treatment target based at least in part on a machine learning model.

24. A surgical laser system comprising:
a surgical fiber configured to receive light reflected by a target in a surgical treatment area; and
a computing device configured to couple with at least two photodetectors, each photodetector configured to detect an intensity of reflected light from the target in a difference selected wavelength band, and configured to:
receive the reflected light intensity in at least two selected wavelength bands;
generate optical data corresponding to the reflected light intensity; and
identify the target as a treatment target or a non-treatment target based at least in part on the optical data and a predetermined calibration based on at least two known targets.

25. The surgical laser system of claim 24, wherein the computing device is further configured to perform the calibration.

26. The surgical laser system of claim 25, wherein at least one of generating the optical data and performing the calibration includes determining at least one ratio of a reflected light intensity of one selected wavelength band to a reflected light intensity of a different selected wavelength band.

27. The surgical laser system of claim 26, wherein performing the predetermined calibration further comprises:
obtaining multiple reflected light intensity values from each known target of the at least two known targets; and
establishing a threshold ratio value based at least in part on the multiple reflected light intensity values from each known target.

28. The surgical laser system of claim 27, further comprising:
determining a ratio value associated with a predetermined percentile for each known target based on the multiple reflected light intensity values from each known target;
determining a difference value between a first ratio value associated with the predetermined percentile for a first known target and a second ratio value associated with the predetermined percentile for a second known target;
comparing the difference value to a threshold difference value; and
in response to a determination that the difference value meets or exceeds the threshold difference value, establishing the threshold ratio value based on the first ratio value and the second ratio value.

29. The surgical laser system of claim 28, further comprising:
generating at least one histogram representation of values for each ratio of the at least one ratio, and
determining the ratio value associated with the predetermined percentile for each known target based on the histogram.

30. The surgical laser system of claim 29, wherein the computing device is further configured to determine the threshold difference value, and determining the threshold difference value comprises:
comparing a first difference value associated with a histogram generated using a first ratio of the at least one ratio to a second difference value associated with a histogram generated using a second ratio of the at least one ratio; and
determining whether the first difference value or the second difference value is larger; and
in response to a determination that the first difference value is larger than the second difference value, selecting the first difference value as the threshold difference value, or
in response to a determination that the second difference value is larger than the first difference value, selecting the second difference value as the threshold difference value.

31. The surgical laser system of claim 27, wherein generating the optical data includes determining the at least one ratio for the target in the surgical treatment area, and identifying the target comprises:
comparing a ratio value of the at least one ratio for the target in the surgical treatment area to the threshold ratio value; and
associating the target in the surgical treatment area with a known target of the at least two known targets based on the comparison.

32. The surgical laser system of claim 31, further comprising:
determining whether the known target is a treatment target; and
in response to a determination that the known target is a treatment target, identifying the target as a treatment target, or
in response to a determination that the known target is not a treatment target, identifying the target as a non-treatment target.

33. The surgical system of claim 24, wherein the light reflected from the target is broadband light and the different selected wavelength bands include wavelength bands selected from the list consisting of: about 400-410 nm, about 440-480 nm, about 460-480 nm, about 510-530 nm, about 540-560 nm, about 550-570 nm, about 570-580 nm, about 580-600 nm, about 600-620 nm, about 690-710 nm, about 740-760 nm, about 790-810 nm, about 920-940 nm, about 970-990 nm, and about 1150-1350 nm.

34. The surgical system of claim 24, further comprising a treatment laser, and the computing device is further configured to generate a control signal for controlling operation of the treatment laser based on the identification of the target.

35. The surgical system of claim 24, wherein the treatment target is a stone and the non-treatment target is tissue or a surgical component or a surgical treatment area medium.
